(19) United States
(12) Patent Application Publication
(10) Pub. No.: US 2023/0320642 A1
(43) Pub. Date: Oct. 12, 2023

(54) SYSTEMS AND METHODS FOR TECHNIQUES TO PROCESS, ANALYZE AND MODEL INTERACTIVE VERBAL DATA FOR MULTIPLE INDIVIDUALS

(71) Applicant: The Trustees of Columbia University in the City of New York, New York, NY (US)

(72) Inventor: Baihan Lin, New York, NY (US)

(21) Appl. No.: 18/130,947

(22) Filed: Apr. 5, 2023

Related U.S. Application Data
(60) Provisional application No. 63/409,373, filed on Sep. 23, 2022.
(60) Provisional application No. 63/402,534, filed on Aug. 31, 2022.
(60) Provisional application No. 63/389,131, filed on Jul. 14, 2022.
(60) Provisional application No. 63/351,991, filed on Jun. 14, 2022.
(60) Provisional application No. 63/351,579, filed on Jun. 13, 2022.
(60) Provisional application No. 63/329,615, filed on Apr. 11, 2022.
(60) Provisional application No. 63/328,787, filed on Apr. 8, 2022.

(51) Int. Cl. A61B 5/16; G16H 20/70; G10L 17/02; G10L 17/14; G10L 17/22; G10L 25/66; G10L 17/18; G10L 21/028; A61B 5/00

(52) U.S. Cl. CPC A61B 5/165; G16H 20/70; G10L 17/02; G10L 17/14; G10L 17/22; G10L 25/66; G10L 17/18; G10L 21/028; A61B 5/4803; A61B 5/7267

(57) ABSTRACT

Disclosed are methods, systems, and other implementations for processing, analyzing, and modelling psychotherapy data. The implementations include a method for analyzing psychotherapy data that includes obtaining transcript data representative of spoken dialog in one or more psychotherapy sessions conducted between a patient and a therapist, extracting speech segments from the transcript data related to one or more of the patient or the therapist, applying a trained machine learning topic model process to the extracted speech segments to determine weighted topic labels representative of semantic psychiatric content of the extracted speech segments, and processing the weighted topic labels to derive a psychiatric assessment for the patient.

### CROSS-REFERENCE TO RELATED APPLICATIONS

This application claims the benefit of, and priority to, U.S. Provisional Application Nos. 63/328,787, entitled “SYSTEMS AND METHODS FOR TOPIC MODELING FOR PSYCHOTHERAPY SESSIONS,” and filed April 8, 2022; 63/329,615, entitled “SYSTEMS AND METHODS FOR AUTOMATIC MONITORING AND DIAGNOSING OF MENTAL CONDITIONS USING PSYCHOTHERAPY DATA” and filed Apr. 11, 2022; 63/351,579, entitled “SYSTEMS AND METHODS FOR UNSUPERVISED INFERENCE OF CONVERSATIONAL PERSONALITY TYPES USING DIALOGUE DATA” and filed Jun. 13, 2022; 63/402,534 entitled “SYSTEMS AND METHODS FOR SUPPORTING PSYCHOTHERAPY WITH REAL-TIME RECOMMENDATIONS OF TREATMENT STRATEGIES” and filed Aug. 31, 2022; 63/389,131 entitled “SYSTEMS AND METHODS FOR SUPPORTING PSYCHOTHERAPY WITH REAL-TIME RECOMMENDATIONS OF TREATMENT STRATEGIES” and filed Jul. 14, 2022; 63/409,373 entitled “SYSTEMS AND METHODS FOR SPEAKER DIARIZATION” and filed Sep. 23, 2022; 63/351,991 entitled “SYSTEMS AND METHODS FOR KNOWLEDGE MANAGEMENT WITH RELATIONAL DATABASES” and filed Jun. 14, 2022, the contents of all of which are incorporated herein by reference in their entireties.

### BACKGROUND

Mental health remains an issue in all countries and cultures across the globe. According to the National Institute of Mental Health (NIMH), nearly one in five U.S. adults lives with a mental illness (52.9 million in 2020). One of the major causes of the mental illness is depression (which can lead to suicide, which is the second cause of death among young people).

Psychotherapy is a term given for treating mental health problems by talking with a mental health provider such as a psychiatrist or psychologist. Psychotherapy is based on the exchange between individuals and therapists, relying on self-report measures and humans to quantify sessions. While these standard methods are the building blocks of the field, they have shortcomings, including an individual's willingness to participate and the limitations and preconceptions of a therapist's notes. This leads to a highly qualitative understanding of a patient's state and progress that can change from therapist to therapist.

### SUMMARY

Disclosed are implementations (including hardware, software, and hybrid hardware/software implementations) directed to several machine-learning-based frameworks and techniques for processing and analyzing verbal input (usually in the form of transcripts or captured speech) from interactive sessions (such as patient-therapist psychotherapy sessions, group therapy, etc.) and providing behavior, therapeutic, or training related outputs that can assist various entities (be it seasoned or in-training therapists, or behavior analysis persons or systems) to store, model, analyze, and respond to the verbal input.

In some variations, a first method, for analyzing psychotherapy data, is provided that includes obtaining transcript data representative of spoken dialog in one or more psychotherapy sessions conducted between a patient and a therapist, extracting speech segments from the transcript data related to one or more of the patient or the therapist, applying a trained machine learning topic model process to the extracted speech segments to determine weighted topic labels representative of semantic psychiatric content of the extracted speech segments, and processing the weighted topic labels to derive a psychiatric assessment for the patient.

In some variations, a first system, for psychotherapy data analysis, is provided that includes a communication unit to obtain transcript data representative of spoken dialog in one or more psychotherapy sessions conducted between a patient and a therapist, and a processor-based controller coupled to the communication unit. The controller is configured to extract speech segments from the transcript data related to one or more of the patient or the therapist, apply a trained machine learning topic model process to the extracted speech segments to determine weighted topic labels representative of semantic psychiatric content of the extracted speech segments, and process the weighted topic labels to derive a psychiatric assessment for the patient.

In some embodiments, a first non-transitory computer readable media is provided that includes computer instructions executable on a processor-based device to obtain transcript data representative of spoken dialog in one or more psychotherapy sessions conducted between a patient and a therapist, extract speech segments from the transcript data related to one or more of the patient or the therapist, apply a trained machine learning topic model process to the extracted speech segments to determine weighted topic labels representative of semantic psychiatric content of the extracted speech segments, and process the weighted topic labels to derive a psychiatric assessment for the patient.

In some variations, a second method, for analyzing dialogue data, is provided that includes transforming one or more patient speech segments and one or more speech segments of at least another speaker, representative of spoken dialogue between a patient and the at least other speaker, into representations in a vector space to produce one or more vectored patient representations and one or more vectored speaker representations. The second method further includes determining one or more patient similarity scores between the one or more vectored patient representations and one or more vectored representations of a set of semantic elements in one or more inventories of cognitive properties, determining one or more speaker similarity scores between the one or more vectored speaker representations and one or more vectored representations of another set of semantic elements in the one or more inventories of cognitive properties, and determining based on the one or more patient similarity scores and the one or more speaker similarity scores a psychiatric assessment for the patient.

In some variations, a second system, for dialogue data analysis, is provided that includes one or more memory devices to store processor-executable instructions and dialogue data relating to one or more events involving a patient and at least another speaker, and a processor-based controller, coupled to the one or more memory devices. The processor-based controller is configured, when executing the processor-executable instructions, to transform one or more patient speech segments and one or more speech segments of the at least other speaker, representative of the dialogue data, into representations in a vector space to produce one or more vectored patient representations and one or more vectored speaker representations. The processor-based controller is further configured to determine one or more patient similarity scores between the one or more vectored patient representations and one or more vectored representations of a set of semantic elements in one or more inventories of cognitive properties, determine one or more speaker similarity scores between the one or more vectored speaker representations and one or more vectored representations of another set of semantic elements in the one or more inventories of cognitive properties, and determine based on the one or more patient similarity scores and the one or more speaker similarity scores a psychiatric assessment for the patient.

In some embodiments, a non-transitory computer readable media is provided that includes computer instructions executable on a processor-based device to transform one or more patient speech segments and one or more speech segments of at least another speaker, representative of spoken dialogue between a patient and the at least other speaker, into representations in a vector space to produce one or more vectored patient representations and one or more vectored speaker representations. The computer instructions further cause the processor-based device to determine one or more patient similarity scores between the one or more vectored patient representations and one or more vectored representations of a set of semantic elements in one or more inventories of cognitive properties, determine one or more speaker similarity scores between the one or more vectored speaker representations and one or more vectored representations of another set of semantic elements in the one or more inventories of cognitive properties, and determine based on the one or more patient similarity scores and the one or more speaker similarity scores a psychiatric assessment for the patient.

In some variations, a third method, for processing psychotherapy session data, is provided that includes obtaining a current speech segment, representative of spoken dialogue between a patient and a therapist during a dialogue session comprising multiple speech segments, and transforming the current speech segment into a representation in a vector space to produce one or more vectored patient representations and one or more vectored therapist representations. The third method further includes determining one or more patient similarity scores between the one or more vectored patient representations and one or more vectored representations of a set of semantic elements in one or more inventories of cognitive properties, determining one or more therapist similarity scores between the one or more vectored therapist representations and one or more vectored representations of another set of semantic elements in the one or more inventories of cognitive properties, and determining based on the one or more patient similarity scores and/or the one or more therapist similarity scores therapist advice output to dynamically manage the dialogue session in real-time by identifying, in response to the current speech segment, therapy-relevant actionable items.

In some variations, a third system, for dynamic recommendation, is provided that includes a receiver module to obtain audio data for a patient-therapist dialogue session, and convert least part of the audio data into a current speech segment, and a processor-based controller, coupled to the one or more memory devices. The controller is configured to transform the current speech segment into a representation in a vector space to produce one or more vectored patient representations and one or more vectored therapist representations, determine one or more patient similarity scores between the one or more vectored patient representations and one or more vectored representations of a set of semantic elements in one or more inventories of cognitive properties, determine one or more therapist similarity scores between the one or more vectored therapist representations and one or more vectored representations of another set of semantic elements in the one or more inventories of cognitive properties, and determine based on the one or more patient similarity scores and/or the one or more therapist similarity scores therapist advice output to dynamically manage the dialogue session in real-time by identifying, in response to the speech segment, therapy-relevant actionable items.

In some embodiments, a third non-transitory computer readable media is provided that includes computer instructions executable on a processor-based device to transform the current speech segment into a representation in a vector space to produce one or more vectored patient representations and one or more vectored therapist representations, determine one or more patient similarity scores between the one or more vectored patient representations and one or more vectored representations of a set of semantic elements in one or more inventories of cognitive properties, determine one or more therapist similarity scores between the one or more vectored therapist representations and one or more vectored representations of another set of semantic elements in the one or more inventories of cognitive properties, and determine based on the one or more patient similarity scores and/or the one or more therapist similarity scores therapist advice output to dynamically manage the dialogue session in real-time by identifying, in response to the speech segment, therapy-relevant actionable items.

In some variations, a fourth method, for multi-speaker diarization, is provided that includes obtaining a speech segment, extracting one or more speech features from the speech segment, processing the one or more extracted speech features with a configurable machine learning diarization engine adapted to identify a speaker associated with the speech segment, and adjusting weights of the configurable machine learning diarization engine according to one or more reinforcement learning approaches in response to receipt of feedback indicative of accuracy of the speaker identified by the diarization engine to a true speaker identity for the speech segment.

In some variations, a fourth system, for diarization, is provided that includes a receiver module to obtain a speech segment, and a processor-based controller, coupled to one or more memory devices. The controller is configured to extract one or more speech features from the speech segment, process the one or more extracted speech features with a configurable machine learning diarization engine adapted to identify a speaker associated with the speech segment, and adjust weights of the configurable machine learning diarization engine according to one or more reinforcement learning approaches in response to receipt of feedback indicative of accuracy of the speaker identified by the diarization engine to a true speaker identity for the speech segment.

In some embodiments, a fourth non-transitory computer readable media is provided that includes computer instructions executable on a processor-based device to extract one or more speech features from the speech segment, process the one or more extracted speech features with a configurable machine learning diarization engine adapted to identify a speaker associated with the speech segment, and adjust weights of the configurable machine learning diarization engine according to one or more reinforcement learning approaches in response to receipt of feedback indicative of accuracy of the speaker identified by the diarization engine to a true speaker identity for the speech segment.

In some variations, a fifth method, for knowledge management processing, is provided that includes obtaining a particular document, determining at a first time instance metadata information elements associated with the particular document, and including in a particular record of a relational database associated with the particular document at least some of the metadata information elements determined at the first time instance in one or more of a plurality of fields of the particular record. The plurality of fields includes at least: a) a document-specific concepts field to maintain concepts specific to the particular document, and b) common concepts field to maintain common concepts shared by a plurality of documents associated with a plurality of records in the relational database. The procedure further comprises including in the particular record of the relational database, at one or more subsequent time instances, one or more document-specific user notes for storage in a document-specific notes field, and one or more general document user notes, determined by a machine learning engine analyzing other records in the relational database, for storage in a common notes field of multiple records of the relational database sharing the general user notes.

In some variations, a fifth system, for knowledge management, is provided that includes a user interface to provide input and present output relating to one or more documents, one or more memory devices to maintain a relational database storing information relating to the one or more documents, and a processor-based controller, in communication with the user interface and the one or more memory devices. The controller is configured, for a particular document, to determine at a first time instance metadata information elements associated with the particular document, and include in a particular record of the relational database associated with the particular document at least some of the metadata information elements determined at the first time instance in one or more of a plurality of fields of the particular record. The plurality of fields includes at least, for example, a) a document-specific concepts field to maintain concepts specific to the particular document, and b) common concepts field to maintain common concepts shared by a plurality of documents associated with a plurality of records in the relational database. The controller is further configured to include in the particular record of the relational database, at one or more subsequent time instances, one or more document-specific user notes for storage in a document-specific notes field, and one or more general documents user notes, determined by a machine learning engine analyzing other records in the relational database, for storage in a common notes field of multiple records of the relational database sharing the general documents user notes.

In some embodiments, a fifth non-transitory computer readable media is provided that includes computer instructions executable on a processor-based device to obtain a particular document, determining at a first time instance metadata information elements associated with the particular document, and include in a particular record of a relational database associated with the particular document at least some of the metadata information elements determined at the first time instance in one or more of a plurality of fields of the particular record. The plurality of fields includes at least: a) a document-specific concepts field to maintain concepts specific to the particular document, and b) common concepts field to maintain common concepts shared by a plurality of documents associated with a plurality of records in the relational database. The computer instructions include some additional instructions to include in the particular record of the relational database, at one or more subsequent time instances, one or more document-specific user notes for storage in a document-specific notes field, and one or more general document user notes, determined by a machine learning engine analyzing other records in the relational database, for storage in a common notes field of multiple records of the relational database sharing the general user notes.

In some variations, a sixth method, for visual representation of psychotherapy data, is provided that includes obtaining transcript data representative of spoken dialog in one or more psychotherapy sessions conducted between a patient and a therapist, extracting speech segments from the transcript data related to one or more of the patient or the therapist, applying a trained machine learning topic model process to the extracted speech segments to determine a temporal series of topic labels representative of semantic psychotherapy content of the extracted speech segments, determining a temporal visual representation of one or more of, for example, the topic labels of the temporal series and/or the transcript data, and rendering the temporal visual representation on an output user interface.

In some variations, a sixth system for visual representation of psychotherapy data is provided. The system includes a user interface to provide input and present output relating to the psychotherapy data, one or more memory devices to maintain time-dependent data associated with the psychotherapy data, and a processor-based controller in communication with the user interface and the one or more memory devices. The processor-based controller is configured to obtain transcript data representative of spoken dialog in one or more psychotherapy sessions conducted between a patient and a therapist, extract speech segments from the transcript data related to one or more of the patient or the therapist, apply a trained machine learning topic model process to the extracted speech segments to determine a temporal series of topic labels representative of semantic psychotherapy content of the extracted speech segments, determine a temporal visual representation of one or more of, for example, the topic labels of the temporal series and/or the transcript data, and render the temporal visual representation on an output device of the user interface.

In some embodiments, a sixth non-transitory computer readable media is provided that includes computer instructions executable on a processor-based device to obtain transcript data representative of spoken dialog in one or more psychotherapy sessions conducted between a patient and a therapist, extract speech segments from the transcript data related to one or more of the patient or the therapist, apply a trained machine learning topic model process to the extracted speech segments to determine a temporal series of topic labels representative of semantic psychotherapy content of the extracted speech segments, determine a temporal visual representation of one or more of, for example, the topic labels of the temporal series and/or the transcript data, and render the temporal visual representation on an output user interface.

Embodiments and variations of any of first, second, third, fourth, fifth, and sixth methods, systems, and computer readable media may include at least some of the features described in the present disclosure, including at least some of the features described above in relation to the methods, the systems, and the computer-readable media. Furthermore, any of the above variations and embodiments of the methods, systems, and/or computer-readable media, may be combined with any of the features of any other of the variations of the methods, systems, and computer-readable media described herein, and may also be combined with any other of the features described herein.

Other features and advantages of the invention are apparent from the following description, and from the claims.

### BRIEF DESCRIPTION OF THE DRAWINGS

These and other aspects will now be described in detail with reference to the following drawings.

FIG. 1 is a schematic diagram of an example psychotherapy analytic framework.

FIG. 2 is a flowchart of a procedure for analyzing psychotherapy data.

FIG. 3 includes tables summarizing quantitative evaluations of neural topic models.

FIG. 4 is a graph showing the average 3D temporal trajectories of the patients and therapists in a principal topic spaces.

FIG. 5 is a heatmap of averaged topic scores.

FIG. 6 is a schematic diagram of an example analytic framework to infer cognitive anchors according to one or more psychological inventories using conversational data.

FIG. 7 is a flowchart for a procedure for analyzing dialogue data.

FIG. 8 includes a table with the empirical evaluation results of the Kaggle MBTI post classification task.

FIG. 9 includes graphs showing the session-wise working alliance scores in the three scales and their relationship to the personality consistency between the therapist and the patient.

FIG. 10 is a schematic diagram of a recommendation system.

FIG. 11 is a schematic diagram of an example reinforcement learning framework for the recommendation system of FIG. 10.

FIG. 12 is a flowchart of an example procedure for processing psychotherapy session data and making treatment strategy recommendations.

FIG. 13 includes state screenshots of SupervisorBot web app system with inventory inputs, diarization training, state annotation, and strategy recommendations panels.

FIG. 14 is a diagram of the reinforcement learning diarization system.

FIG. 15 includes diagrams illustrating operations of four different classes of reinforcement learning processes that may be used in conjunction with the diarization system of FIG. 14

FIG. 16 is a flowchart of a procedure for multi-speaker diarization.

FIG. 17 is a schematic diagram of a knowledge management system with relational databases and insight annotation powered by natural language processing (NLP).

FIG. 18 is a flowchart of a procedure for knowledge management processing.

FIG. 19 s a screenshot of an example dashboard of a TherapyView platform to provide visualization based on psychotherapy data.

FIG. 20 is a flowchart of an example procedure for visual representation of psychotherapy data.

FIG. 21 is a diagram of an example architecture of a working alliance transformer (WAT) for psychiatric condition classification using a psychological state encoder.

FIG. 22 is a schematic diagram of a reinforcement learning psychotherapy AI companion system with disorder-specific multi-objective policies (DISMOP).

FIG. 23 is a schematic diagram of an analytic framework of the AI companion implementation.

FIG. 24 includes a graph table presenting the standardized average policy trajectories with respect to the action embeddings projected onto a 2D principal component analysis space.

FIG. 25 is a graph table of 1-step transition matrices of trained disorder-specific policies.

Like reference symbols in the various drawings indicate like elements.

### DESCRIPTION

Below are detailed descriptions of several proposed frameworks for analyzing interactive verbal input (e.g., transcripts) to derive output (e.g., for behavioral analysis and proposed therapy solutions).

### Framework 1: Topic Modeling for Psychotherapy Sessions

In a first set of example embodiments, an analytical system that performs topic modeling on transcripts of psychotherapy sessions is described. Snippets from the transcripts are extracted and are fitted into topic models. The resultant weighted list of topic words is then processed by downstream processes to, for example, analyze whether the therapy is going in the right direction, whether the patient is moving into a bad mental state, or whether the therapist should adjust his or her treatment strategies. In machine learning and natural language processing, a topic model is a type of statistical model for discovering the abstract “topics” that occur in a collection of documents. Topic modeling is a text-mining tool for discovery of hidden semantic structures in a text body. The proposed framework of the first set of example embodiments described herein (referred to as topic modeling for psychotherapy sessions) incorporates temporal modeling to put this additional interpretability to action by parsing out topic similarities as a time series in a turn-level resolution. Such a topic modeling framework can offer interpretable insights for the therapist to optimally decide his or her strategy and improve the psychotherapy effectiveness.

Framework 1 implements a topic modeling process for mental health evaluation to analyze text from therapy sessions and generate information about the process and outcomes. The technology can analyze patient and provider (therapist) dialogues together and separately. The framework facilitates the learning the topical propensities of different psychiatric conditions from the psychotherapy session transcripts (e.g., parsed from speech recordings).

The outputs of Framework 1 are designed to be easily interpretable, making the system easy to transition into user-friendly interfaces. Potential applications of this technology include a method to quantitatively evaluate therapy and increase the effectiveness of digital mental health care, including AI-based mental health chat applications.

A topic model is a type of statistical graphical model that help uncover the abstract “topics” that appear in a collection of documents. The topic modeling technique is used in text-mining pipeline to unravel the hidden semantic structures of a text body. Several neural topic models may be used in conjunction with Framework 1, including the Neural Variational Document Model (NVDM), which is an unsupervised text modeling approach based on variational auto-encoder. Among NVDM variants, the Gaussian softmax construction (GSM) has been shown to achieve the lowest perplexity in most cases (this modelling is referred to NVDM-GSM). Another topics model that may be used is the Wasserstein-based Topic Model (WTM). Unlike traditional variational autoencoder based methods, WTM uses the Wasserstein autoencoders (WAE) to directly enforce Dirichlet prior on the latent document-topic vectors. Traditionally, it applies a suitable kernel in minimizing the Maximum Mean Discrepancy (MMD) to perform distribution matching (this variant can be referred to as WTM-MMD). Similarly, in some embodiments, the MMD priors can be replaced with a Gaussian Mixture prior and have a Gaussian Softmax applied on top of it (this is referred to a WTM-GMM). In order to tackle the issue with large and heavy-tailed vocabularies, the Embedded Topic Model (ETM) models each word with a matched categorical probability distribution given the inner product between a word embedding and a vector embedding of its assigned topic. To avoid imposing improper priors, Bidirectional Adversarial Training Model (BATM) applies the bidirectional adversarial training into neural topic modeling by constructing a two-way projection between the document-word distribution and the document topic distribution.

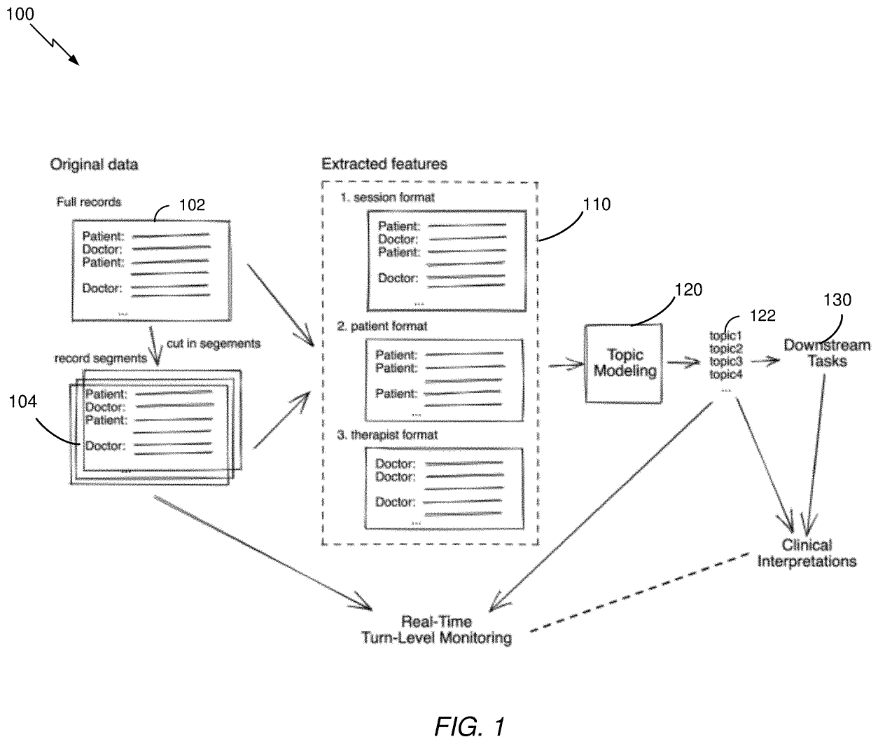

With reference to FIG. 1, a schematic diagram of an example proposed psychotherapy analytic framework 100 is shown. During a psychotherapy session, the dialogue between the patient and therapist are transcribed into pairs of turns Under the proposed approach, the full records of a patient, or a cohort of patients having the same or similar condition, are obtained (depicted as original data 102 in FIG. 1). The transcribed dialog can be used as-is, can be truncated into shorter segments (depicted in FIG. 1 as cut in segments 104) based on timestamps or topic turns. For example, when a transcript is provided as a paired dialog (between a patient and a therapist), features in the transcript can be extracted (at box 110) in one of several ways. In a first example extraction strategy, the full set of dialogue pairs is provided as input to a processing engine (i.e., what is processed are dialog pairs, whether broken down to one turn segments, or to longer multi-turn segments). A second example extraction strategy is one in which only the patient's transcribed content is used as the input data. In a third strategy, only the therapist's transcribed content is used as input data (either one turn at a time, or multi-turns at a time). Of course, other sequential data extraction strategies may be used, e.g., grouping patient's content from multi-turns together, grouping the therapist's content from multi-turns together, and processing the patient's and therapist's content groupings separately. The different feature extraction formats (strategies) all have their pros and cons. The dialogue format contains all the information obtained from a psychotherapy session, but the intent within the sentences comes from two individuals, so the data might result in confusion or ambiguity. The patient format contains the full narrative of the patients, which is usually more coherent, but is only part of the story. The therapist format, which can provide accurate semantic labels of what the patient feels, can be informative, but may also be sometimes too simplistic.

Once the features are extracted (under a selected extraction model), the extracted features are analyzed by topic modeling unit 120 using, for example, a trained machine learning topic model engines. The end result of the topic modeling is a list of weighted topic words 122, that can indicate or represent what a portion(s) of semantic content extracted from the transcript relates to. This knowledge can be very insightful and provide valuable interpretable information, and can thus be an important tool in psychotherapy applications.

In one example embodiment, a temporal topic modeling (or TMM) that scores the similarity between extracted semantic segments and a library of general topic concepts is implemented on the topic modeling unit 120 (to compute relevance of a snippet of dialog or monolog to the various topic concepts). Example operations/functions comprising the temporal topic modeling (TTM) process include the following.

Temporal Topic Modeling (TTM)

| | |
|---|---|
| 1: | Learned topics T as references |
| 2: | for i = 1, 2, ..., N do |
| 3: | Automatically transcribe dialogue turn pairs (S^{p}ᵢ, S^{t}ᵢ) |
| 4: | for Tⱼ ∈ topics T do |
| 5: | Topic score W^{pi}ⱼ = similarity(Emb(Tⱼ),Emb(S^{p}ᵢ )) |
| 6: | Topic score W^{ti}ⱼ = similarity(Emb(Tⱼ),Emb(S^{t}ᵢ)) |
| 7: | end for |
| 8: | end for |

Thus, given a set of learned topics, a patient-therapist transcript can be analyzed, through a machine learning engine that transforms semantic content of some pre-determined size into a vector quantity in a trained vector space (also referred to as an embedding space), to get turn-resolution topic scores. For example, suppose that for the above operational pipeline of TTM analysis there are 10 learned topics/concepts (of course, there may be any other number of topics). In some embodiments, the machine learning engine may be implemented to transform transcript data into a vector/embedding space representation of semantic content, in which each topic/concept may map into a vector within the vector/embedding space that is representative of that particular topic or concept. In some implementation, other trainable learning models or configurations may be used to represent the various topics with respect to which analysis of the semantic content is performed). Assume, for the present example, that the machine learning model is one based on a vector/embedding space transformation. In that case, a topic score will be generated that is a vector of, for example, 10 dimensions, with each dimension corresponding to some notion of likelihood of the current snippet (semantic content turn) being related to that topic.

To characterize the directional property of each turn (snippet) with a certain topic, in some embodiments the cosine similarity of an embedded topic vector and the embedded turn vector are derived, instead of directly inferring the probability as traditional topic assignment problem (which might be more suitable if the goal were to find the assignment of the most likely topic). It is to be noted that an advantage of using a vector/embedded topic model approach is that such an approach can model each word with a categorical distribution whose natural parameter is the inner product between a word embedding and an embedding of its assigned topic. In some examples, the same word embedding transform (e.g., a Word2Vec implementation, a Bidirectional Encoder Representations from Transformers (BERT), or some other vector-space transformation implementation based on another language transform model) may be used to generate embedding for the topics and turns.

In some examples, other types of topic modeling implementations may be used, including some based on natural language processing. For example, Latent Dirichlet Allocation (LDA) is a popular method to extract relationships between multiple documents in a corpus. Other topic modeling methods include Non Negative Matrix Factorization (NMF), Latent Semantic Analysis (LSA), Pachinko Allocation Model (PAM), etc. Neural network-based topic modeling methodologies can be highly effective, and may include, but not limited to, Neural Variational Document Model (NVDM), Wasserstein Latent Dirichlet Allocation (W-LDA), Embedded Topic Models (ETM), and/or Bidirectional Adversarial Topic model (BATM).

There are a few downstream tasks (represented as tasks 130) and user scenarios that can be used in conjunction with the proposed analytical frameworks. For example, the extracted weighted topics can be used to inform whether the therapy is going the right direction, whether the patient is going into certain bad mental state, or whether the therapist should adjust his or her treatment strategies. This downstream analysis stage can be implemented as an intelligent AI assistant to the therapist of such things. In some embodiments, the downstream analysis stage can generate an alert for certain identified topic labels that indicate an emergency, such as topics indicating suicidal tendencies. Thus, if topics generated by the topic modeling engine are determined (through downstream analysis by, for example, a learning machine engine) to constitute an emergency, the therapist can be alerted through a notification sent to a computing device (e.g., a mobile computing device) associated with the therapist.

Thus, in some embodiments, a psychotherapy data analysis system is provided that includes a communication unit to obtain transcript data representative of spoken dialog in one or more psychotherapy sessions conducted between a patient and a therapist, and a processor-based controller coupled to the communication unit. The controller is configured to extract speech segments from the transcript data related to one or more of the patient or the therapist, apply a trained machine learning topic model process to the extracted speech segments to determine weighted topic labels representative of semantic psychiatric content of the extracted speech segments, and process the weighted topic labels to derive a psychiatric assessment for the patient. In some embodiments, a non-transitory computer readable media is provided that includes computer instructions executable on a processor-based device to obtain transcript data representative of spoken dialog in one or more psychotherapy sessions conducted between a patient and a therapist, extract speech segments from the transcript data related to one or more of the patient or the therapist, apply a trained machine learning topic model process to the extracted speech segments to determine weighted topic labels representative of semantic psychiatric content of the extracted speech segments, and process the weighted topic labels to derive a psychiatric assessment for the patient.

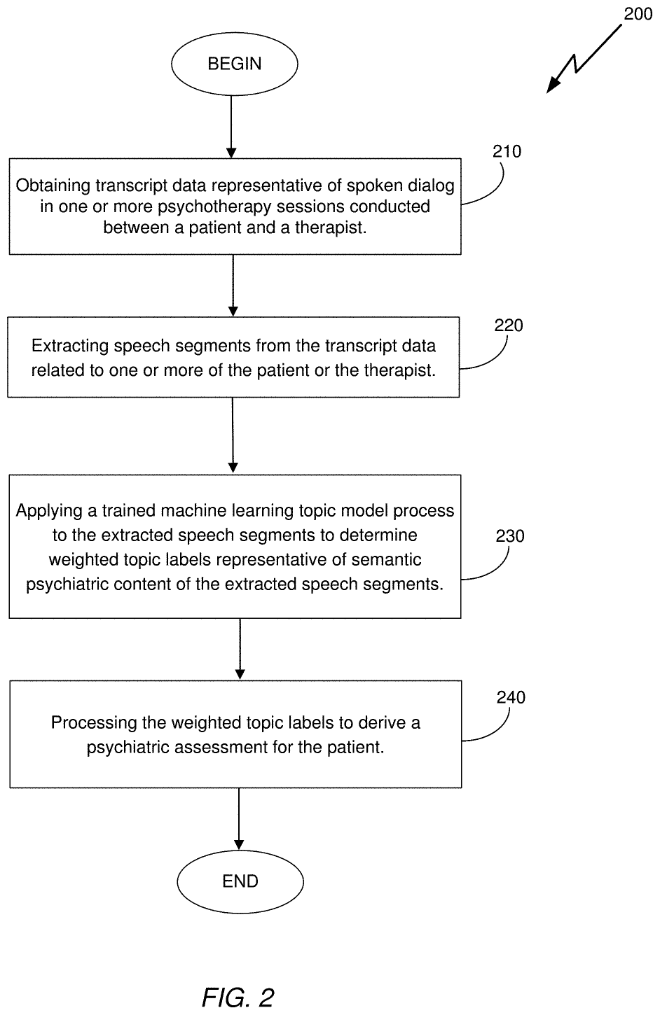

With reference next to FIG. 2, a flowchart of an example procedure 200 for analyzing psychotherapy data is provided. The procedure includes obtaining 210 transcript data representative of spoken dialog in one or more psychotherapy sessions conducted between a patient and a therapist, extracting 220 speech segments from the transcript data related to one or more of the patient or the therapist, applying 230 a trained machine learning topic model process to the extracted speech segments to determine weighted topic labels representative of semantic psychiatric content of the extracted speech segments, and processing 240 the weighted topic labels to derive a psychiatric assessment for the patient.

In some examples, the derived psychiatric assessment for the patient may include one or more of, for example, mental state of the patient, therapy adjustment recommendation, and/or trajectory of therapy for the patient. Processing the weighted topic labels may include applying a machine learning model to the weighted topic labels. In some examples, applying the topic model process to the extracted speech segments may include transforming one or more of the extracted speech segments into representations in a vector space to produce one or more vectored topic label representations, and determining one or more topic similarity scores between the one or more vectored topic label representations and one or more vectored representations of learned psychotherapy topic models. Extracting the speech segments from the transcript data related to one or more of the patient or the therapist may include extracting sequential temporal segments from the transcript data according to one or more extraction models that include, for example, pairing of dialog exchanges between the patient and the therapist, isolated patient-only speech segments, and/or isolated therapist-only speech segments.

In some examples, applying the topic model process to the extracted speech segments may include applying one or more of: a Latent Dirichlet Allocation (LDA) process, a Non Negative Matrix Factorization (NMF) process, a Latent Semantic Analysis (LSA) process, a Pachinko Allocation Model (PAM) process Neural Variational Document Model (NVDM) process, Wasserstein Latent Dirichlet Allocation (W-LDA) process, Embedded Topic Models (ETM) process, and/or a Bidirectional Adversarial Topic model (BATM) process.

Implementations of proposed Framework 1 were tested and evaluated to study their efficacy and performance. Five state-of-the-art neural topic modeling approaches were tested, and their learned topics performance was analyzed. Transcript sessions were separated into three categories based on the psychiatric conditions of the patients (anxiety, depression, and schizophrenia), and the topic models were trained over each of them for over 100 epochs at a batch size of 16. As in the standard preprocessing of topic modeling training, the lower bound of count was set to be 3 for words to keep in topic training, and the ratio of upper bound of count for words to keep in topic training was set to be 0.3.

Topic models are usually evaluated with the likelihood of held-out documents and topic coherence. However, it was shown that a higher likelihood of held-out documents does not necessarily correlate to the human judgment of topic coherence. Therefore, a series of more validated measurements of topic coherence and diversity was adopted. In the first evaluation, four topic embedding coherence metrics (cᵥ, c_{w2v}, c_{uci}, cₙₚₘᵢ) were computed to evaluate the topics generated by various models. The higher these measurements, the better. In all experiments each topic is represented by the top ten (10) words according to the topic-word probabilities, and the four metrics are calculated using Gensim library. Other than these four topic embedding coherence evaluation provided by Gensim, two other useful metrics were included. A first metric was computed to represent an asymmetrical confirmation measure between top word pairs (smoothed conditional probability). In addition, the topic diversity was computed by taking the ratio between the size of vocabulary in the topic words and the total number of words in the topics. Similarly, the higher these two measures are, the better the topic models.

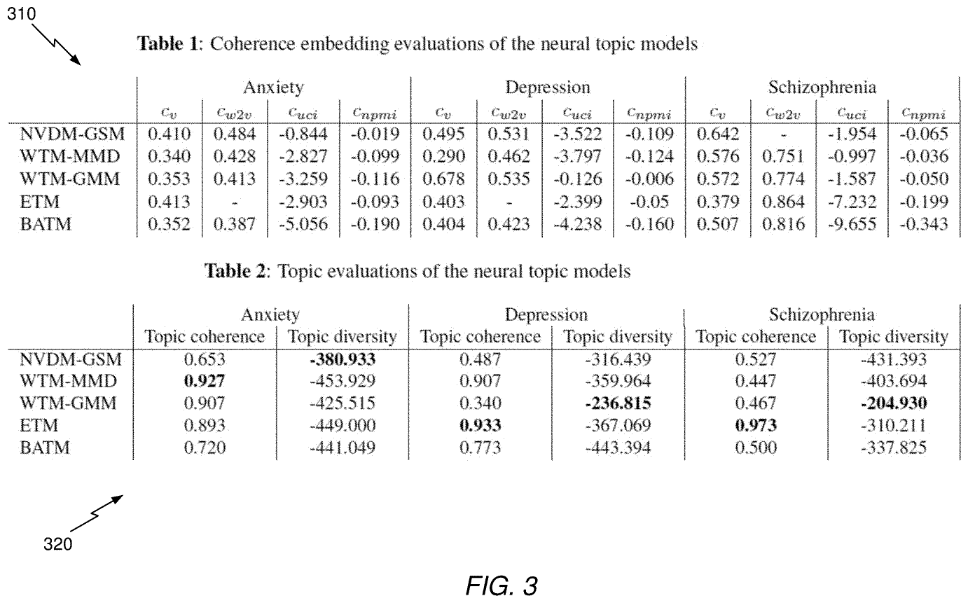

FIG. 3 includes two tables, 310 (Table 1) and 320 (Table 2) summarizing quantitative evaluations of neural topic models. It is first observed that the different measures of the coherence give different rankings of the topic models, but there are a few models that perform relatively well across the metrics. WTM and ETM both yield relatively high topic coherence and diversity.

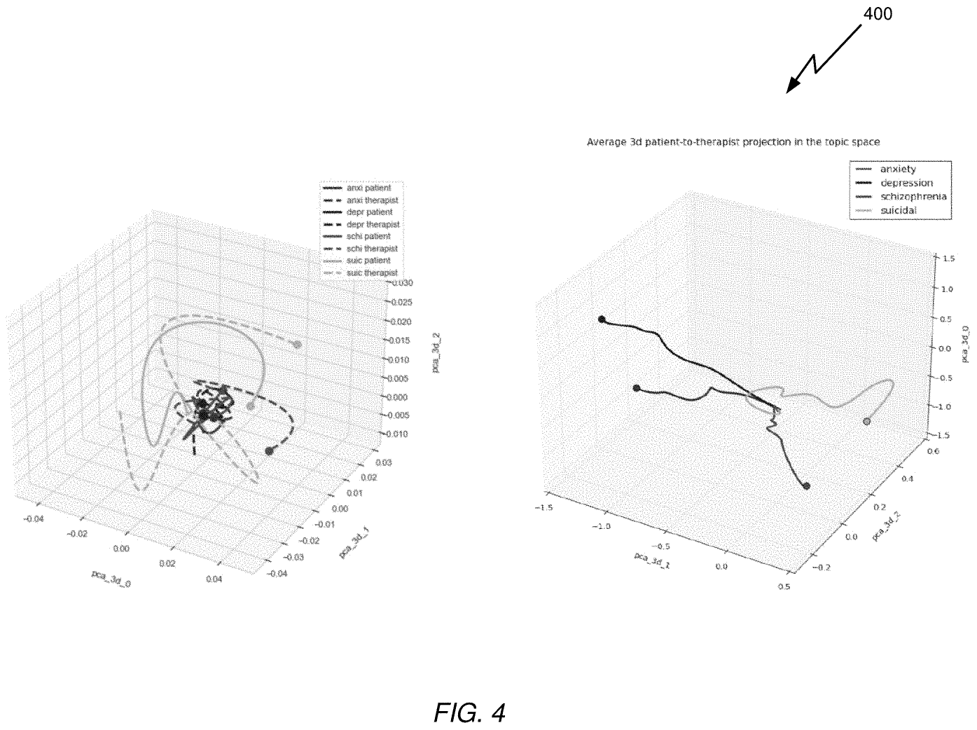

To ensure that the topics can be mapped from one clinical condition to another condition, a universal topic model was computed on the text corpus of the entire Alex Street psychotherapy database. Given the learned topics from this universal topic models, a 10-dimensional topic score was computed for each turn corresponding to the 10 topics. The higher the score is, the more positively correlated this turn is with this topic. Given this time-series matrix, the dynamics of these dialogues could be probed within the topic space. More distinctive features for downstream tasks can be provided by performing a principal component analysis on the topic space. FIG. 4 is a graph 400 showing the average 3D temporal trajectories of the patients and therapists, as well as the patient-to-therapist projection (i.e., the vector difference in the patient-therapist pair) in the principal topic spaces (in the graph, dots are the trajectory end points). It is observed that the suicidal sessions cover a wider variety of topics (by having more spread-out trajectories), and have a more curved patient-therapist topic difference with multiple twists along the full session, while the other three clinical conditions have more consistent directions of such differences. This might suggest that a strategy by the therapist to divert from the sensitive topics. In schizophrenia sessions, the therapist appears to cover a bigger topical arc than the patient, suggesting a therapeutic strategy of visiting multiple topics to distract the patient from sensitive ones. The topical trajectories of the anxiety and depression sessions, comparatively, are more converged. This is a first step of identifying the prototypical therapeutic strategies in different psychiatric conditions and a potential turn-level resolution temporal analysis of topic modeling.

To provide interpretable insights, it is important to parse out the concepts behind these learned topics. To better understand what these topics are, the highest scoring turns in the transcripts that correspond to each topics were parsed out. First, the individual topic models trained on text corpus of each psychiatric condition separately are considered. For instance, here are the interpretations from the top scoring turns in the anxiety sessions: topic 0 is chit-chat and interjections; topic 1 is low-energy exercises; topic 2 is fear; topic 3 is medication planning; topic 4 is the past, control and worry; topic 5 is other people and some objects; topic 6 is just wellbeing; topic 7 is music, headache, and emotion; topic 8 is stress; and topic 9 is fear and responsibilities. For depression, topic 0 is time; topic 1 is husband and anger; topic 2 is time and distance; topic 3 is energy and stress levels; topic 4 is self-esteem; topic 5 is money and time; topic 6 is age and time; topic 7 is mood and time; topic 8 is people and objects; topic 9 is holidays and chit-chats. For schizophrenia, topic 0 is family; topic 1 is extreme terms; topic 2 is energy level and positives; topic 3 is people and family; topic 4 is operational stuffs; topic 5 is calm things; topics 6 and 9 are critical topics. For the universal topic models, the results are much more coherent. For instance, topic 0 is about figuring out, self-discovery and reminiscence. Topic 1 is about play. Topic 2 is about anger, scare and sadness. Topic 3 is about counts. Topic 4 is about tiredness and decision. Topic 5 is about sickness, self-injuries, and coping mechanisms. Topic 6 is about explicit ways to deal with stress, such as keeping busy and reaching out for help. Topic 7 is about numbers. Topic 8 is about continuation and keep doing. Topic 9 is mostly chitchat, interjections, and transcribed prosody.

It is observed that among all the clinical conditions compared, the learned topics obtain a relatively poor mapping in the dialogue of suicidal cases. This might be due to the small sample size available in suicidal sessions, or the frequent hand annotations of behaviors (e.g., “patient crying for a few minutes” or “patient leaves the room”) with time stamps, which does not conform to the annotation style of other sessions.

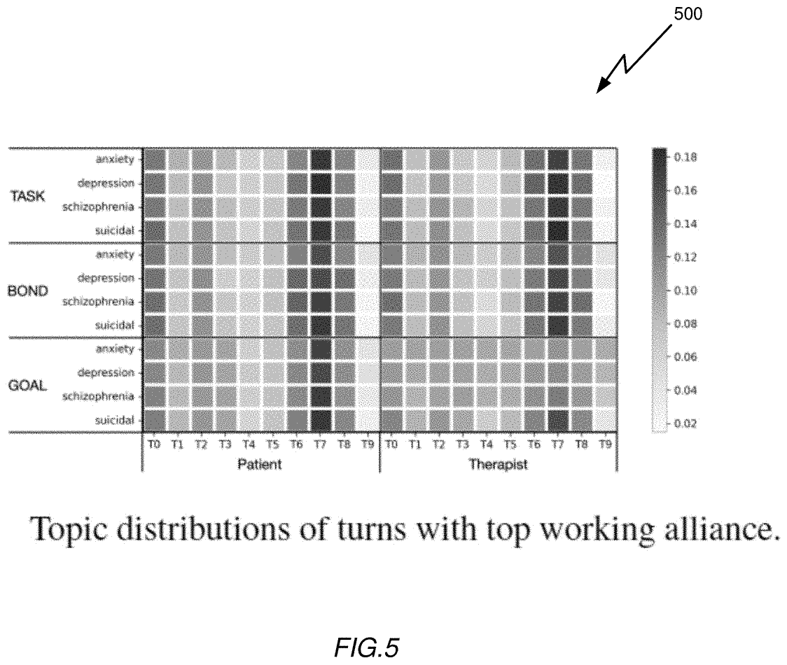

Although the approaches discussed herein can annotate the topics in each dialogue turns of the psychotherapy sessions, it is not clear how informative they might be from the therapeutic point of view. A computational technique to directly infer the therapeutic working alliance of a dialogue turn, which can be predictive of how effective the current therapy treatment is to the given patient at the given state, is proposed. Combining this method with the topic modeling framework allows highlighting disorder-specific topics and dialogue segments that are potentially indicative of the therapeutic breakthroughs. For each disorder, the turns to the top 100 working alliance scores are filtered separately in three scales (task, bond, and goal). FIG. 5 includes a heatmap 500 of the averaged topic scores. It is first observed that there is no clear distinction among the working alliance scales, but a relatively uniform coverage of the topics can be noticed when the patient and therapist are well aligned in the goal scale in all clinical conditions except for suicidal cases. Inspecting the top 10 turns with the highest topic scores, it is observed that within the turns with high working alliance goal scale, suicidal patients tend to discuss sensitive terms like “alive”, “stop” and “sexual.”

Thus, for the implementations of Framework 1 In this work, a first goal was to compare different neural topic modeling methods in learning the topical propensities of different psychiatric conditions. It was observed that different measures of the coherence give different rankings of the topic models, but there are a few topic models that perform relatively well across metrics. For instance, Wasserstein Topic Models and Embedded Topic Models both yield relatively high topic coherence and diversity. Another goal was to parse topics in different segments of the session, which allows incorporation of temporal modeling and additional interpretability. For instance, it was observed that the session trajectories of the patient and therapist are more separable from one another in anxiety and depression sessions, but more entangled in the schizophrenia sessions. This is the first step of a potential turn-level resolution temporal analysis of topic modeling.

The implementations of Framework 1 may further include predicting topic scores as states, training text or speech-based chatbots as reinforcement learning agents. Framework 1 may also be configured to construct a complete AI knowledge management system of mental health utilizing different NLP annotations in real time,

### Framework 2: Inferring Patient-Related Cognitive Characteristics Based on Conversational Data

The second proposed framework described herein (“Framework 2”) is an analytical framework of directly inferring patient-related cognitive characteristics, including personality traits (e.g., according to the Myers-Briggs scale) and therapeutic patient-therapist affinity (e.g., working alliance), based on conversational data (e.g., transcriptions of psychotherapy sessions) processed with machine learning systems that use, for example, deep embeddings models such as the Doc2Vec and SentenceBERT models. In various examples, the proposed framework extracts features from transcribed events (therapy sessions) using an encoder which may contain a word embedding layer to encode verbal content as numerical inputs, which can then be fed to, for example, a Bidirectional Encoder Representation from Transformers (BERT) model.

The Myers-Briggs type indicator (MBTI) has gained increasing popularity as an introspective self-report questionnaire to suggest the personality difference and psychological preferences in how people perceive the world around them and make decisions. The MBTI inventory is a set of questions that measures the personality traits in eight different scales. In various embodiments of the proposed approach, MBTI is used as a surrogate for the underlying individual personality because of the availability of existing datasets at a scale that contain both the tested personality labels and corresponding behavioral trajectories (like a post on a social media platform). Using machine learning models, the proposed approach aims to capture interpretable features of these behavioral trajectories to group individuals into different personality types, such that psychologists can gain more nuanced insights from these empirical, concrete, and timestamped measures of personality traits.

Another cognitive concept that may be used with the proposed framework is the working alliance concept. The therapeutic working alliance, representative of the relationship or bond between a patient and his/her therapist, is an important predictor of the outcome of the psychotherapy treatment. Traditionally, the working alliance is estimated from a set of scoring questionnaires in an inventory that both the patient and the therapists fill out. The alliance involves several cognitive and emotional components of the relationship between these two agents, including the agreement on the goals to be achieved and the tasks to be carried out, and the bond, trust, and respect to be established over the course of the therapy.

The proposed approaches of Framework 2 quantify different cognitive properties (e.g., personality traits or working alliance) by projecting each turn in an interactive event (e.g., a psychotherapy session) onto the representation of clinically established psychiatric inventories, using language modeling to encode both turns and inventories. This framework can be used not only to quantify the overall degree of the cognitive properties used but also to identify granular patterns its dynamics over shorter and longer time scales. The proposed approaches can also be used as a companion tool to provide feedback to a therapist and to augment learning opportunities for training therapists.

Framework 2 analyze dialogue data and/or resultant output produced from the dialogue data, using, for example, machine learning systems (e.g., based on neural network architectures). The proposed frameworks can classify patients into categories using a combination of architectures that includes, for example, DenseNet, Convolutional Neural Net (CNN), Recurrent Neural Net (RNN), and attention-based Transformers. During training of the machine learning system(s) that may be used to implement the frameworks proposed herein, the framework's controller can randomly select a subsection of training data to balance the training dataset used to train the machine learning system to identify the proper diagnosis group. The framework proposed herein can be used as a tool by mental health professionals to improve patient diagnosis accuracy.

The technological framework described herein learns to predict a patient's psychiatric diagnosis by learning patterns from existing psychotherapy session transcripts (and/or other types of conversational transcripts between the patient and other individuals, not just the therapist). The transcribed conversations are transformed into a series of learnable traits which are mapped to the likelihood of psychiatric diagnoses. The proposed framework may include a platform that includes one or more of: a diagnosis tool with respect to one or more general categories e.g., (anxiety, depression, schizophrenia, and suicidal intents, etc.), a tool to indicate whether a patients' mood is stable or in flux, an in-office tool to aid psychiatrists, and/or a mobile platform or chat-bot which could be used to monitor patients outside of their psychiatric therapy sessions.

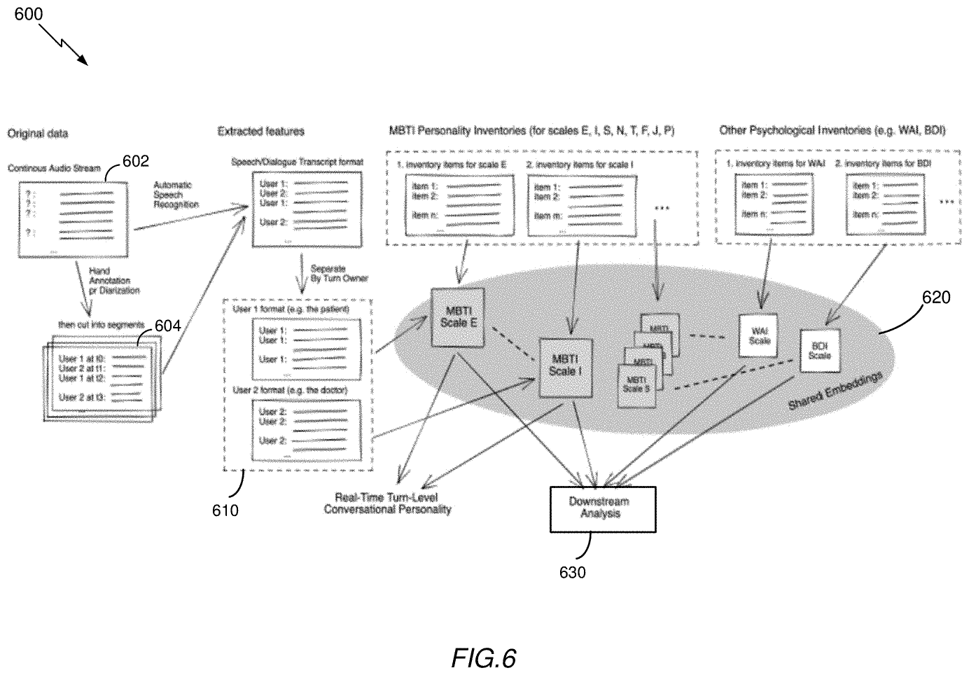

With reference to FIG. 6, a schematic diagram of an example proposed analytic framework 600 to infer cognitive anchors according to one or more psychological inventories (e.g., automatically annotating therapeutic affinity according to the working alliance inventory) using conversational data (e.g., transcript data obtained for interactive events, such as psychotherapy sessions between a patient and a therapist) is shown.

As depicted in FIG. 6, full records 602 of a patient, or a cohort of patients having the same or similar condition, are obtained, and are used to derive speech segments 604 that are used in the analysis applied to the conversational data. For example, transcribed dialogue data (corresponding to the patient-therapist psychotherapy session) can be used as-is, or can be truncated into shorter segments based on timestamps or topic turns. As discussed above in relation to Framework 1, when a transcript is provided as a paired dialogue (between a patient and a therapist), features in the transcript can be extracted, by an extractor 610, in one of several ways. In a first example extraction strategy, the full set of dialogue pairs is provided as input to a processing engine (i.e., what is processed are dialogue pairs, whether broken down to single turn segments, or to longer multi-turn segments). A second example extraction strategy is one in which only the patient's transcribed content is used as the input data. In a third strategy, only the other speaker's (therapist's) transcribed content is used as input data (either one turn at a time, or multi-turns at a time). Of course, other sequential data extraction strategies may be used, e.g., grouping patient's content from multi-turns together, grouping the other speaker's content from multi-turns together, and processing the patient's and speaker's content groupings separately. The different feature extraction formats (strategies) all have their pros and cons. The dialogue format contains all the information obtained from a psychotherapy session, but the intent within the sentences comes from multiple individuals, so the data might result in confusion or ambiguity. The patient format contains the full narrative of the patient, which is usually more coherent, but is only part of the story. The other speaker format, which can provide accurate semantic labels of what the patient feels, can be informative, but may also be sometimes too simplistic.

Having derived the speech segments/features, the speech segments can be compared to, for example, a working alliance inventory (or some other affinity inventory or ontology) transformed into embeddings. The comparison is performed by a machine learning comparator (schematically represented as ellipse 620) that is trained to produce embeddings (vectors) from conversational input and compare those embeddings to vector representations (derived from the same machine learning models) for a particular psychological inventory. In some examples, a different machine learning embedding-producing model may be used for different inventories (or even for individual inventory groupings or scales within a particular inventory). For example, the Working Alliance Inventory (WAI) is a set of self-report measurement questionnaire that quantifies the therapeutic bond, task agreement, and goal agreement between a patient and a therapist. Since being launched as a 12-item version, the inventory has used parallel versions for clients and therapists with good psychometric properties and helped establish the importance of therapeutic alliance in predicting treatment outcomes. A modern version of the inventory includes 36 questions, and a participant (be it a patient or a therapist) is asked to rate each item on the corresponding questionnaire on a 7-point scale (1=never, 7=always). The WAI aims to (1) measure alliance factors across all types of therapy, (2) document the relationship between the alliance measure and the corresponding theoretical constructs underlying the measure, and (3) relate the alliance measure to a unified theory of therapeutic change.

Operationally, the goal is to derive from these 36 items (or some other quantity of items) three alliance scales: the task scale, the bond scale, and the goal scale. These scales measure the three major themes of psychotherapy outcomes: (1) the collaborative nature of the patient-therapist relationship, (2) the affective bond between therapist and patient, and (3) the therapist's and patient's capabilities to agree on treatment-related short-term tasks and long-term goals. The scores corresponding to the three scales come from a key table which specifies the positivity or the sign weight to be applied on the questionnaire answer when summing in the end. The full scale is simply the sum of the scores of the three scales. The key table is like a weighting matrix that specifies the directionalities of the scales.

As noted, another inventory of cognitive inventory that can be used to analyze conversational transcripts for the patient is the Myers-Briggs type indicator (MBTI) inventory. As one of the most widely used measures of normal personalities, MBTI uses a psychological questionnaire inventory that includes forced binary choice questions to assess an individual's propensities in four function and attitude pairs based on the classical Jungian psychology (Extraversion-Introversion, Sensing-Intuition, Thinking-Feeling, and Judging-Perceiving). It has been posed that individuals with similar personality types in these four functional scales would adopt a similar perspective for interacting with the others and the world.

Operationally, the goal for this inventory is to derive from 70 inventory items eight personality scales: Extraversion (E), Introversion (I), Sensing (S), INtuition (N), Thinking (T), Feeling (F), Judging (J), and Perceiving (P). These scales measure the different major themes of the personality. The score corresponding to the three scales comes from a key table which specifies the weight or the sign weight to be applied on the questionnaire answer when summing in the end for each scale. After obtaining the eight scales, four binary labels are extracted by comparing the value of the score in these paired scales: Extraversion—Introversion (E/I), Sensing—INtuition (S/N), Thinking—Feeling (T/F), and Judging—Perceiving (J/P). Finally, combining the resultant values together yields a 4-letter MBTI code.

An example cognitive inference with inventory binding (CIIB) process that is applied to the psychotherapy data is the following.

Cognitive Inference with Inventory Binding (CIIB) process

| | |
|---|---|
| 1: | for i = 1,2, ^{...} , T do |
| 2: | Automatically transcribe dialogue turn pairs (S^{p}ᵢ, S^{t}ᵢ) |
| 3: | for (I^{p}ⱼ, I^{t}ⱼ) ∈ inventories (I^{p}, I^{t}) do |
| 4: | Score W^{Pi}j = similarity(Emb(I^{p}ⱼ),Emb(S^{p}ᵢ)) |
| 5: | Score W^{ti}ⱼ = similarity(Emb(I^{t}ⱼ),Emb(S^{t}ᵢ)) |
| 6: | end for |
| 7: | end for |

In the above example CIIB process, dialogue data is transcribed into pairs of turns. Each patient response turn is denoted as S^{p}ᵢ, followed by a counterpart speaker (therapist, friend, relative, another patient) response turn S⁶ᵢ. The response turns are treated as dialogue pairs. The cognitive inventories questionnaires also come in pairs: Iₚ for the patient (or client), and Iₜ for the therapist (or whoever the other speaker is). In the Working Alliance example, each inventory (dictionary/ontology of concepts) may comprise 36 statements (that are descriptive or representative of the state of the patient-therapist relationship). In the above example, the dialogue turns (extracted features) and the concepts in the inventories are transformed into an embedding (vector) space using, for example, a machine learning engine (e.g., implemented with a long short-term memory, or LSTM, neural network configuration, or using some other machine learning architecture). The transformations embed the speech segment features and the inventory concepts into deep sentence or paragraph embeddings. In principle, any sentence or paragraph embeddings can help characterize the dialogue turns and inventories. In the example framework implementations described herein, two deep embeddings were used. The first was the Doc2Vec embedding (a popular unsupervised learning model that learns vector representations of sentences and text documents). This embedding improves upon the traditional bag-of-words representation by utilizing a distributed memory that remembers what is missing from the current context. The other embedding that was used was the SentenceBERT, which modifies a pretrained BERT network by using Siamese and triplet network structures to infer semantically meaningful sentence embeddings. With these two deep embeddings, the turn-level entries (either the dialogue turn in the transcripts, or the statement items in the working alliance inventories) were embedded (transformed) into vectors of 300 or 384 dimensions.

Having transformed the speech segments and inventories into the embedding space, a similarity score (e.g., cosine similarity) between the embedding vectors of the turns (speech segment features) and its corresponding inventory vectors is computed. For the WAI example, for each turn (either by patient or by therapist), a 36-dimension working alliance score is derived (the dimension can be larger or smaller, depending on the size of the inventory used). The similarity score, representative of the closeness, similarity, or relevance of statements made by the patient or therapist to a pre-determined dictionary/inventory of concepts, provides interpretable information that can be further analyzed (e.g., by a downstream analysis process 630 which may also be based on a machine learning implementation).

There are several downstream tasks and user scenarios that can be used in the proposed analytical frameworks. For example, the resultant similarity scores (or some other output that was produced, e.g., weighted topic labels) inform whether the therapy is going in the right direction, whether the patient is entering some bad mental state, or whether the therapist should adjust his or her treatment strategies. The downstream stage/section can be implemented as an intelligent AI assistant to communicate to the therapist (during or after a psychotherapy session) appropriate output. For example, if the resultant output (similarity scores) indicates, upon analysis by a downstream engine, to constitute an emergency (e.g., the patient's working alliance similarity scores are indicative of suicidal tendencies), the therapist can be alerted through a notification sent to a computing device (e.g., a mobile computing device) associated with the therapist.

In some embodiments, Framework 2, as described herein, may include a real-time AI system to conduct sentence-level quality assurance of conversational alignment based on speaker-diarized dialogues transcribed from automatic speech recognition of a continuous audio stream(s). In such embodiments, the framework may utilize an online registration-free speaker-diarization engine to perform separation of speech utterances of multiple speakers in the conversations, that learns from user feedback. A preferable AI engine for a realistic speaker diarization system should, (1) not require user registrations, (2) allow new users to be registered into the system real-time, (3) transfer voiceprint information from old users to new ones, and (4) be up and running without pretraining on large amount of data in advance. Requirement (4) introduces an additional caveat that the labeling of the user profiles happens purely on the fly, trading off models pre-trained on big data with the user directly interacting with the system by correcting the agent as labels. To tackle these challenges, in an example implementation the BerlinUCB, an online semi-supervised learning bandit algorithm to do diarization, was used by treating this problem as an interactive online learning problem with cold-start arms and episodically revealed rewards (the user can either reveal no feedback, approving the agent by not intervening, or correcting the agent). For each episodes without feedbacks, a self-supervision process assigns a pseudo-action upon which the reward mapping is updated. Finally, the framework generates new arms by transferring the learned arm parameters for similar profiles given the user feedbacks.

Thus, in some embodiments, a dialogue data analysis system is provided that includes one or more memory devices to store processor-executable instructions and dialogue data relating to one or more events involving a patient and at least another speaker, and a processor-based controller, coupled to the one or more memory devices. The processor-based controller is configured, when executing the processor-executable instructions, to transform one or more patient speech segments and one or more speech segments of the at least other speaker, representative of the dialogue data, into representations in a vector space to produce one or more vectored patient representations and one or more vectored speaker representations. The processor-based controller is further configured to determine one or more patient similarity scores between the one or more vectored patient representations and one or more vectored representations of a set of semantic elements in one or more inventories of cognitive properties, determine one or more speaker similarity scores between the one or more vectored speaker representations and one or more vectored representations of another set of semantic elements in the one or more inventories of cognitive properties, and determine based on the one or more patient similarity scores and the one or more speaker similarity scores a psychiatric assessment for the patient.

In some embodiments, a non-transitory computer readable media is provided that includes computer instructions executable on a processor-based device to transform one or more patient speech segments and one or more speech segments of at least another speaker, representative of spoken dialogue between a patient and the at least other speaker, into representations in a vector space to produce one or more vectored patient representations and one or more vectored speaker representations. The computer instructions further cause the processor-based device to determine one or more patient similarity scores between the one or more vectored patient representations and one or more vectored representations of a set of semantic elements in one or more inventories of cognitive properties, determine one or more speaker similarity scores between the one or more vectored speaker representations and one or more vectored representations of another set of semantic elements in the one or more inventories of cognitive properties, and determine based on the one or more patient similarity scores and the one or more speaker similarity scores a psychiatric assessment for the patient.

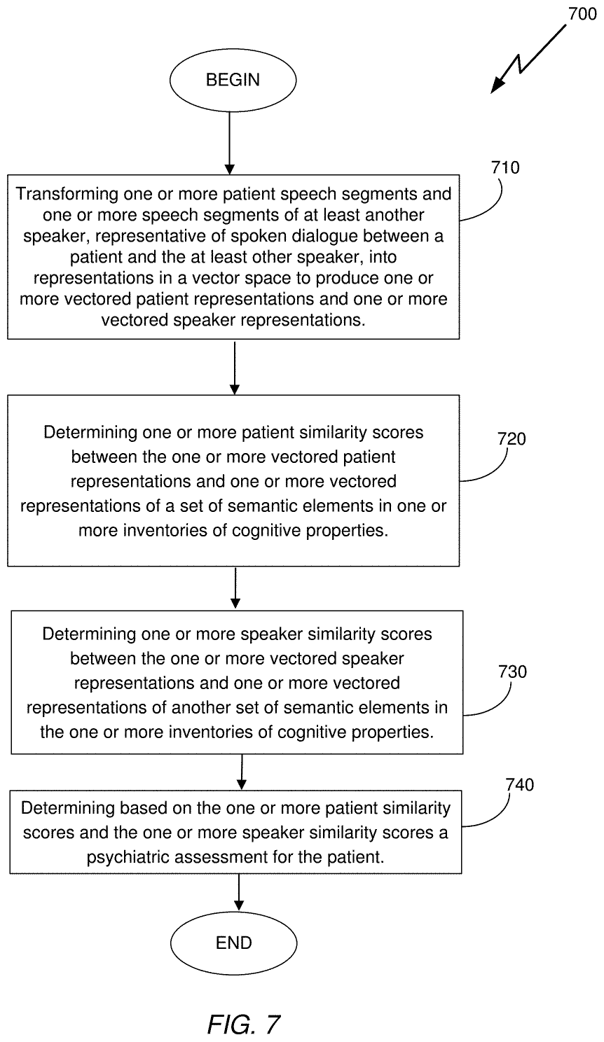

With reference next to FIG. 7, a flowchart for a procedure 700 for analyzing dialogue data is provided. The procedure 700 includes transforming 710 one or more patient speech segments and one or more speech segments of at least another speaker, representative of spoken dialogue between a patient and the at least other speaker, into representations in a vector space to produce one or more vectored patient representations and one or more vectored speaker representations. In some embodiments, the at least other speaker may include one or more of, for example, one or more family members of the patient, one or more friends of the patients, one or more therapists, and/or one or more other patients participating in one or more group therapy sessions. In some examples, transforming the one or more patient speech segments and the one or more speech segments of the at least other speaker may include transforming the speech segments using a neural network that includes a word embedding layer. In such embodiments, the word embedding layer may include one of, for example, a Word2vec layer, or a Bidirectional Encoder Representations from Transformers (BERT) layer.

With continued reference to FIG. 7, the procedure 700 further includes determining 720 one or more patient similarity scores between the one or more vectored patient representations and one or more vectored representations of a set of semantic elements in one or more inventories of cognitive properties, determining 730 one or more speaker similarity scores between the one or more vectored speaker representations and one or more vectored representations of another set of semantic elements in the one or more inventories of cognitive properties, and determining 740 based on the one or more patient similarity scores and the one or more speaker similarity scores a psychiatric assessment for the patient. In some embodiments, the psychiatric assessment for the patient may include one or more of, for example, mental state of the patient, a therapy adjustment recommendation, or a trajectory of therapy for the patient.

In various examples, the procedure 700 may further include deriving the one or more vectored representations of the set of semantic elements and the one or more vectored representations the of the other set of semantic elements by transforming, into the vector space, therapy alliance semantic statements defining a Working Alliance Inventory (WAI) dataset, with the therapy alliance semantic statements being representative of therapeutic alliance of patient-perspective characteristics and therapist-perspective characteristics of one or more psychotherapy sessions. In such embodiments, the patient-perspective characteristics and the therapist-perspective characteristics may represent one or more of, for example, collaborative nature of the patient's and a therapist's relationship, an affective bond between the therapist and the patient, and/or capabilities of the patient and the therapist to agree on treatment-related short-term tasks and long-term goals.

The procedure 700 may include, in some embodiments, deriving the one or more vectored representations of the set of semantic elements and the one or more vectored representations the of the other set of semantic elements by transforming, into the vector space, semantic content based on the Myers-Briggs type indicator (MBTI) inventory, with the semantic content based on the MBTI inventory being representative of personality traits and behavioral trajectories for the patient and the at least other speaker.

In some examples, the procedure 700 may further include obtaining transcript data representative of the spoken dialogue in one or more events involving the patient and the at least other speaker, and extracting from the transcript data the one or more data patient speech segments and the one or more speaker speech segments. In such examples, obtaining transcript data may include receiving multi-speaker audio data, and performing speech separation for the multi-speaker audio data to identify respective speech utterances for the patient and the at least other speaker.

Implementations of proposed Framework 2 were tested and evaluated to study their efficacy and performance. The evaluation result demonstrate that the implementations of Framework 2 outperformed a selected baseline performance despite not being pretrained with any labels.

More particularly, the Kaggle Myers-Briggs Personality Type Dataset is a classification dataset that comprises over 8600 users tested for their MBTI codes. This data was collected through the PersonalityCafe forum, an online platform with a large selection of MB TI-validated users and their online presence in this forum. The feature for each MBTI prediction label (as a 4-letter code) is a section of each of the last fifty (50) things the users have posted. Since the goal for the implementations of Framework 2 was not to find the best classification model for personality prediction, the evaluations were focused on showing that the unsupervised inference implementation can be predictive of the underlying personality label, instead of designing a best-performing supervised deep learning architecture. The baseline approach selected for the evaluation was one that included: (1) a balancing approach to correct for the severe imbalances innate in the dataset, (2) a selective word removal pipeline that crops out stop words, weird texts, website links and other non-meaningful terms, (3) a standard lemmatization and tokenization framework to make the words generalizable across languages and tenses, and (4) a padding step to boost the feature treatment of their classifier. The classifier consists of a deep embedding followed by a recurrent and dense architecture, and yielded a good classification result.

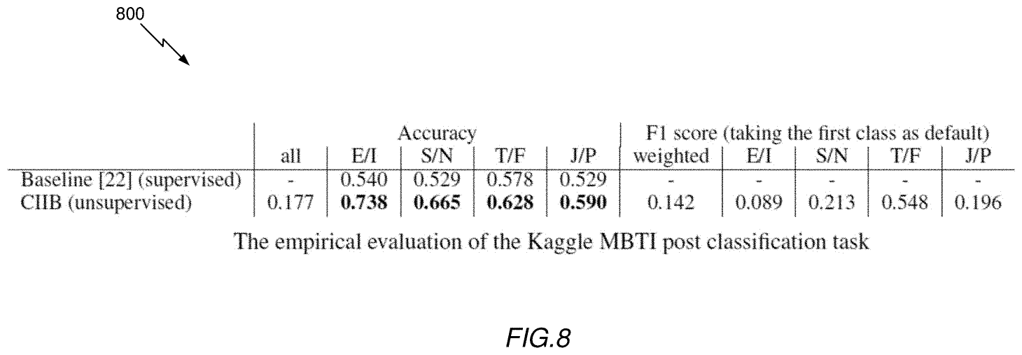

The implementations of Framework 2 were based on unsupervised approach. As a result, there was no training on the label involved in this task. To avoid unnecessary efforts, no preprocessing steps were performed (in contrast to the baseline approach). In other words, the raw, unfiltered and uncleaned text is fed directly into the evaluated implementations of Framework 2, which uses an out-of-the-box document embedding (e.g., the Sentence Bert in this case). The inference score of the four scales of MBTI is then computed (Extraversion—Introversion, Sensing—INtuition,-Thinking—Feeling, Judging—Perceiving). The bigger scores in these four scales are treated as the inferred label. The labels of the four scales are then combined to get a 4-letter MBTI. FIG. 8 includes a table 800 with the empirical evaluation results of the Kaggle MBTI post classification task. The results provided in the table 800 show that even when the unsupervised approach, implemented by the Framework 2, is not trained with any label, it yields a better performance than the baseline supervised approach. The distinctions of on the ExtraversionIntroversion and Sensing—INtuition spectrums are especially prominent, considering these cognitive properties are mostly implicit in natural language. Other metrics are also reported.

Next, a real-world dataset of doctor-patient conversations was analyzed with the implementations of Framework 2. It was demonstrated that the approaches realized by Framework 2 help parse useful insights for clinical psychiatry applications. In the evaluation conducted, the Alex Street Counseling and Psychotherapy Transcript Dataset, comprising transcribed recordings of over 950 therapy sessions between multiple anonymized therapists and patients, was used. This multi-part collection includes speech-translated transcripts of the recordings from real therapy sessions, 40,000 pages of client narratives, and 25,000 pages of reference works. These sessions belong to four types of psychiatric conditions: anxiety, depression, schizophrenia and suicidal. Each patient response turn S^{p}ᵢ, followed by a therapist response turn S^{t}ᵢ, was treated as a dialogue pair. In total, these materials included over 200,000 turns together for the patient and therapist and provide access to the broadest range of clients for linguistic analysis of the therapeutic process of psychotherapy.

The full session transcript was annotated into inferred personality scales and working alliance scores time-stamped by turns. While this level of temporal resolution gives more subtlety and insights into the temporal dynamics, they can be volatile. As a result, a session-level summary statistics of these inferred variables was computed by averaging out the numerical scores and taking the majority categorical labels as the session labels. For instance, if in a conversation, the doctor speaks five turns, and has an inferred MBTI E-scale score to be [0.3, 0.2, 0.25, 0.25, 0.5] and MBTI codes of [ISTP, INFJ, INTJ, INFJ, ISFJ], then the aggregated MBTI E-scale score and MBTI label would be 0.3 and INFJ.

The clinical target of interest, the working alliance score, was explored by plotting out the pairwise distribution, colored with the session-wise MBTI labels. It was observed that the alliance scores vary across the scales. When the relationship among the scales was investigated, it was observed that the task scale positively correlated with the bond scale in both versions, while the goal scale slightly negatively correlates with the task scale in the therapist version. It was also observed that, comparing these pairwise distributions of the patient's turns with the therapist turns, the personalities were distributed differently across the working alliance spectrums. For instance, there was a larger population of INFJ detected in the therapist's working alliance scores, and seemingly splitting the center population of ENTP into two clusters. This is interesting because INFJ are known to be the “Counselor” or “Advocate” personality, and has a career path suggestion related to psychiatry.

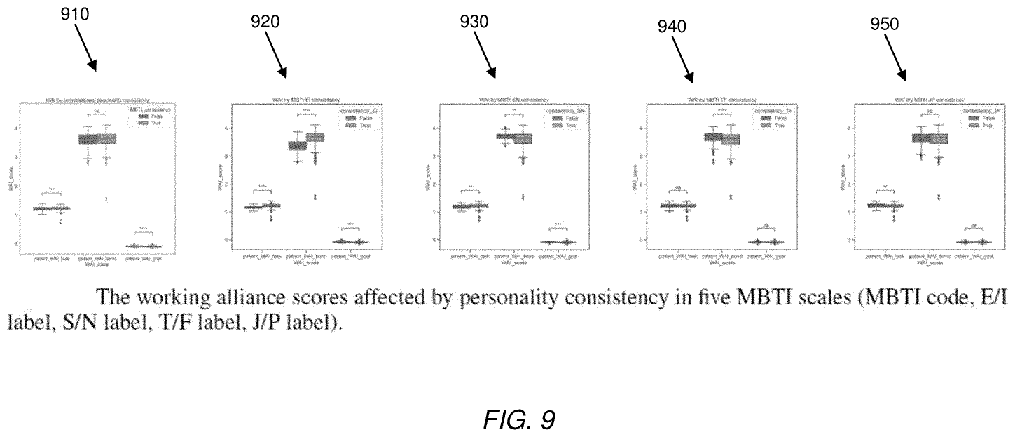

The personality consistency between the patient and the therapist was investigated. If the therapist and patient have a different personality type, they were marked as having inconsistent personalities, and vice versa. FIG. 9 includes graphs 910, 920, 930, 940, and 950 showing the session-wise working alliance scores in the three scales and their relationship to the personality consistency between the therapist and the patient. It was determined that the patient and therapist have an exact matching MBTI code that can significantly increase the patient's working alliance score in the TASK scale (p<0.001), but significantly decrease their alliance in the GOAL scale (p<0.0001). The personality consistency in binary labels, such as the Extraversion—Introversion, yields different results. The matching patient-therapist personality in Extraversion—Introversion scale can significantly improve the working alliance in TASK and BOND scales (p<0.00001), but significantly decreases their GOAL score (p<0.001). Matching Sensing—INtuition increases the TASK score (p<0.01) and decreases the BOND (p<0.01) and GOAL (p<0.001) scores. Matching Thinking—Feeling decreases the BOND score (p<0.0001). Matching Judging—Perceiving decreases the TASK score (p<0.01).

Thus, the approaches and solutions of Framework 2 combine language modeling with the knowledge and practical expertise in psychotherapy, as captured in therapy-evaluation inventories, to provide a uniquely granular representation of the evolution of the interaction of patients and therapists. The analytic approach reveals several insightful features of the personality traits of the therapist and patient, as well as their therapeutic relationship.

These features of the therapeutic dialogue can be mapped to what in psychiatry is usually called alignment and plays an important symptomatic and diagnostic role in several neuropsychiatric conditions, e.g., in relation to the hypothesis of Theory of Mind for schizophrenia. By analyzing past sessions, and eventually sessions in real time, trained therapists may be able to identify key segments of the therapy leading to breakthroughs, compounding their expertise with further causal/predictive analytic modeling, while trainees may sharpen their intuition by reading or watching annotated versions of sessions conducted by experts. Needless to say, coupled with a generative language model and further statistical optimization, it may be possible to design chatbots to engage patients in triage and emergency response. While the discussion regarding Framework 2 focused specifically on MBTI and WAI, the methodology is generic and can be extended to the broader spectrum of assessment instruments. Finally, it would be possible to refine and further validate the language-based estimation of working alliance by providing punctuated rater evaluations as inference anchors.

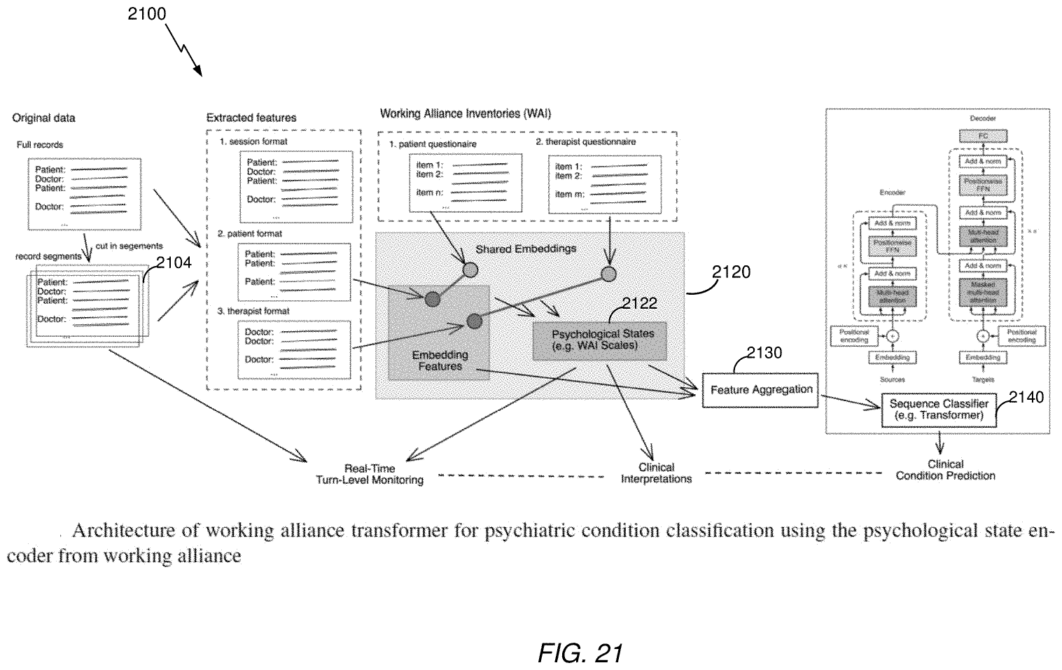

Another implementation of Framework 2 is depicted in FIG. 21, which is a diagram of an example architecture 2100 of a working alliance transformer (WAT) for psychiatric condition classification using a psychological state encoder. The WAT implementation is a transformer-based classification model to classify the psychotherapy sessions into different psychiatric conditions. The transcripts of a therapy session (and optionally the medical records of the patient and/or other types of written or oral materials), the dialogue data is separated into pairs of turns as the timestamps. The data may be separated into turns by the patient(s), by the therapist(s), or both (as a paired input). As noted, empirically, the patients' turns are usually more narrative, as they are describing themselves, while the therapists' turns are usually more declarative, as they are usually confirming the patients, or leading the conversations to a certain topic.

In the implementation shown in FIG. 21, the clinical inventory of the working alliance ((WAI) was used. The modern WAI consists of 36 questions or statements in a self-report questionnaire which measures the therapeutic bond, task agreement, and goal agreement, where the rater (i.e., the patient or the therapist) is asked to rate each statement on a 7-point scale (1=never, 7=always). This inventory is disorder-agnostic, meaning that it measures the alliance factors across all types of therapies, and provides a record of the mapping from the alliance measurement and the corresponding cognitive constructs underlying the measurement under a unified theory of therapeutic change.

The inference goal is to compute a score that characterizes the working alliance given the clinical inventory, with for instance, a feature vector of 36 dimension that correspond to the 36 alliance measure of interests in the inventory. After computing the information regarding the predicted clinical outcome with the inferred working alliance scores, this feature vector highlights a bias towards what the clinicians would care about in the psychotherapy given the metrics provided by the working alliance inventory. Nevertheless, the feature vector can be further used to potentially inform of the psychiatric condition of a given patient. Particularly, in the implementation of FIG. 21, the Working Alliance Transformer (WAT) realizes a classification model that utilizes an inference module that informs the downstream classifier where the current state is with respect to the therapeutic trajectory or landscape in the psychotherapy treatment of a patient (e.g., is this patients approaching a breakthrough? Or is he or she susceptible to a rupture of trust?)

The therapeutic information about working alliance can vary across clinical conditions, and as a result, potentially beneficial to the diagnosis and monitoring of the psychiatric disorders. The example process below outlines the classification process used under the WAT implementation.

Working Alliance Transformer (WAT)

| | |
|---|---|
| 1: | Input: a session with T turns |
| 2: | Output: a label for psychiatric condition |
| 3: | for i = 1,2, ^{...} , T do |
| 4: | Automatically transcribe dialogue turn pairs (S^{p}ᵢ, S^{t}ᵢ) |
| 5: | for (I^{p}ⱼ, I^{t}ⱼ) ∈ inventories (I^{p}, I^{t}) do |
| 6: | Score W^{Pi}j = similarity(Emb(I^{p}ⱼ),Emb(S^{p}ᵢ)) |
| 7: | Score W^{ti}ⱼ = similarity(Emb(I^{t}ⱼ),Emb(S^{t}ᵢ)) |
| 8: | end for |
| 9: | Patient feature x_{c} = concat(Emb(S^{p}ᵢ),W^{ti}) |
| 10: | Therapist feature xₜ = concat(Emb(S^{t}ᵢ),W^{ti}) |
| 11: | Full feature x = concat(xₜ, x_{c}) |
| 12: | Aggregated feature X.append(x) |
| 13: | end for |
| 14: | obtain prediction y = Transformer(X) |

During the session, the dialogue between the patient and therapist are transcribed into pairs of turns 2104. The patient turn is denoted as S^{p}ᵢ followed by the therapist turn S^{t}ᵢ, as a dialogue pair. Similarly, the inventories of working alliance questionnaires come in pairs (Iₚ for the patient, and Iₜ for the therapist, each with 36 statements). The distributed representations of both the dialogue turns and the inventories are computed with the sentence embeddings at a vector transformation engine at box 2120. The working alliance scores can then be computed as the cosine similarity between the embedding vectors of the turn and its corresponding inventory vectors. For example, as discussed herein, SentenceBERT and Doc2Vec embedding can be used as sentence embeddings for the working alliance inference. With that, for each turn (either by patient or by therapist), a 36-dimension working alliance score is obtained.

For the classification, the 36-dimension working alliance scores, computed from the current turn in the dialogue, are concatenated (at Feature Aggregation unit 2130) along, optionally, with the sentence embedding of the current turn, as the feature vector to be fed into the Transformer sequence classifier 2140.

The analytical features enabled by the working alliance inference are not only useful for the classifications investigated, but also other downstream tasks, such as predictive modeling and real-time analytics. In the implementation of FIG. 21, turns in a dialogue or monologue are fed into the sentence embedding sequentially as individual entries. Then, given the sentence embedding, each is fed into the psychological state encoder that infer the psychological or therapeutic state of the dialogue at this turn. The encoder 2122 generates a vector that characterizes the state, such as the 36-dimension working alliance scores, corresponding, for example, to the 36 working alliance inventory items. Then, the model aggregates both the sentence embedding feature vector and the psychological state vector. Since the input is fed sentence-by-sentence (or turn by turn), a turn-based sequence of combined feature vector, which is then fed into a sequence classifier. The transformer is used as a classifier for its effectiveness in various sequence-based learning tasks, and potential interpretability from its attention weights. The output of this classification model is the predicted clinical condition of this sequence. The sequence of turns used to generate a label is typically either the entirety or a segment of a session of a psychotherapy transcript.

For the implementation depicted in FIG. 21, the procedure 700 of FIG. 7 may further include deriving a feature vector based at least on the one or more patient similarity scores and the one or more speaker similarity scores, and providing the feature vector to a machine-learning sequence classifier to determine a psychological state for the patient. In addition to the scores, the feature vector may also include at least a portion of some of the computed embeddings (e.g., Emb(S^{p}ᵢ)) and/or Emb(S^{t}ᵢ)). The feature vector may be derived by combining (e.g., concatenating) at least some portions of the scores and/or embeddings.

The implementation of FIG. 21 was evaluated and tested to assess its effectiveness. The psychotherapy dataset evaluated was highly imbalanced across the four clinical conditions (495 anxiety sessions, 373 depression sessions, 71 schizophrenia sessions, and 12 suicidal sessions). To correct for this imbalance, a sampling technique was used. Instead of going through the entire training data in epochs, the models were trained in sampling iterations. In each iteration a class was randomly chosen and then one session from the class pool was randomly sampled. Before the sessions were sampled, the dataset was split into 20/80 as the test set and training set. Then during the training or the test phase, the sampling technique was performed for each iteration only in the fully separated training and test sets. Then, for each sampled session, the classification model was fed with the first 50 dialogue turns of the transcript, turn by turn, and the sequence classifier outputs a label predicting which psychiatric condition the session belongs to.

The evaluation and testing also evaluated three classifier backbones. The first one was the classical transformer model. For the multi-head attention module, the number of heads was set to be 4 and the dimension of the hidden layer was set to be 64. The dropout rates for the positional encoding layer and the transformer blocks were both set to be 0.5. The second sequence classifier was single-layer Long Short-Term Memory (LSTM) network with 64 neurons. The third sequence classifier was a single-layer Recurrent Neural Network (RNN) with 64 neurons.

For each of the three classifiers, three types of features were compared as the input was fed into the sequence classifier component. The first one, the working alliance embedding, was the concatenated feature vector of both the sentence embedding vector and the psychological state vector (e.g., 36-dimension inferred working alliance scores). The second type of feature, the working alliance score, was an ablation model which only uses the state vector (the working alliance score vector). The third type of feature, the embedding, was the baseline which only uses the sentence embedding vector directly. In other words, the working alliance score introduces the bias for WAI, while the sentence embedding does not. The working alliance embedding is the feature that combined both with concatenation. And since there were two sentence embeddings to choose from (the sentence BERT and Doc2Vec), they each had 9 models in the evaluation pool.

Other than the classifier types (Transformer, LSTM or RNN), the embedding types (SentenceBERT or Doc2Vec) and the feature types (working alliance embedding, working alliance scores, or simply sentence embedding), comparison was performed using only the dialogue turns from the patients, from the therapists (or some other speaker), and from both the patients and the therapists. In the case where the turns were used from both the patients and the therapists, that data was considered to be a pair, and those data components were concatenated as a combined feature. This is in contrast to treating them as subsequent sequences because it is believed that the therapist's response are loosely semantic labels for the patient's statements, and thus, serve different semantic contexts that should be considered side by side, instead of sequentially, which would assume a homogeneity between time steps.

Results of the evaluation and testing showed that, overall, there was an observed benefit of using the working alliance embedding as the features in Transformer and LSTM-based model architectures. Among all the models, the WA-LSTM model with working alliance embedding using only the patient turns obtained the best classification result (46%), followed by the WA-LSTM model using only the working alliance score using both turns from the patients and therapists (43.4%). This suggests the advantage of taking into account the predicted clinical outcomes in characterizing these sessions given their clinical conditions. It was also observed that the inference of the therapeutic working alliance with Doc2Vec appears to be more beneficial in modeling the patient turns than the therapist turns, while the working alliance inference using SentenceBERT appears to be advantageous in both the therapist and patient features.

During training, it was observed that among the three sequence classifier variants, the vanilla RNNs sometimes fail (which was denoted by an “F”) to learn due to exploding gradients over the long time steps (over 100 turns in each session). As a result, their predictions are at the chance level and based on their confusion matrices, they only trivially select the first class label. The LSTM networks are more stable when dealing with these long time series, but there was one failure case when it is trained on the working alliance score of the therapists' turns as its features.

Comparing the three sequential learners, the Transformer, due to the additional attention mechanism, yields a more stable learning phase. When using the SentenceBERT as its embedding, it was observed a modest benefit when training on only the patient turns, which might suggest an interference of features between the therapists' and patients' working alliance information. The transformers using the working alliance embedding, i.e., both the sentence embedding and their therapeutic states (i.e., the inferred working alliance score vector) are the best performing ones. When using the Doc2Vec as the sentence embedding, the best performing models were the transformers using some of the working alliance information from the inference module as the features. These preliminary results suggest that the inferred scores of the therapeutic or psychological state can be potentially useful in downstream tasks, such as diagnosing the clinical conditions.

### Framework 3: Supporting Psychotherapy with Real-Time Recommendations of Treatment Strategies

Another example framework that uses machine learning model to facilitate with mental health provisioning includes the implementations described herein for a recommendation system that suggests treatment strategies to a therapist during the psychotherapy session in real-time. The proposed system uses a turn-level rating mechanism that predicts the therapeutic outcome by computing a similarity score between the deep embedding of a scoring inventory, and the current speech segment (one or more sentences) that the patient is speaking. In some embodiments, the system (referred to Framework 3) automatically transcribes a continuous audio stream and separates it into turns of the patient and of the therapist using a diarization method. The dialogue pairs along with their computed ratings are then fed into a collaborative filtering mechanism where the sessions are treated as users and the topics are treated as items.

Framework 3 can be realized as a SupervisorBot, a virtual AI companion that provides real-time feedback and recommends treatment strategy to the therapists while they are conducting psychotherapy sessions with patients. Like a supervisor, SupervisorBot offers feedback and guidance that are case-dependent. Also like a supervisor, SupervisorBot has seen thousands of historical therapy sessions and case studies. The base of the proposed recommendation system relies on a rating system that evaluates how good a treatment strategy is. As the mental state of a patient can be complicated to characterize, the approaches and solutions described herein gravitate towards well-defined clinical outcomes. The working alliance is a psychological concept that has been shown to be highly predictive of the success of psychotherapy in clinical setting. It describes several important cognitive and emotional components of the relationship between these two agents in conversation, including the agreement on the goals to be achieved and the tasks to be carried out, and the bond, trust, and respect to be established over the course of the dialogue. Framework 3 uses a Reinforced Recommendation model for Dialogue topics in psychiatric Disorders (R2D2), which is believed to be the first ever recommendation system of dialogue topics proposed for the psychotherapy setting. It transcribes the session in real-time, predicts the therapeutic outcome as a turn-level rating, and recommends treatment strategy that is best for the current context and state of the psychotherapy. It is the first step to solving the global issue of mental health by augmenting the treatment and education of clinical practitioners with a recommendation system of therapeutic strategy.

In the proposed analytic framework, a continuous audio stream is fed into the system. Speaker diarization is then performed. In some examples, online speaker diarization may be performed using BerlinUCB, which is an online semi-supervised learning bandit algorithm to perform diarization. The BerlinUCB can separate the input stream into patient and therapist turns. Next, after obtaining diarization output data, the quality assessment setting is configured by specifying a proper inventory (ontology). For example, the Working Alliance Inventory (WAI), also discussed above, is a set of self-report measurement questionnaire that quantifies the therapeutic bond, task agreement, goal agreement, may be used. Operationally, the goal is to derive from a set of WAI items (e.g., 36 items) three alliance scales: the task scale, the bond scale, and the goal scale. These scales measure the three major themes of psychotherapy outcomes: (1) the collaborative nature of the dialogue participants' relationship, (2) the affective bond between them, and (3) their capabilities to agree on treatment-related short-term tasks and long-term goals. The score corresponding to the three scales can be derived from a key table which specifies the positivity or the sign weight to be applied on the questionnaire answer when summing in the end. The full scale is simply the sum of the scores of the three scales. The key table is like a weighting matrix that specifies the directionalities of the scales.

Thus, briefly, given the audio stream for a given user, the diarized audio stream is transcribed (e.g., using a standard or customized automatic speech recognition module). The dialogue turns and the inventories are embedded with deep sentence or paragraph embeddings (e.g., using SentenceBERT), and the cosine similarity between the embedding vectors of the turn and its corresponding inventory vectors are computed. With that, for each turn (by patient and/or by therapist), a 36-dimension working alliance score is computed, and may be saved in a relational database.

The framework is configured to recommend “items” (e.g., using a trained machine learning system based, for example, on a neural network implementation) which are treatment strategies. In this example, these strategies are represented as topics that the therapist should initiate or continue for the next turn. Additional actionable items that the recommendation system can identify may include one or more, for example, strategies for distracting the patient, telling a joke (or other suggestions for putting the patient at ease), apologizing, interrupting the conversation, talking about person X, having the therapist share his/her own experience, performing some recommended mental exercise, etc.

The same approach can be extended to more complex and nuanced treatment suggestions. For instance, in the ABC approach of cognitive behavioral therapy (CBT), the proposed framework can suggest a belief (B) to guide the patients to better understand the causality between the activating event (A) and its consequence (C).

Given a large text corpus of many psychotherapy sessions, topic modeling is performed to extract the main concepts discussed in the psychotherapy. The Embedded Topic Model (ETM) may be used (ETM was shown to create the most diverse concepts in psychological corpus). One can also adopt a symbolic approach to the topic modeling to gain further insights into the causalities and relationships between these topical concepts. The recommendation system subsequently pairs these “items” with the “users” and “contents”, which in the present example, could be the patientlD, his or her previous turns, their aggregated formats, and other meta data. For instance, within each session there exists many pairs of turns that belong to the same “user”. However, one can also assign all turns to one clinical label, or all turns related to a certain topic as one “user”. Lastly, the “ratings” may be the patient's inferred alliance scores predictive of the therapeutic outcomes.

The proposed framework goes beyond just merely annotating and analyzing the natural language and speech data from the users, and can actually give actionable suggestions (“critical decision making”). The proposed framework provides real-time feedback and recommendations to the therapist. In various examples, the recommendation system may be configured according to one of several approaches, including content-based recommendation approaches, session-based approaches, collaborative filtering approaches, reinforcement learning approaches, etc. While session-wise recommendations are useful, real-time recommendations can pinpoint the breakthrough and rupture points in a therapy with more actionable and correctable resolutions.

In some embodiments, the recommendation system used may be implemented according to a deep reinforcement learning recommendation approach. Particularly, a reinforcement learning environment is formulated such that a recommendation agent takes an action by recommending a strategy (e.g., discussion topic). Subsequently, the therapist may take that suggestion into account when interacting with the patient. The dialogue interaction, in turn, includes a quality evaluation mechanism (say, the therapeutic working alliance score). This serves as a reward to the recommendation agent to update its weights. In the meantime, the state is progressed to the next therapeutic state.

Several reinforcement learning (RL) processes/algorithms may be used in the implementations described herein. One such process, based on the deterministic policy gradient in an actor-critic architecture, is the Deep Deterministic Policy Gradients (DDPG) process that is a model-free procedure for continuous action spaces, and has been shown to successfully learn policies end-to-end. Another possible RL process that may be used is the Twin Delayed DDPG (TD3) that builds on a Double Q-Learning approach, and provides a solution to correct for an overestimated value issue to yield more competitive results in various game settings.

Since online data collection of RL models are usually time consuming, in real world industrial setting these models are sometimes trained using previously collected data. As a result, there is a growing popularity of offline reinforcement learning approaches. Among those approaches is the Batch Constrained Q-Learning (BCQ) approach that implements a continuous control deep RL algorithm that yields competitive results in off policy evaluations by restricting the agent's exploration in the action space.

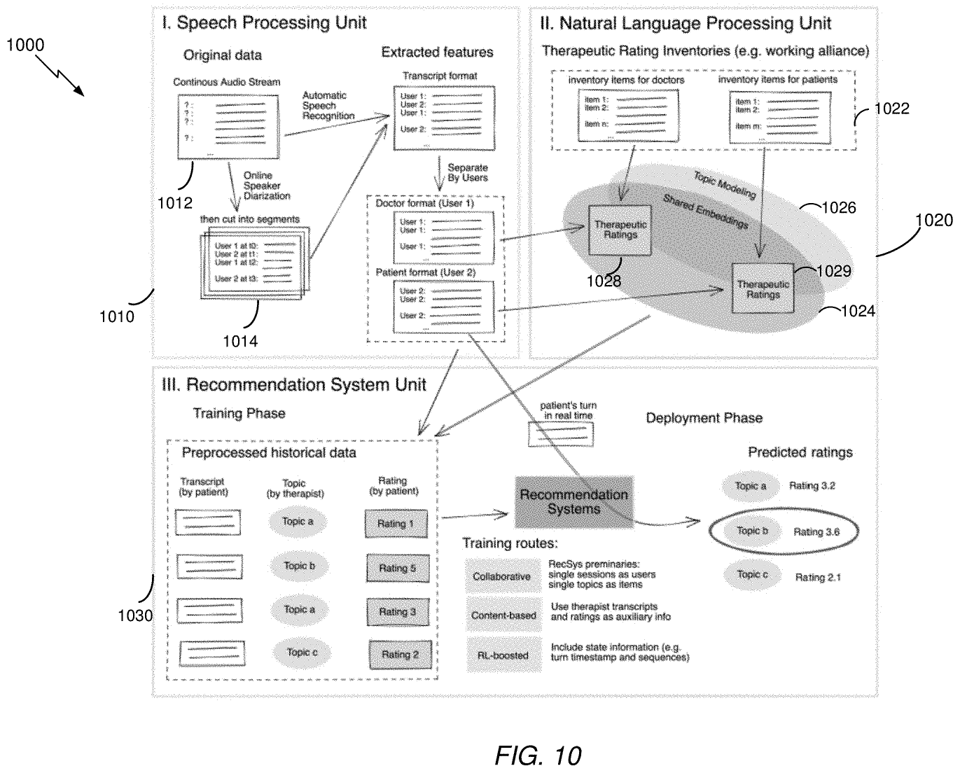

With reference to FIG. 10, a schematic diagram of a recommendation system 1000 is shown. The system 1000 includes three main components: I) a speech processing system 1010 to process audio data into transcribed written transcript data that is then diarized to separate the transcript data into data extracts (and optionally into data turns), II) a natural language Processing Unit 1020 that processes and analyzes the transcript data extracts to derive semantic analysis data (e.g., in the form of vector representations), and III) a recommendation system unit 1030 (recommendation engine) that determines recommendations responsive to the semantic analysis data.

As a preliminary step, speaker diarization is performed by training the system 1000 for a few rounds by interacting with sparse feedback from the user. As noted with respect to Framework 2, BerlinUCB, which is an online semi-supervised learning bandit algorithm to perform diarization that separates audio into dyads of doctor-patient (which are then transcribed into natural language turns for real-time downstream analyses), may be used.

After obtaining a relatively well diarization result, the quality assessment can be configured by specifying a proper inventory. For example, the Working Alliance Inventory (WAI), which is a set of self-report measurement questionnaire that quantifies the therapeutic bond, task agreement, and goal agreement can be used. As noted, operationally, the goal is to derive three alliance scales: the task scale, the bond scale, and the goal scale. These scales measure the three major themes of psychotherapy outcomes: (1) the collaborative nature of the dialogue participants' relationship, (2) the affective bond between them, and (3) their capabilities to agree on treatment-related short-term tasks and long-term goals. The score corresponding to the three scales comes from a key table which specifies the positivity or the sign weight to be applied on the questionnaire answer when summing in the end. The full scale may simply be the sum of the scores of the three scales. The key table is like a weighting matrix that specifies the directionalities of the scales.

Thus, given an audio stream 1012 for a given user, the audio stream is diarized and transcribed with automatic speech recognition module to produce speech segments 1014. Dialogue turns (determined from the speech segments) and terms of the selected inventory(ies) 1022 are transformed (embedded) into vector representations using a deep sentence or paragraph embeddings engine, e.g., SentenceBERT (the transform engines are schematically represented as ellipse 1024). Once transformed, the similarity (e.g., cosine similarity) between the embedding vectors of the turn and its corresponding inventory vectors is computed. With that, for each turn (either by patient or by therapist), a 36-dimension working alliance score is computed, which may be stored into a relational database.

The system 1000 of Framework 3 additionally produces topic modeling as recommendation items. Here, the “items” the system recommends are treatment strategies. In the example implementations described herein, these strategies are represented as a topic, generated in response to the input of current (or preceding) turn, that the therapist should initiate or continue for a next turn. Given a large text corpus of many psychotherapy sessions, topic modeling is performed by a machine learning topic modeling engine 1026 (which may be implemented similarly to the topic modeling unit/engine 120 of FIG. 1) to extract the main concepts discussed during the psychotherapy session. For example, the Embedded Topic Model (ETM) can be used to create a diverse concepts in psychological corpus. One can also adopt a symbolic approach to the topic modeling to gain further insights into the causalities and relationships between these topical concepts. In the implementations of Framework 3, each turn is annotated with its most likely topic, e.g., from a dictionary of topics. One example of a topics dictionary could include topic 0 corresponding to figuring out, self-discovery and reminiscence, topic 1 is about play, topic 2 is about anger, scare and sadness, topic 3 is about counts, topic 6 is about explicit ways to deal with stress, such as keeping busy and reaching out for help, topic 7 is about numbers, topic 8 is about continuation and keep doing).

“Items” (e.g., treatment strategy recommendation on a turn-by-turn basis) are paired with “users” and “contents”, which in the examples of Framework 3 would be the patientlD, his or her previous turns, their aggregated formats, and other meta data. For instance, it may be known that within each session there are many pairs of turns, and that they would belong to the same “user.” However, one can also assign all turns to one clinical label, or assign all turns related to a certain topic to one “user.” In evaluations and testing performed on the implementations of Framework 3 (see detailed discussion below), session ids were chosen as the “users.” Lastly, the “ratings” refers to participants (patients and/or therapists) inferred inventory (e.g., alliance) scores predictive of the therapeutic outcomes (these “ratings” outputs are represented as boxes 1028 and 1029 in FIG. 10).

With the “users,” “items,” “contents,” and “ratings” having been determined, the recommendation engine can be easily crafted with content-based and collaborative filtering. Because session turns are sequential and can specify a state or timestamp, it might be suitable for reinforcement learning (RL) and session-based approaches which can be neuroscience or psychiatry-inspired to provide further interpretable clinical insights.

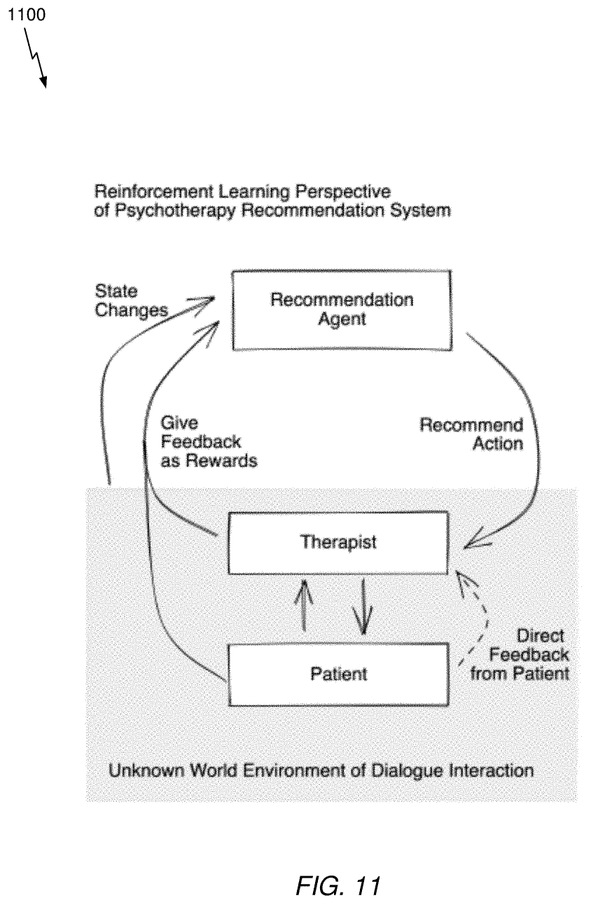

With reference now to FIG. 11, a schematic diagram of an example Reinforcement learning framework 1100 for the psychotherapy recommendation system is shown. The reinforcement learning environment is formulated such that the recommendation agent takes an action by recommending a strategy (say, a discussion topic). The therapist will interact with the patient taking that suggestion into account. The dialogue interaction, in turn, has a quality evaluation of some sort (say, the therapeutic working alliance score). This serves as a reward to the recommendation agent to update its weights. In the meantime, the state is progressed to the next therapeutic state(s).

For the reinforcement learning framework 1100, three deep RL processes are considered. Based on the deterministic policy gradient in an actor-critic architecture, the Deep Deterministic Policy Gradients (DDPG) is a model-free process for continuous action spaces, and can successfully learn policies end-to-end. Building upon the Double Q-Learning, Twin Delayed DDPG (TD3) is a similar solution that is configured to correct for the overestimated value issue, and yields more competitive results in various game settings.

As the online data collection of RL models are usually time consuming, in real world industrial setting, these models are usually trained using previously collected data. As a result, there is a growing popularity of offline reinforcement learning methods. Among them, Batch Constrained Q-Learning (BCQ) is a continuous control deep RL algorithm that yields competitive results in off policy evaluations by restricting the agent's exploration in the action space.

Accordingly, in various examples, a dynamic recommendation system is provided that includes a receiver module to obtain audio data for a patient-therapist dialogue session, and convert least part of the audio data into a current speech segment, and a processor-based controller, coupled to the one or more memory devices. The controller is configured to transform the current speech segment into a representation in a vector space to produce one or more vectored patient representations and one or more vectored therapist representations, determine one or more patient similarity scores between the one or more vectored patient representations and one or more vectored representations of a set of semantic elements in one or more inventories of cognitive properties, determine one or more therapist similarity scores between the one or more vectored therapist representations and one or more vectored representations of another set of semantic elements in the one or more inventories of cognitive properties, and determine based on the one or more patient similarity scores and/or the one or more therapist similarity scores therapist advice output to dynamically manage the dialogue session in real-time by identifying, in response to the speech segment, therapy-relevant actionable items. In some additional examples, a non-transitory computer readable media is provided that includes computer instructions executable on a processor-based device to transform the current speech segment into a representation in a vector space to produce one or more vectored patient representations and one or more vectored therapist representations, determine one or more patient similarity scores between the one or more vectored patient representations and one or more vectored representations of a set of semantic elements in one or more inventories of cognitive properties, determine one or more therapist similarity scores between the one or more vectored therapist representations and one or more vectored representations of another set of semantic elements in the one or more inventories of cognitive properties, and determine based on the one or more patient similarity scores and/or the one or more therapist similarity scores therapist advice output to dynamically manage the dialogue session in real-time by identifying, in response to the speech segment, therapy-relevant actionable items.

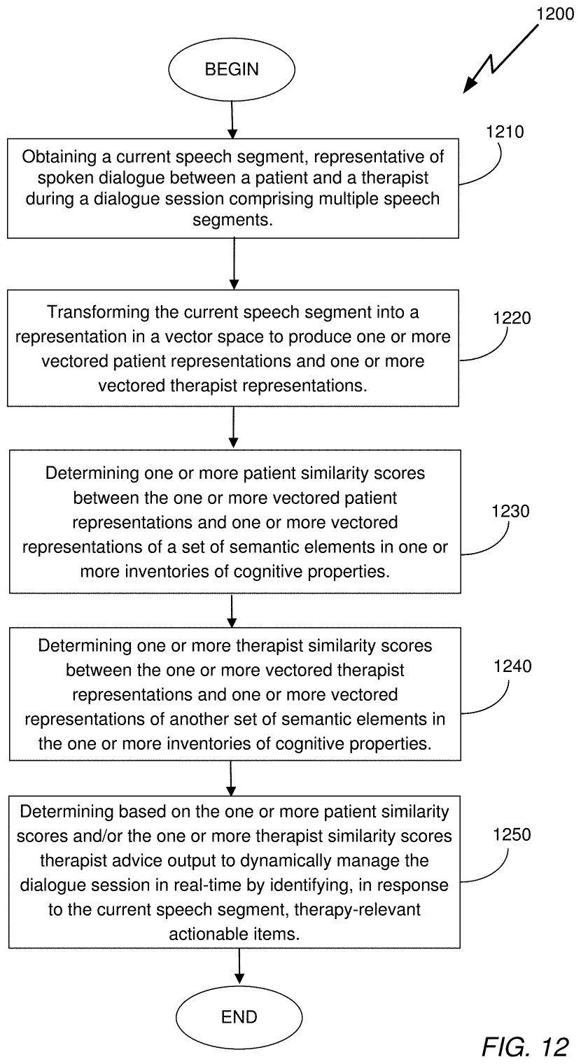

With reference next to FIG. 12, a flowchart of an example procedure 1200 for processing psychotherapy session data and making treatment strategy recommendations is shown. The procedure 1200 includes obtaining 1210 a current speech segment, representative of spoken dialogue between a patient and a therapist during a dialogue session comprising multiple speech segments, and transforming 1220 the current speech segment into a representation in a vector space to produce one or more vectored patient representations and one or more vectored therapist representations. Obtaining the current speech segment may include receiving audio data for the dialogue session, and performing speech recognition processing on the audio data to transcribe at least part of the audio data into the current speech segment. In some examples, transforming the current speech segment may include transforming the current speech segment using a neural network that includes an embedding layer. The procedure 1200 may further include deriving the one or more vectored representations of the set of semantic elements and the one or more vectored representations the of the other set of semantic elements by transforming, into the vector space, therapy alliance semantic statements defining a Working Alliance Inventory (WAI) dataset, with the therapy alliance semantic statements being representative of therapeutic alliance of patient-perspective characteristics and therapist-perspective characteristics of at least the current speech segment of the dialogue session.

With continues reference to FIG. 12, the procedure 1200 further includes determining 1230 one or more patient similarity scores between the one or more vectored patient representations and one or more vectored representations of a set of semantic elements in one or more inventories of cognitive properties, determining 1240 one or more therapist similarity scores between the one or more vectored therapist representations and one or more vectored representations of another set of semantic elements in the one or more inventories of cognitive properties, and determining 1250 based on the one or more patient similarity scores and/or the one or more therapist similarity scores therapist advice output to dynamically manage the dialogue session in real-time by identifying, in response to the current speech segment, therapy-relevant actionable items.

The therapy-relevant actionable items may include one or more of, for example, identifying additional topics to be discussed in subsequent speech segments of the dialogue session, identifying strategies for distracting the patient, identifying suggestions for putting the patient at ease, identifying strategies for re-directing the dialogue session, and/or identifying recommended mental exercises to be performed by the patient.

In some embodiments, determining the output to dynamically manage the dialogue session by identifying therapy-relevant actionable items may include determining the actionable items based on a configurable machine learning recommendation engine, and adjusting weights of the configurable machine learning recommendation engine based on quality evaluation of a subsequent action taken by the therapist in view of the actionable items determined by the machine learning recommendation engine. In such embodiments, adjusting the weights of the configurable machine learning recommendation engine may include adjusting the weights of the configurable machine learning recommendation according to one or more reinforcement learning approaches that include, for example, a deep deterministic policy gradients (DDPG) approach, a twin delayed DDPG approach, and/or a batch constrained Q-learning approach.

Implementations of proposed Framework 3 were tested and evaluated to study their efficacy and performance. The performance of the speaker diarization component was validated using the MiniVox benchmark, which showed a robust cumulative diarization accuracy at each time step. For the rating computation, since there were no ground truths, the Alex Street Psychotherapy dataset, which consists of transcribed recordings of over 950 therapy sessions between multiple anonymized therapists and patients, was analyzed. It was observed that the alliance scores significantly predict suicidality in patients and produce interesting and interpretable trajectories during the therapy sessions of different psychiatric conditions in both the alliance space and the topic space. It was also observed that the treatment strategy adopted by experienced therapists differs when facing patients of different disorders.

To evaluate the recommendation systems, the Alex Street dataset was pre-processed into a recommendation format (219,999 recommendation actions) and then split it into 95/5 train-test sets. To set up the batch training for reinforcement learning, the turns were cut into frames of 10 turn pairs and a batch size of 32. The three agents were each trained for 100 epochs, at which point their losses consistently drop and converged in a stable way. To compare the result, the Pearson's r of the recommended actions, with their corresponding ground truth actions, were computed. It was observed that BCQ was the best performing model with a correlation of 0.2843, followed by DDPG (0.2712) and TD3 (0.2192). The slight advantage might be due to the additional errors in not-offline methods introduced by extrapolation.

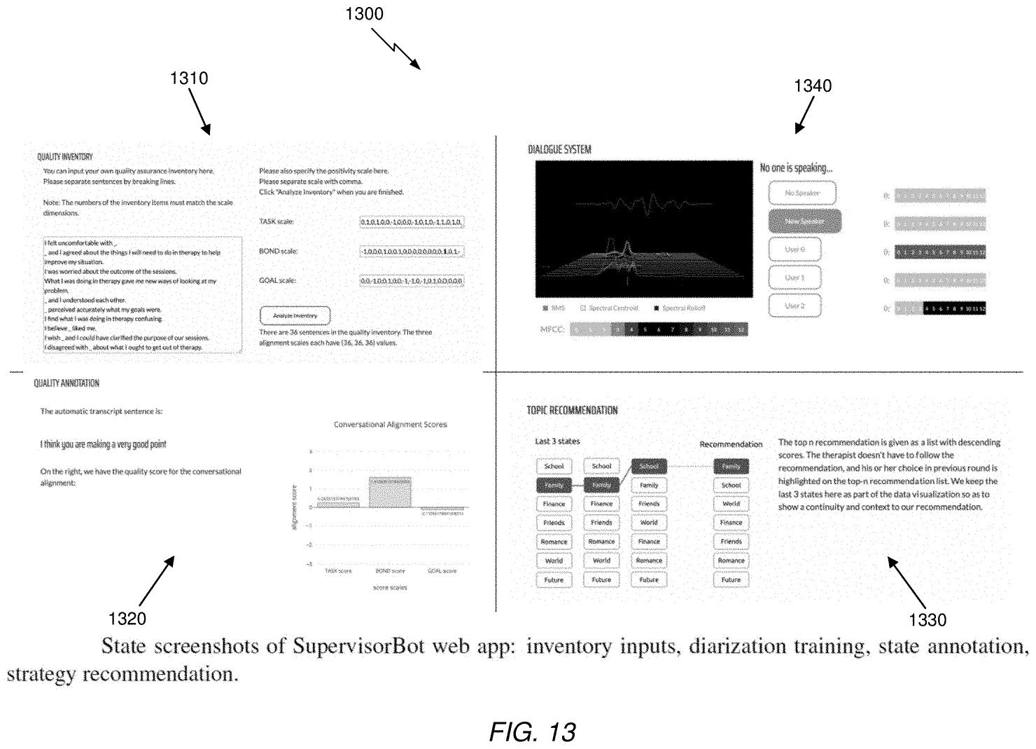

Turning to FIG. 13, state screenshots of the SupervisorBot web app system 1300 with inventory inputs, diarization training, state annotation, and strategy recommendation panels are shown. SupervisorBot is an interactive web-based system that was implemented as part of the development of Framework 3. Users were first given instructions on how to use the system. Then they were asked to input their own inventory used to evaluate the dialogue quality. In the evaluated implementations, a default inventory (the working alliance inventory) was used. The users were guided to input the score scale corresponding to each inventory item and click on “Analyze” to finalize. In the speaker diarization part, the Mel Frequency Cepstral Coefficients (MFCC) were computed and visualized in a sliding window fashion (as shown in panel 1340 of FIG. 13) given the real-time audio input from a microphone. After completing these two steps during preparation, the system 1300 commences operation, and the therapist can sit back and go on with the session. The system 1300 next moves to the annotation panel 1310, where the therapist can see that a transcript is displayed, along with an identification of who is speaking. The computed alliance scores in the three scales are also dynamically displayed (in panel 1320) in real-time according to the content of the dialogue turn. This is helpful information to assist the therapist. Panel 1330 of the system 1300 includes the recommendation guidance. The topics to choose from are ranked, and the top N are displayed. The therapist can use it as a hint and initiate his response given a top recommendation. The system will transcribe the therapist's response and highlight the topic the therapist most likely ended up choosing in the last round, and save that information as part of historical data (e.g., optionally for reinforcement learning). The system refreshes its parameters at the end of each session to fit new data.

Accordingly, as described herein, the implementations of Framework 3, provide a practical example of how a real-time recommendation system can help therapists better treat their patients in psychotherapy sessions with informative clinical annotations and recommendations of treatment strategies with deep reinforcement learning. Although in this example the strategies are the topics for the therapist to initiate or continue, the same approach can be extended to more complex and nuanced treatment suggestions. For instance, in the ABC approach of cognitive behavioral therapy (CBT), the system (e.g., the system 1000 of FIG. 10) can suggest a belief (B) to guide the patients to better understand the causality between the activating event (A) and its consequence (C).

Another interesting perspective regarding Framework 3 is that while the recommendation agent is driven by reinforcement learning, the therapist (and even the patient) has control over which updates under the reinforcement learning mechanism are used. For instance, the patient can directly offer feedback to the therapists, and given the feedback, the therapist may adjust his or her internal model to weigh on the quality of the suggestions by the recommendation agent.

Next, a particular implementation of Framework 3 will be discussed. The implementation introduces a Reinforcement Learning Psychotherapy AI Companion that generates topic recommendations for therapists based on patient responses. The system uses Deep Reinforcement Learning (DRL) to generate multi-objective policies for four different psychiatric conditions: anxiety, depression, schizophrenia, and suicidal cases (the system can be trained to generate multi-objective policies for other conditions). The proposed virtual psychotherapy AI companion (hereinafter referred to as “AI companion implementation”) provides real-time feedback and recommends treatment strategies to therapists while they are conducting psychotherapy. This implementation can offer interpretable insights by visualizing the policies fine-tuned for different clinical conditions and therapeutic emphases.

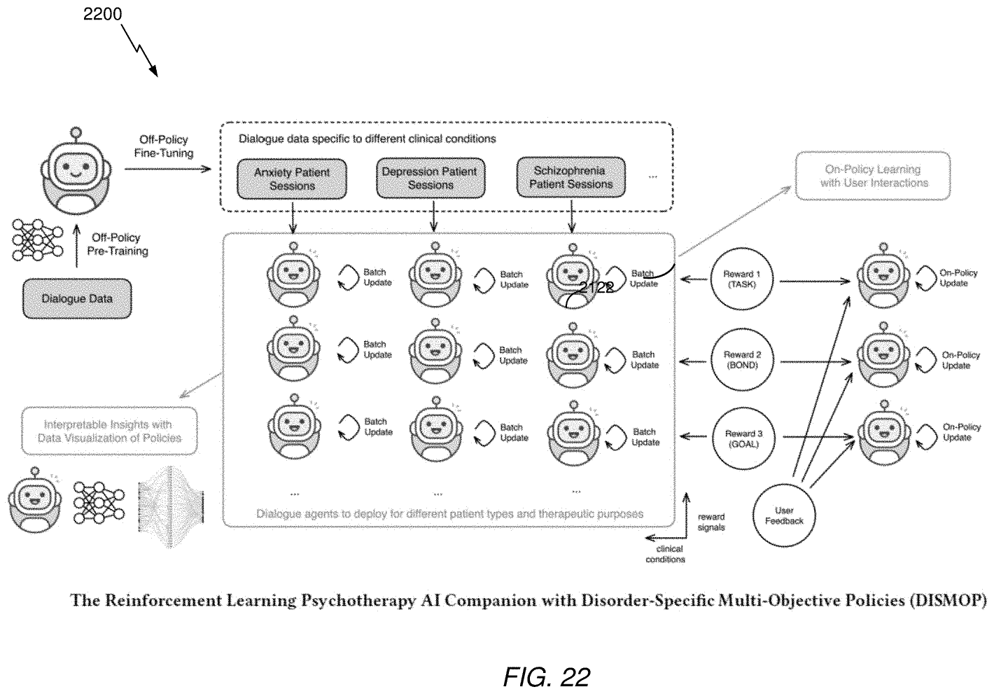

FIG. 22 is a schematic diagram of a reinforcement learning psychotherapy AI companion system 2200 with disorder-specific multi-objective policies (DISMOP). These reinforcement learning agents can be pre-trained in a large text corpus of dialogues and then fine-tuned with off-policy training given historical data of psychotherapy sessions for patients with different clinical conditions. In addition to learning from these historical state transitions, post hoc reward signals can be computed using the computational inference such that each policy has both a disorder specificity and a therapeutic emphasis (e.g., to achieve better bonding between the therapist and the patient). These fine-tuned policies can then be inspected for interpretable insights with policy visualizations and other types of explainable AI approaches or deployed as a psychotherapy AI companion. The autonomous agents can be adaptively updated given certain therapeutic rewards, such as the computational inferred working alliance scores in task, bond, or goal scales, or updated given implicit or explicit user feedback (e.g., the therapist can rate the recommended topics given by the AI companion) or a mixture of reward signals as the on-policy training objectives. The proposed AI companion implementation achieves an interpretable AI framework that can recommend treatment strategies to therapists during psychotherapy sessions, and provides insights into the policies fine-tuned for different clinical conditions and therapeutic emphases, which can help therapists gain a better understanding of the treatment process and improve patient outcomes.

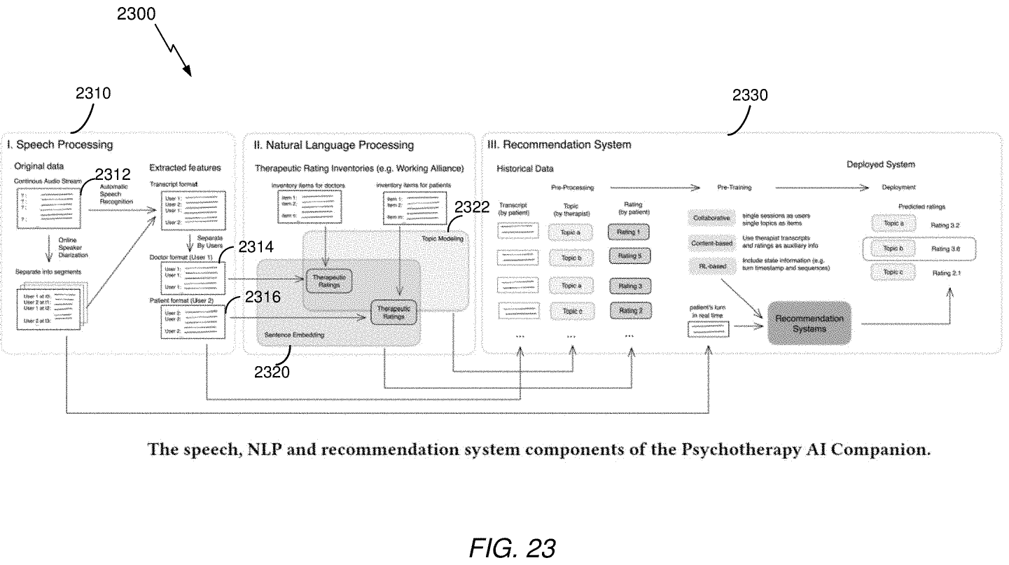

FIG. 23 is a schematic diagram of an analytic system 2300 of the AI companion implementation. The system 2300 includes speaker diarization unit, therapeutic quality rating unit, transcription and real-time rating assessment, topic modeling as recommendation items, recommendation system settings, deep reinforcement learning recommendation approaches, disorder-specific multi-objective policies (DISMOP), and the three levels of the Psychotherapy AI Companion. As shown in FIG. 23, the system 2300 includes a speech processing unit 2310 into which a continuous audio stream 2312 is fed. Speaker diarization using real-time solutions is performed to separate audio into dyads 2314 and 2316 of doctor-patient, which are then transcribed into linguistic turns for downstream analyses.

After obtaining diarization data, the quality assessment setting is configured by specifying a proper inventory. In the example of the system 2300, the Working Alliance Inventory (WAI) is used. Next, both the dialogue turns and WAI items are transformed (by sentence embedding unit 2320) with deep sentence or paragraph embeddings (in this case, Doc2Vec). The cosine similarity between the embedding vectors of the turn and its corresponding inventory vectors are computed to derive a 36-dimension working alliance score for each turn (either by patient or by therapist), which may be saved in a bidirectional relational database (see discussion below regarding example knowledge management systems), or visualized in real-time as a conversation guide.

In the recommendation system 2300, the items the system recommends are treatment strategies, represented as topics that the therapist should initiate or continue for the next turn. Given a large text corpus of many psychotherapy sessions, topic modeling is performed (e.g., by topic modeling engine 2322) to extract the main concepts discussed in the psychotherapy, which can also be directly visualized for interpretable insights. The Embedded Topic Model (ETM), which was shown to create the most diverse concepts in psychological corpus as in this systematic analysis, may be used. Each turn may be annotated with its most likely topic and identified using seven unique topics (see above for list of topics).

As was discussed earlier, the “items” the system 2300 recommends are treatment strategies, which are represented as a topic that the therapist should initiate or continue for the next turn. These “items” are paired with the “users” and “contents,” which, in this case, would be the patientID, their previous turns, their aggregated formats, and other metadata. For instance, it is known that within each session there are many pairs of turns, and that those pairs belong they to the same user. However, one can also assign all turns belonging to one clinical label or all turns related to a certain topic as one “user.” In this example, the session IDs was chosen as the “users.” Lastly, the “ratings” would be the patients' inferred alliance scores predictive of the therapeutic outcomes.

During deployment, the system 2300 registers a session as a new “user” if a session-based item was adopted, and provides punctuated rater evaluations as inference anchors. Next steps include predicting these inference anchors as states, and training chatbots as reinforcement learning agents/engines given these states and neuroscience inspirations.

As noted, reinforcement learning approaches can be effectively applied in language and speech tasks, including recommendation systems. Here three deep RL processes were evaluated: Deep Deterministic Policy Gradients (DDPG), Twin Delayed DDPG (TD3), and Batch Constrained Q-Learning (BCQ).

To further enhance the performance of the recommendation system, the Disorder-Specific Multi-Objective Policies (DISMOP) approach may be used (which on the R2D2 model that was earlier discussed. DISMOP is configured to improve the generalizability of policies across different psychiatric disorders by training on disorder specific datasets. The approach includes a pretraining step and an off-policy batch training process, which uses disorder-specific historical data to learn policies that maximize multiple objectives, such as the therapeutic bond, task agreement, and goal agreement. These policies can then be deployed in suitable settings and incorporate user feedback as an additional reward signal for on-policy updates and real-time improvements.

For the recommendation systems (shown as the unit 2330 of FIG. 23), each session is identified as a user, and the states are frames of dialogues that can be labeled with their topics in real-time, and their ratings with a working alliance (WA) inference module. The reinforcement learning core, powered by deep RL, predicts the best action represented by an embedding for the items (topics). Treating the actions, i.e., predicted topics, as a continuous action space can utilize the innate structure and relationships between the topics, comparing them to a discrete action space. This embedding can be pre-computed, for instance, using dimension reduction techniques to find clusters of different topics in a low-dimensional space. The Doc2Vec embedding of the original dialogue turns was used, averaged by their topic labels, such that each action (i.e., the topic ID) has an averaged representation in the sentence embedding space. This action representation can be translated into a topic label with nearest neighbor, and a given dialogue response will be selected from the historical dialogue data to continue the conversation. The reward can then be computed using the working alliance rate or other types of therapeutic signals, including user feedback from doctors or patients.

The recommendation system 2330 can be extended into three levels. The first level (the backbone) is reinforcement learning-based, which considers the stateful nature of dialogue data. The flexibility of reward signals, i.e., using any rewards, pseudo-rewards, multiple rewards, hybrid rewards, or even inferred rewards, makes policies adaptable to a versatile suite of clinical settings.

The second level is to use additional context, as in content-based recommendation systems. This involves treating the patient turns before the current turns, or all the previous turns up to now, as a feature in the deep reinforcement learning models, by concatenating their sentence embeddings to the states. This provides more context for in-context learning of the generalized models, which can be a foundation model in future work.

In the third level, if given the patient ID and therapist ID, personalized policies with collaborative filtering type recommendation systems can be created, which can potentially improve the compositionality and generalizability of the models for a wide range of populations.

Accordingly, for the implementation of FIG. 23, the procedure 1200 (discussed previously with respect to the more general embodiments depicted in FIGS. 10 and 11) may further include training the recommendation engine according to one or more disorder-specific multi-objective policies using respective disorder-specific training datasets. In such embodiments, the procedure 1200 may further include generating visualization outputs representing interpretable insights for the one or more disorder-specific multi-objective policies the recommendation engine was trained for, the visualization outputs comprising one or more of, for example, topic trajectory plots for the one or more disorder-specific multi-objective policies, or transition matrices for the one or more disorder-specific multi-objective policies.

The performance of the three recommendation agents was evaluated by computing the accuracy of the recommended actions with their corresponding ground truth actions on the test set, with variants of DISMOP being compared (as there were no state-of-the-art or baseline models in this application). Three different scales of working alliance were used for ratings, namely, task, bond, and goal, which measure different aspects of emotional alignments in psychotherapy. Using accuracy to evaluate the recommendation system is a challenging task, as the embedding space can be noisy in the policy generator. Nevertheless, some models using certain therapeutic signals appear to be capturing the real data relatively well. For instance, DISMOP-BCQ-GOAL (with a test accuracy of 0.6424 for all sessions) and DISMOP-DDPG-TASK (with a test accuracy of 0.6406 for anxiety sessions) were the best-performing models, while others provided trivial solutions. For certain disorders, goal scale and task scale appeared to best capture the human therapists' choices, while other ones favored the models trained with bond scores. For instance, DISMOP-DDPG was the recommender winner for anxiety, while DISMOP-TD3 was the winner for depression and schizophrenia, and DISMOP-BCQ was the winner for schizophrenia and suicidal cases. When pooling the sessions of four disorders together, the recommender winner appeared to be DISMOP-BCQ, which may suggest the offline reinforcement learning's advantage in constraining the possible extrapolation errors by the non-offline methods.

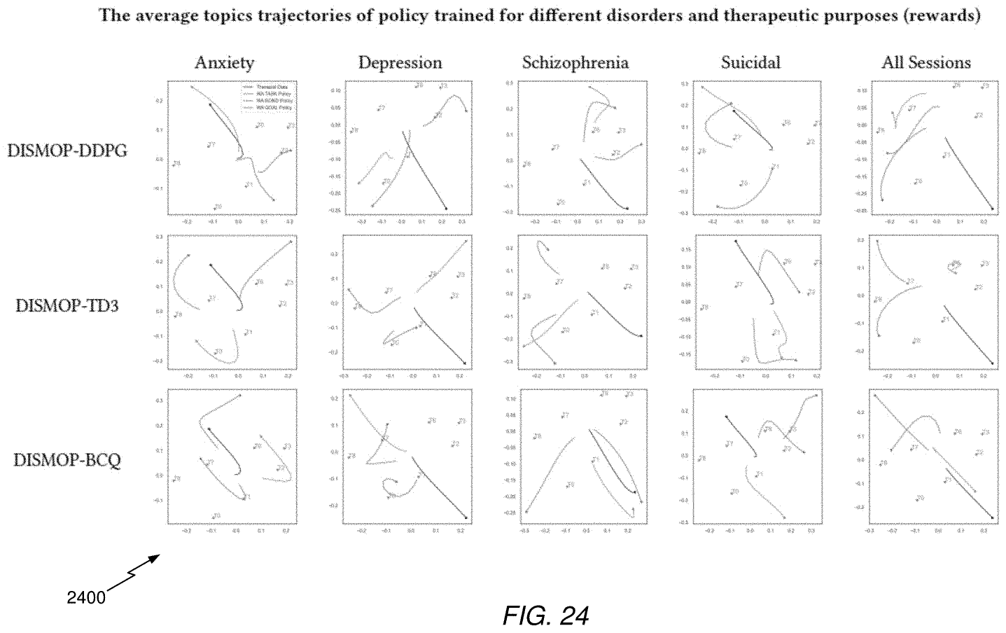

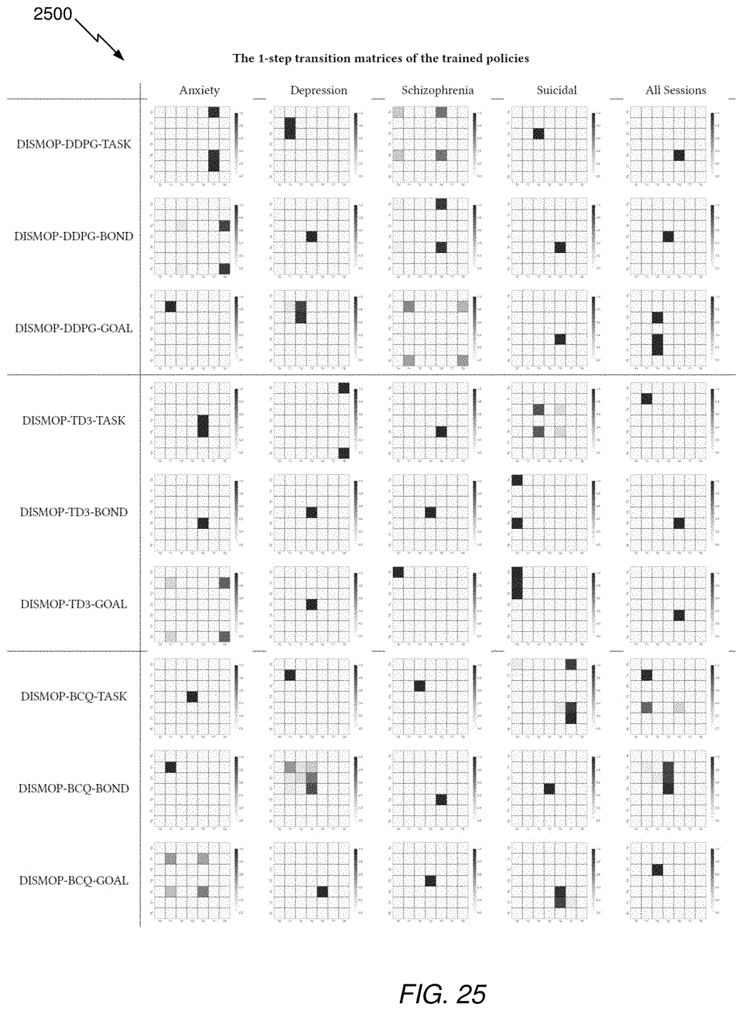

FIG. 24 includes a graph table 2400 presenting the standardized average policy trajectories with respect to the action embeddings (marked with topics) projected onto a 2D principal component analysis space. The trajectories are in the length of 10 past actions (as in the frame size). The end of each trajectory is marked with a larger dot. Distinct patterns of the policies trained with different reward signals were observed, as well as those trained on sessions with different clinical diagnosis. Further analysis can be performed on the policies learned by the DISMOPs by inspecting their transition matrices. FIG. 25 is a graph table 2500 of 1-step transition matrices of the trained policies. As shown in FIG. 25, the 1-step transition matrix normalized by rows reveals interesting patterns in the policies. It was observed that the transition matrices of the DISMOPs mostly converge, with different disorders and therapeutic rewards yielding significantly different matrices. For example, for DISMOP-DDPG trained in depression sessions, if emphasizing the task scale of working alliance, the policy tends to go from talking about sensitive topics like anger, scare, and sadness (topic 2) back to topic 1, which is about play. However, if the policy aims towards the goal scale, it tends to stay focused on discussing anger, scare, and sadness (topic 2). Similarly, for DISMOP-DDPG trained in suicidal sessions, there are recurring discussions about topic 6, which is about explicit ways to deal with stress, such as keeping busy and reaching out for help, which can increase bonding between the doctor and patient and achieve better alliance in their goal scale. However, if the aim is to simply achieve alignment in their tasks during each session, discussing topic 2 is the way to go.

Another interesting example is DISMOP-TD3 trained for schizophrenia patients. It was observed that the best topic to achieve the task scale is to continuously discuss topic 6 (dealing with stress), but if the aim is to achieve the bond scale, the focus should be on topic 3 (anger and sadness). If the goal scale is targeted, the policy tends to focus on topic 0 (figuring out and self-discovery).

These insights provide a deeper understanding of the learned policies and how they can be interpreted in the context of psychotherapy. The visualization and interpretation of the DISMOPs' policy dynamics offer valuable insights into the underlying decision-making processes and can help in understanding how the policies are shaped by different disorders and therapeutic rewards. Overall, these visualization analytics demonstrate that the policies learned by different reinforcement learning agents are distinct and reveal patterns that are consistent with what is known about their underlying therapeutic signals.

Thus, the AI companion implementations described herein was shown to be an effective recommendation platform, especially when combined with reinforcement learning, and also provides insights into how different reward signals affect the recommendation policies learned by the system. In addition, interpretable insights into the recommendation policies was determined through the use of visualizations, such as trajectory and transition matrix plots.

The implementation of the system depicted in FIG. 23 can be improved in several ways. First, more advanced reinforcement learning algorithms/processes, such as actor-critic methods or proximal policy optimization, may be used. Additionally, more sophisticated embeddings, such as contextual embeddings or knowledge graph embeddings, can be used to further improve the quality of the recommendations. In addition, use of user feedback to refine the recommendations and personalize them for individual patients may also improve the performance of the system.

The testing and evaluation of the system 2300 of FIG. 23 showed that the use of DISMOP provides interpretable insights into the policies learned by the system. Further improvements to the performance of the system may be achieved through other interpretability techniques, such as attention mechanisms or saliency maps. These approaches could help to shed further light on the inner workings of deep reinforcement learning models and provide insights into how they can be improved.

The DISMOP approach can also be extended to incorporate interpretable policies that enable more transparent and ethical decision-making. For example, by visualizing the learned policies and analyzing the transition matrices of the DISMOPs, insights are gained into the decision-making process of the AI companion, and thus more safeguards are provided against potential bias and stereotypes. These insights can provide valuable information to clinicians and researchers for improving the quality of care and advancing the field of psychotherapy in a responsible and safe way. The proposed approaches and solutions can also be extended to incorporate natural language generation capabilities to enable the AI companion to generate responses to patients in real-time, providing more timely and personalized care. Finally, further improvement can be achieved through the integration of other types of data, such as physiological signals and behavioral data, to improve the accuracy and effectiveness of the recommendation systems.

It is also to be noted that the technology described herein could be used with state-of-the-art foundation models, such as Generative Pre-Trained Transformers (GPT), the base for ChatGPT etc. Below is a proposed procedure for training a foundation model, say, using GPT-3, for Psychotherapy applications.
1) Data collection: The first step is to collect data that will be used to train the model. In the case of psychotherapy applications, this data may include transcripts of psychotherapy sessions, notes taken by therapists, and even social media posts related to mental health.
2) Data cleaning and preprocessing: Once the data has been collected, it may be cleaned and preprocessed to ensure that it is usable for training the model. This may involve removing irrelevant or sensitive information, such as identifying information about patients, and standardizing the data format.
3) Training the model: The cleaned and preprocessed data is then used to train the foundation model, such as GPT-3, using a process known as supervised learning. During this process, the model is exposed to examples of input and output pairs, such as a patient's statement and a corresponding therapeutic response. The model learns to recognize patterns in the data and generate appropriate responses based on those patterns. They can also be trained using self-supervised learning (SSL) methods, or reinforcement learning with human feedbacks (RLHF) if human annotators are available to score the quality of the outputs.
4) Fine-tuning the model: After the model has been trained on the initial data, it may be necessary to fine-tune the model for specific psychotherapy applications. For example, the model may be further trained on data related to a specific disorder, such as depression or anxiety, to improve its ability to generate relevant responses for that disorder (one way could be the above DISMOP framework).
5) Evaluation and testing: Finally, the model must be evaluated and tested to ensure that it is generating appropriate responses for the intended application. This may involve testing the model on new data that it has not seen before or conducting user studies to gather feedback from therapists and patients.

The goal of training a foundation model for psychotherapy applications is to teach the model to recognize patterns in the data and generate appropriate responses to support the therapeutic process. Properly learning from the data involves careful cleaning and preprocessing, effective training and fine-tuning, and rigorous evaluation and testing. The application tasks may include generating therapeutic responses, identifying potential mental health concerns, or providing personalized treatment recommendations based on a patient's history and symptoms.

### Framework 4: Speaker Diarization with Reinforcement Learning

Speaker diarization is the task of labelling an audio or video recordings with the identity of the speaker at each given time stamp. In each time window, speaker recognition is performed to distinguish the identity of the person who is speaking in a mixed-speaker signal based on voice characteristics. Conventional diarization approaches include two principal steps: registration and identification. The registration step computes a voiceprint model of each speaker given his or her acoustic samples, while the identification step matches existing voiceprint model with real-time audio signal. However, in real life, requiring all users to complete voiceprint registration prior to, for example, a multi-speaker teleconference may be impractical.

The implementations discussed herein for Framework 4 are configured to perform real-time multi-speaker diarization and recognition without prior registration or pretraining. The proposed framework is based on a fully online implementation using reinforcement learning setting. Various reinforcement learning solutions, and their respective practical considerations, are discussed. The proposed approaches and solutions pertaining to Framework 4 may be combined with strategies such as learning from historical data using offline reinforcement learning, dealing with sparse feedback with semi-supervision, and boosting transfer learning with domain adaptation. The proposed diarization system implementations discussed herein may be used in conjunction with any of the frameworks of the present disclosure to perform any of the diarization operations required by those other frameworks.

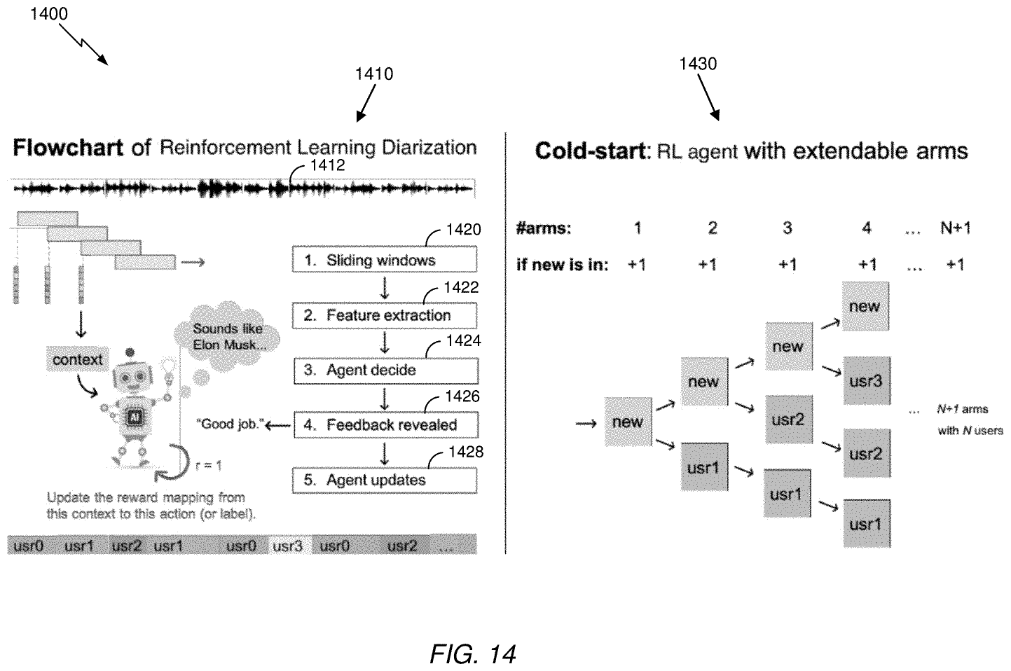

FIG. 14 is a diagram of the reinforcement learning diarization system 1400. The left hand-side 1410 of FIG. 14 provides a flowchart of the reinforcement learning-based speaker diarization system, and the right-hand side 1430 illustrates a speaker expansion process of the reinforcement learning system whereby the diarization engine can be continually reconfigured to adaptively learn to identify new users as the system is exposed to additional speech segments associated with new speakers. The implementations of the proposed approaches and solution discussed herein allow the framework to learn continually by learning to detect speaker identity on the fly through reward feedbacks, and to be operational without any pretraining preceding deployment. As previously mentioned, a preferable artificial intelligence (AI) engine for such a realistic speaker recognition and diarization system should (1) not require user registrations before its deployment, (2) allow new user to be registered into the system in real-time, (3) transfer voiceprint information from old users to new ones, and (4) be up and running without pretraining on large amount of data in advance. Requirement 4, as previously noted, introduces an additional caveat that the labeling of the user profiles happens on the fly, trading off models pretrained on big data with the user directly interacting with the system by correcting the agent as labels.

The proposed framework 4 illustrated in FIG. 14 implements, in some embodiments, the following process. A segment 1412 (usually obtained by a sliding window 1420 process) of speech information is parsed and fed into a feature extractor 1422, which can be implemented as a neural network model or other analytic/algorithmic feature extraction engine (such as a Mel Frequency Cepstral Coefficient (MFCC) filtering engine). The diarization system 1400 (also referred to as a diarization agent) takes the extracted features as the input, and adaptively decides on an action, which in the present example is the determination of a speaker ID the agent believes that is currently speaking. If a user/administrator managing the system is around and would like to offer a feedback (e.g., indicating, via an interface represented as “Feedback revealed” box 1426, whether the diarization system made a correct determination), then human annotations are provided as rewards. The feedback can be a correcting term on the user side, which would likely be sparse, but can nevertheless facilitate the reinforcement learning to update its policy accordingly, such that in the future the diarization system will make better decisions. The feedback is used to cause updates, at box 1428, to the machine learning diarization parameters (defining the machine learning behavior of the system). In some embodiments, the system is configured to operate according to an interactive learning model with cold-start arms and episodically revealed rewards (users can either reveal no feedback, approving by not intervening, or correcting it). This cold-start arms approach is illustrated by the diagram panel 1450 of FIG. 14, showing the arm expansion process of reinforcement learning agents for cold-start problems.

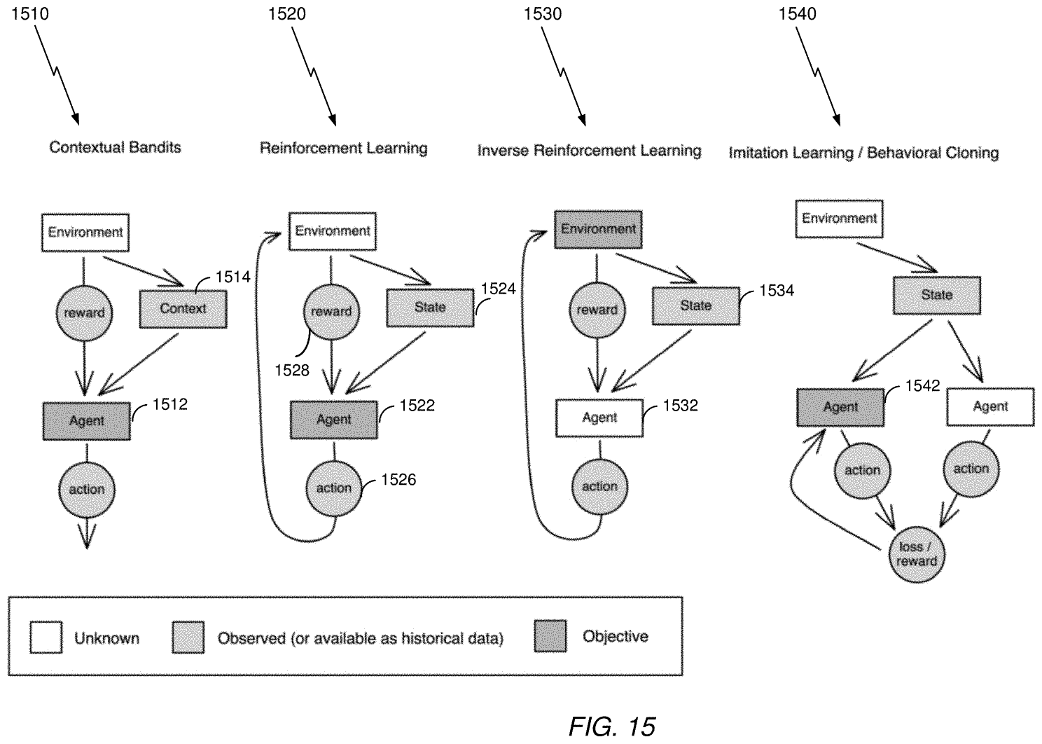

There are several classes/types of reinforcement learning approaches that may be used in conjunction with the framework proposed herein. FIG. 15 includes diagrams illustrating operations of four different classes of reinforcement learning processes that may be used in conjunction with the diarization system implementations of Framework 4.

A first reinforcement learning class is the contextual bandits reinforcement learning class illustrated by diagram 1510 of FIG. 15. At each time point (iteration) t∈{1, . . . , T}, an agent 1512 is presented with a context 1514 (feature vector) xₜ∈R^{N} before choosing an arm k∈A={1, . . . , K}. X={X₁, . . . , X_{N}} denotes the set of features (variables) defining the context. Let rₜ=(r¹ₜ, . . . , r^{K}ₜ) denote a reward vector, where r^{k}ₜ∈[0, 1] is a reward at time t associated with the arm k∈A. Herein, the focus will be primarily on the Bernoulli bandit with binary reward, i.e., r^{k}ₜ∈{0, 1}. Let π: X→A denote a policy. Also, D_{c,r} denotes a joint distribution over (x, r). Assume that the expected reward is a linear function of the context, i.e., E[r^{k}ₜ|xₜ]=μ^{T}ₖxₜ, where μₖ is an unknown weight vector associated with the arm k.

Another class of reinforcement learning is the Markov decision processes (MDP) class of processes/algorithms for solving problems modeled as MDP. An MDP is defined by the tuple (S,A, T ,R, γ), where S is a set of possible states (at a box 1524), A is a set of actions (occurring at node 1526), Tis a transition function defined as T(s, a, s′)=Pr(s′|s, a), where s, s′∈S and a∈A, and R: S×A×S→R is a reward function (evaluated at node 1528), γ is a discount factor that decreases the impact of the past reward on current action choices. Typically, the objective is to maximize the discounted long-term reward, assuming an infinite-horizon decision process, i.e., to find a policy function π: S→A which specifies the action to take in a given state, so that the cumulative reward is maximized according to, max_{π}Σₜ₌₀^{∞}γ^{t}R(sₜ, aₜ, sₜ₊₁).

Inverse reinforcement learning, illustrated by diagram 1530, is another class of reinforcement learning that first tries to learn the underlying rewards and then use this learned rewards as a training environment to further train the reinforcement learning agents (an example of which is illustrated as node 1532).

A fourth class of reinforcement learning processes is the imitation learning and behavioral cloning processes, illustrated by diagram 1540. There are several approaches for imitation learning. One imitation learning is the Behavior Cloning with Demonstration Rewards (BCDR), which is a novel training procedure and agent for solving this problem. In this setting, an agent 1542 first goes through a constraint learning phase where it is allowed to query the actions and receive feedback r^{e}ₖ(t)∈[0, 1] about whether or not the chosen decision matches the teacher's action (from demonstration). During the deployment (testing) phase, the goal of the agent is to maximize both rₖ(t)∈[0, 1], the reward of the action k at time t, and the (unobserved) r^{e}ₖ(t)∈[0, 1], which models whether or not the taking action k matches which action the teacher would have taken. During the deployment phase, the agent receives no feedback on the value of r^{e}ₖ(t), where it would be desirable to observe the behavior captures the teacher's policy profile. In the specific problem at hand (diarization using reinforcement learning), the human data plays the role of the teacher, and the behavioral cloning aims to train the agents to mimic the human behaviors.

Further examples of reinforcement learning (RL) processes/algorithms include processes that are based on the deterministic policy gradient in an actor-critic architecture, such as the Deep Deterministic Policy Gradients (DDPG) process that is a model-free procedure for continuous action spaces, and has been shown to successfully learn policies end-to-end. Another possible RL process that may be used is the Twin Delayed DDPG (TD3) that builds on a Double Q-Learning approach, and provides a solution to correct for an overestimated value issue to yield more competitive results in various game settings.

There are several strategies that may be used in conjunction with the implementation of a reinforcement learning approach. One such strategy is to use deep-learning based reinforcement learning. Another strategy that may be employed is that of batched and offline reinforcement learning. The offline reinforcement learning learns from historical data of behavioral trajectories. Since online data collection of RL models are usually time consuming, in real world industrial settings these models are sometimes trained using previously collected data. As a result, there is a growing popularity of offline reinforcement learning approaches. Among those approaches is the Batch Constrained Q-Learning (BCQ) approach that implements a continuous control deep RL algorithm that yields competitive results in off policy evaluations by restricting the agent's exploration in the action space. In the context of reinforcement learning for diarization systems, a history of previous speaker diarization sessions can be used to better improve the performance of reinforcement learning training. A popular method may include Conservative Q-Learning. A further implementation strategy is that of transfer learning. In many cases it is desirable to use what has been learned from previous successful diarization tasks. For instance, the speaker diarization system trained on adult speech corpus can be helpful to kick start a system for kids.

The reinforcement learning-based diarization systems described herein are adaptive, lightweight, and generalizable to new users. Such systems do not have to register the users beforehand, and they do not have to know how many users will be joining this conversations. These types of diarization systems are useful for multi-user teleconferences, where many people might come and go, generally without performing pre-registration ahead of time.

Thus, in some variations, a diarization system is provided that includes a receiver module to obtain a speech segment, and a processor-based controller, coupled to one or more memory devices. The controller is configured to extract one or more speech features from the speech segment, process the one or more extracted speech features with a configurable machine learning diarization engine adapted to identify a speaker associated with the speech segment, and adjust weights of the configurable machine learning diarization engine according to one or more reinforcement learning approaches in response to receipt of feedback indicative of accuracy of the speaker identified by the diarization engine to a true speaker identity for the speech segment. In some additional variations, a non-transitory computer readable media is provided that includes computer instructions executable on a processor-based device to extract one or more speech features from the speech segment, process the one or more extracted speech features with a configurable machine learning diarization engine adapted to identify a speaker associated with the speech segment, and adjust weights of the configurable machine learning diarization engine according to one or more reinforcement learning approaches in response to receipt of feedback indicative of accuracy of the speaker identified by the diarization engine to a true speaker identity for the speech segment.

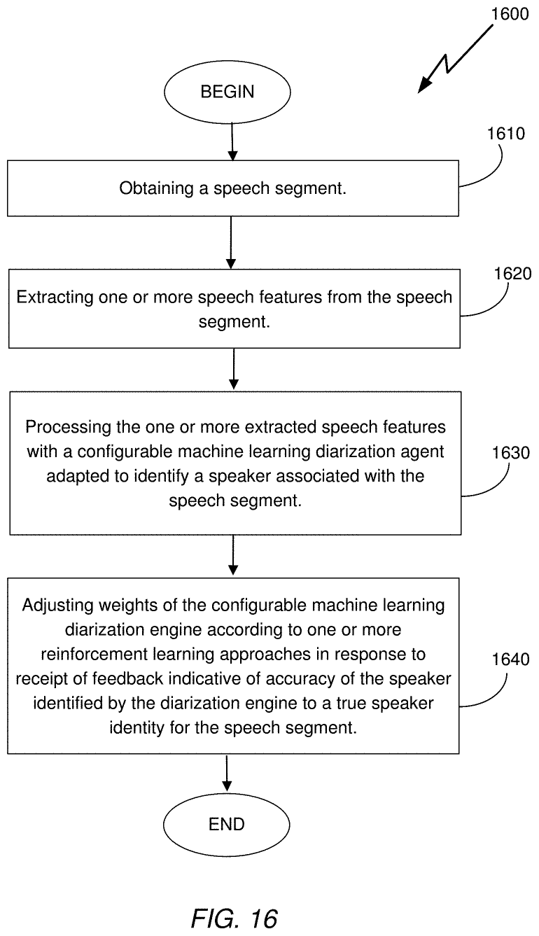

With reference to FIG. 16, a flowchart of an example procedure 1600 for multi-speaker diarization is shown. The procedure 1600 includes obtaining 1610 a speech segment, extracting 1620 one or more speech features from the speech segment, processing 1630 the one or more extracted speech features with a configurable machine learning diarization engine adapted to identify a speaker associated with the speech segment, and adjusting 1640 weights of the configurable machine learning diarization engine according to one or more reinforcement learning approaches in response to receipt of feedback indicative of accuracy of the speaker identified by the diarization engine to a true speaker identity for the speech segment.

In some embodiments, adjusting the weights of the configurable machine learning diarization engine may include adjusting the weights of the configurable machine learning diarization engine according to one or more reinforcement learning approaches that include, for example, a model-based reinforcement learning approach, a model-free reinforcement learning approach, an inverse reinforcement learning approach, or an imitation learning and behavioral cloning approach. In such embodiments, any of the one or more reinforcement learning approaches is implemented according to one or more of, for example, a deep learning process, a transfer learning process, a semi-supervised learning process, and/or a self-supervised learning as auxiliary model components process.

In various examples, adjusting the weights of the configurable machine learning diarization engine may further include determining that the speaker associated with the speech segment is a new speaker not previously associated with previous speech segments processed by the machine learning diarization engine and configuring the machine learning diarization engine to generate a new label, associated with the new speaker, in response to processing subsequently obtained speech segments associated with the new speaker.

Adjusting the weights of the configurable machine learning diarization engine further may include one or more of, for example, performing deep-learning-based reinforcement learning to adjust the weights of the configurable machine learning diarization engine, performing batched and offline reinforcement learning to adjust the weights of the configurable machine learning diarization engine, and/or performing transfer learning process to adjust the weights of the configurable machine learning diarization engine based on existing weights of one or more other trained configurable machine learning diarization engines.

### Framework 5: Knowledge Management with Relational Databases

Knowledge management systems are in high demand for industrial researchers, chemical and/or research enterprises, or evidence-based decision making. However, existing systems have limitations in categorizing and organizing paper insights or relationships. Traditional databases are usually disjoint with logging systems, which limit its utility in generating concise, collated overviews. Consider the application of reference management of academic researchers as an example. Knowledge management systems are often used by researchers to keep track of papers or subsets of papers. Usually, the research information of different papers or references has meta information that can be filtered and sorted. An example scenario would be: a scientist logs or inputs a particular paper into a system, with each entry containing many meta information about the papers. These meta information elements can be filtered or sorted (e.g., by year, journal, author, etc.) Each paper might contain multiple concepts or topics, and each topic might be germane to multiple papers.

Disclosed are implementations (including hardware, software, and hybrid hardware/software implementations) directed to a knowledge management system that utilizes relational databases to log hierarchical information with connected concepts. This knowledge management framework (referred to as Framework 5) can be used to facilitate research and writing processes, or generate useful knowledge from references or insights from connected concepts. This knowledge management framework enables novel functionalities encompassing improved hierarchical notetaking, AI-assisted brainstorming, and multi-directional relationships. Potential applications include managing inventories and changes for manufacture or research enterprises, or generating analytic reports with evidence-based decision making.

The present framework can also be used to collect and organize information procured during psychotherapy sessions in order to dynamically adapt the performance of machine learning models used to analyze psychotherapy data, to implement reinforcement procedures to improve performance of the various psychotherapy analysis frameworks (such as those described herein in relation to Frameworks 1-4 and 6), and to implement recommendation systems (e.g., to aid and train therapists by providing treatment strategies). Thus, the knowledge management framework described herein can be deployed in implementations of the technologies of Frameworks 1-4 and 6 for joint use in therapy settings, as observational insights collected via the analytical engines of the Frameworks 1-4 and 6 can be stored in separate relational databases, and accessed by an interventional engine, which can store suggestions and other insights into upstream relational databases for real-time visualization.

A specific example application is a knowledge management system for academic papers/references (although the proposed system can be used to process and manage other types of documents). A scientist logs/inputs a particular paper into a system, with each entry containing many meta information about the papers. These meta information elements can be filtered/sorted (e.g., by year, journal, author, etc.). Each paper might contain multiple concepts or topics, and each topic might be associated with multiple papers. In some cases, the system can automatically assign topics to some papers based on text data mining. The user can filter the papers by topics. Within each paper, during the reading, the scientist might want to log an insight or note on certain paragraphs. Sometimes the notes can be about multiple papers, and their relationship can be in various types. These notes or insights also have topic tags, which can optionally be automatically curated. The system can also generate useful concepts or knowledges as well as their references to facilitate the research and writing process of the scientist.

The proposed framework can thus be used as a management system, a knowledge generating system, and/or a companion for evidence-based decision making system. Possible commercial applications in which the proposed framework can be used include:
1. Products and services for academic or industrial researchers. The proposed framework enables novel functionalities that encompasses notetaking (e.g., hierarchical notetaking), AI-assisted writing or brainstorming, determining multi-directional relationships, and other types of analyses.
2. Managing inventories and changes for manufacture, chemistry, or research enterprises. The inventories or measurements of factories usually involve dependency and hierarchical interactions. Traditional databases are usually disjointed with separate logging systems. The proposed framework can enable useful curation function to offer useful and concise reporting regarding key events or phenomena (like the topics). These can provide important insights for promoting safety (e.g., in factories). The proposed framework can also be used as an organizational tool for industries with high-volume data, and as a way to conduct internal auditing tool for employee metrics.
3. Evidence-based decision making. In big companies, critical decisions are usually made with a group of market researchers or consulting firms that come up with various analytic reports. The proposed framework can provide a fast and evidence-based solution by generating a report (given the keyword or topic as input) collating the hierarchical and intra-connected records. This hierarchical knowledge graph can serve as a useful primer in important decision making processes and guide the investigators to locate relevant resources.
4. As noted, Framework 5 may also be used to maintain medical notes and data regarding patients. For example, the knowledge management system can be used to store transcripts from psychotherapy sessions, and include automatically generated notes about treatment strategies, insights, psychotherapy analysis and inferences, that are produced through various machine learning engines (that infer insight and topic labels based on the semantic meaning of transcript segments). The storage of such psychotherapy transcript data can be performed per segments of the transcripts (e.g., per speech turn by different individuals partaking in the sessions).

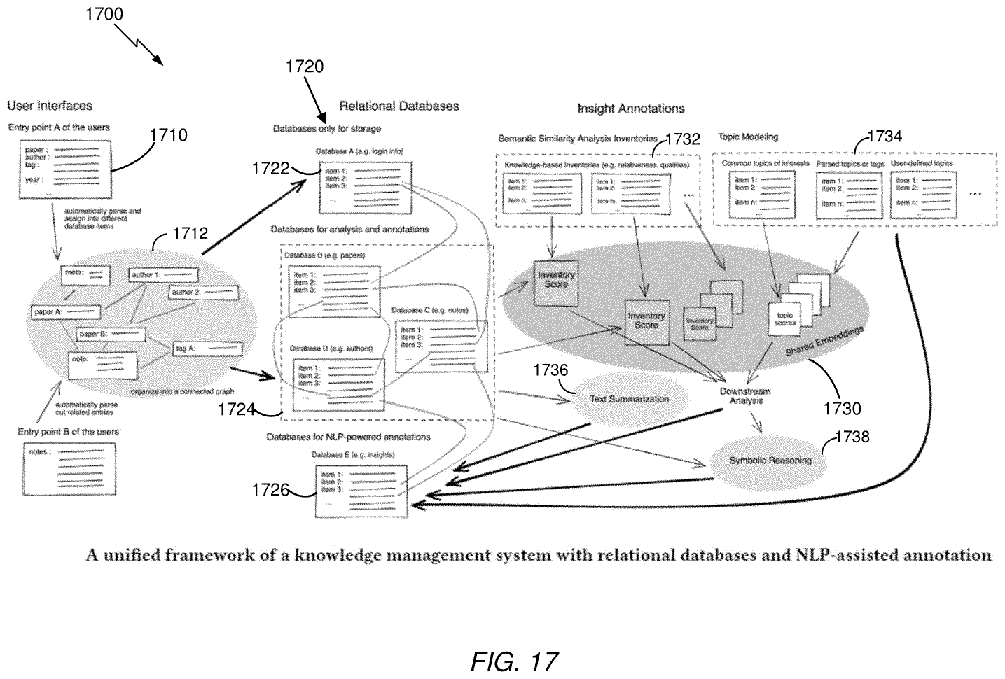

Thus, with reference to FIG. 17, a schematic diagram of an example knowledge management system 1700 with relational databases and insight annotation powered by natural language processing (NLP) is shown. A user interface 1710 provides the entry points into the knowledge management system 1700. Different interfaces introduce different routes, but they generally involve a parsing and extraction processes (collectively schematically represented as an ellipse 1712) to atomize the user inputs into nodes that connects in a small knowledge graph. This graph is then placed into a relational database 1720 where its links are preserved. The relational databases may include three parts. Some databases in the relational databases are only used for storage (such as database A, marked as database 1722). Some of the database (e.g., databases 1724, comprising, in the example illustration of FIG. 17, databases B, C, and D) are used for analysis and annotations. Additional databases, such as database 1726 (identified as database E), are kept to store annotated insights or other downstream analytical artifacts.

As further shown in FIG. 17, there are several routes that can utilize natural language processing (through algorithmic processes or machine-learning processes, including machine learning processes based on language transform models) to generate and annotate insights within databases. In principle, any sentence or paragraph embedding can help characterize a document and inventories of interest. For instance, the Doc2Vec embedding is a popular unsupervised learning model that produces vector representations of sentences and text documents. The Doc2Vec transform improves upon the traditional bag-of-words representations by utilizing a distributed memory that remembers what is missing from the current context. SentenceBERT is another popular option which modifies a pretrained BERT network by using siamese and triplet network structures to infer semantically meaningful sentence embeddings. Other embedding types can also be used. With these deep embeddings, document entries from the relational databases or input from the user interface are transformed (embedded) into vectors by one or more transform and analysis unit (schematically represented as an ellipse 1730). Subsequently, the cosine similarity between vectors at certain turn can be compared with inventory entries (by semantic similarity analysis inventories unit 1732, which may be part of the transform and analysis unit 1730). With that, for each text segment, an N-dimension score is derived for the property. For instance, the inventory can be a written guidelines that evaluate the usefulness of certain documents, say, a list of leadership principles that some companies use to evaluate a candidate's resume, work report, or performance review form. And the relational database could be hosting an employee's self-reported performance review form. The system can automatically compute a score based on each item of the guidelines and annotate these document entry accordingly. Other applications can evaluate the patient-doctor alignment from an automatically transcribed psychotherapy sessions based on a clinical questionnaire inventory, etc.

The proposed framework is also used, in preferred embodiments, for topic modeling and classification. In natural language processing and machine learning, a topic model is a type of statistical graphical model that helps uncover the abstract “topics” that appear in a collection of documents. The topic modeling technique (implemented by topic modeling unit 1734, which may be part of the transform and analysis unit 1730) is frequently used in text-mining pipelines to unravel the hidden semantic structures of a text body. This can be very handy in annotating the database entry. For instance, a user scenario could be in a consumer-facing chatbot, where the dialogue between the client and agent is transcribed, and a topic modeling analysis is automatically performed to generate a list of discussed topics and their scores based on semantic similarity. Several neural topic models include the Neural Variational Document Model (NVDM) (an unsupervised text modeling approach based on variational auto-encoder), Gaussian softmax construction (GSM) (a NVDM variant), the Wasserstein-based Topic Model (WTM), the Embedded Topic Model (ETM), and other models.

Another feature of the framework proposed herein is text summarization (implemented by text summarization unit 1736). When the scales of the databases used increase, maintaining the interpretability of the knowledge management system becomes more challenging. The expanding availability of documents and entries inside the database cannot yield actionable insights without proper aggregation. The field of automatic text summarization deals with this problem by producing a concise and fluent summary while preserving key information content and overall meaning. For instance, the database entries (such as paper abstracts, or reading notes as in the reference manager example) can first be grouped or clustered by their semantic similarity or inferred topics. Within each group, condensed descriptions are generated. A user case could include automatically generating writing outlines or topics based on the available references and reading notes in a paper reference manager. In the active field of text summarization, extraction and abstraction are the two main approaches. The extractive summarization techniques generate summaries by choosing a subset of the sentences in the original text, by computing first an intermediate representation of the text, deriving a sentence score, and finally performing a subset selection operation onto the original texts. The abstraction approach uses latent semantic analysis, frequency-driven approaches, and topics modeling.

A further feature of the framework proposed herein is the symbolic reasoning feature (performed by symbolic reasoning unit 1738). While topic modeling offers interpretable subjects, and text summarization offers interpretable paragraphs, the logic and causal relationship between these insights can be arbitrary. The field of symbolic AI bridges this gap by introducing high-level and human-readable symbolic representations into these practical problems. Such processing can potentially derive logic programming rules and semantic relationships that can be use as actionable knowledge graphs. Recently, there has also been increasing interest in a modern approach called neuro-symbolic AI, where the well-founded knowledge representation and reasoning from the symbolic perspective are integrated with deep learning from a statistical perspective. This offers both effective predictive power and necessary explainability for many real-world applications.

When designing an interconnected and intelligent knowledge management systems for a domain-specific application, there are some practical questions to be considered:
Database consideration: What are the storage capacities of this technology?
User interface: What visual and user interface is preferred by users?
Organizational benefits: What specific organizational functionality would this system provide over current systems?
Latency and responsiveness: What are the synchronization capacities of this technology across devices?
Customization: Can users modify or customize this system to their own preferences?
Security: Would this technology allow for secure encryption or storage of higher value data?
Collaboration: Would this system allow for collaborative use by multiple stakeholders?
Investigation: What kind of insights or investigations would be desirable to gain from this system?
I/O: Would this system allow import or export from other knowledge management systems?

Other than these practical questions to consider, a more thorough design process would involve market analysis (market size, emerging technologies, policies, challenges, new trends, and policies), domain analysis (systematic activity for deriving, storing domain knowledge to support the engineering design process as in), business process modeling (i.e., identifying the lead processes and subprocess of outgoing products) and architecture design with viewpoints (stakeholder concerns, context diagram, decomposition view, uses view, and deployment view). Sometimes, case studies are also useful.

Thus, in summary, Framework 5 proposes solutions and approaches to address the applied problem of a knowledge management systems that host information that contain multiple and bi-directional relationships in layers of metadata, the application domains, user scenarios and the existing approaches in the fields, and constructs a framework for a knowledge management system with relational database and NLP-assisted insight annotation. The framework comprises a knowledge management system that includes a user interface to provide input and present output relating to one or more documents or sensors. The framework maintains a relational database storing information relating to the one or more documents, and executes knowledge parsing and extraction processes (e.g., implemented on a parsing and extraction unit) in communication to the user interface and the server. The framework can determine at a first time instance the metadata information elements associated with the particular document entry. The databases can then be automatically annotated with NLP techniques such as semantic similarity analysis, topic modeling, text summarization and symbolic reasoning. A knowledge graph can then be learned from these language models to be used as interpretable insights for real-world downstream tasks.

Accordingly, in various example embodiments, a knowledge management system is provided that includes a user interface to provide input and present output relating to one or more documents, one or more memory devices to maintain a relational database storing information relating to the one or more documents, and a processor-based controller, in communication with the user interface and the one or more memory devices. The controller is configured, for a particular document, to determine at a first time instance metadata information elements associated with the particular document, and include in a particular record of the relational database associated with the particular document at least some of the metadata information elements determined at the first time instance in one or more of a plurality of fields of the particular record. The plurality of fields includes at least, for example, a) a document-specific concepts field to maintain concepts specific to the particular document, and b) common concepts field to maintain common concepts shared by a plurality of documents associated with a plurality of records in the relational database. The controller is further configured to include in the particular record of the relational database, at one or more subsequent time instances, one or more document-specific user notes for storage in a document-specific notes field, and one or more general documents user notes, determined by a machine learning engine analyzing other records in the relational database, for storage in a common notes field of multiple records of the relational database sharing the general documents user notes. The particular document may include one of, for example, a scholarly article written by a user, or user records for the user.

In various embodiments, the one or more documents may include transcripts generated for psychotherapy sessions. In some examples, the processor-based controller configured to determine the metadata information elements may be configured to divide the particular document into one or more semantic segments, and apply one or more machine learning processes to the one or more semantic segments to derive annotation data for the particular document. In such examples, the processor-based controller configured to apply the one or more machine learning processes to derive annotation data for the particular document may be configured to perform topic modeling analysis on one or more of, for example, the one or more semantic segments of the particular document, or segments of other documents associated with other records of the relational database. The processor-based controller configured to apply the one or more machine learning processes to derive annotation data for the particular document may be configured to determine, using a vector-transformation-based machine learning engine, semantic similarity between the one or more segments of the particular document and one or more semantic items in at least one inventory of topics and concepts. In additional examples, the processor-based controller configured to apply the one or more machine learning processes to derive annotation data for the particular document may be configured to generate semantic summarization for the particular document based on one or more of, for example, an extractive summarization techniques, or latent semantic analysis technique. Also, the processor-based controller configured to apply the one or more machine learning processes to derive annotation data for the particular document may be configured to perform a symbolic reasoning analysis on the one or more segments of the particular document to determine logical and causal relationship between concepts associated with the semantic content of the one or more segments for the particular document. In another example embodiment, the processor-based controller may further be configured to determine, using a machine learning process, at least one of the common concepts shared by the plurality of documents based on semantic similarity between the concepts specific to the particular document and respective document-specific concepts for at least some of the plurality of documents.

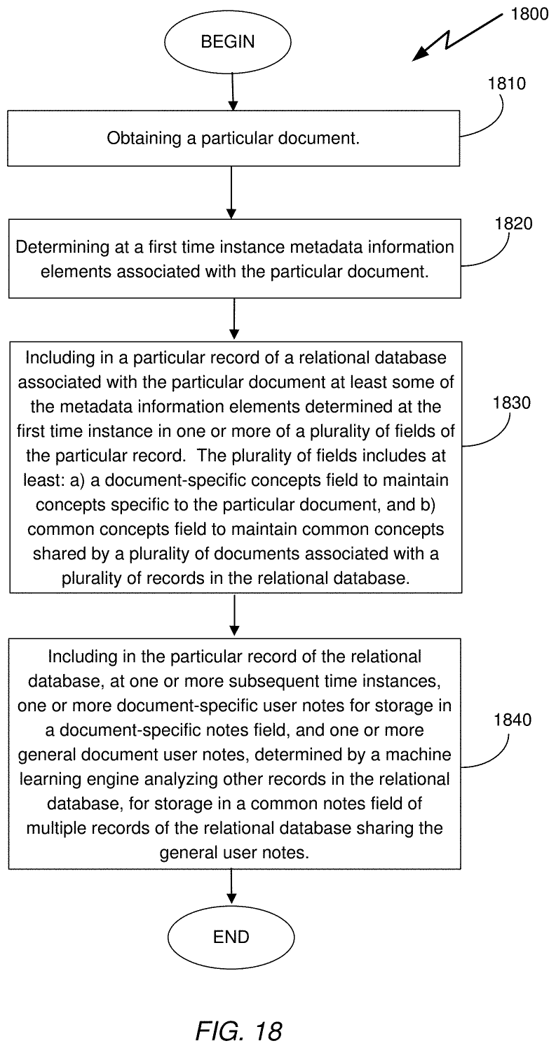

With reference to FIG. 18, a flowchart of an example procedure 1800 for knowledge management processing is shown. The procedure 1800 includes obtaining 1810 a particular document, determining 1820 at a first time instance metadata information elements associated with the particular document, and including 1830 in a particular record of a relational database associated with the particular document at least some of the metadata information elements determined at the first time instance in one or more of a plurality of fields of the particular record. The plurality of fields includes at least: a) a document-specific concepts field to maintain concepts specific to the particular document, and b) common concepts field to maintain common concepts shared by a plurality of documents associated with a plurality of records in the relational database. The procedure 1800 additionally comprises including 1840 in the particular record of the relational database, at one or more subsequent time instances, one or more document-specific user notes for storage in a document-specific notes field, and one or more general document user notes, determined by a machine learning engine analyzing other records in the relational database, for storage in a common notes field of multiple records of the relational database sharing the general user notes. Embodiments of the procedure 1800 include any of the features described herein, including any one or more of the features discussed in relation to the knowledge management system.

### Framework 6: Visualizing Therapy Sessions with Temporal Topic Modeling and AI-Generated Arts

Framework 6 builds on the approaches and solutions developed for Framework 1, in which dialogue data (transcript of a psychotherapy session involving one or more patients and one or more therapists) is analyzed using a machine learning psychotherapy model. The analysis performed by the ML engine produces weighted topic labels representative of the semantic content of dialog segments (arranged in a temporal series of topic labels). In the present Framework 6, data derived from the transcript data and/or the topic labels output is used to generate image data that provides a visual representation of a patient's psychotherapy data, and thus can provide a rolling temporal visual representation of emotional/psychological progress of the psychotherapy session. This visual representation can give the therapist, for example, visual cues of the mood of the patient and the effectiveness of the psychotherapy session, and consequently allow the therapist to quickly respond in a therapeutically appropriate manner (e.g., to adjust the direction of the session and modulate the course of treatment of the patient(s)).

Implementations of Framework 6 include TherapyView, which is a demonstration system to help therapists visualize the dynamic contents of past treatment sessions, enabled by neural topic modeling techniques to analyze the topical tendencies of various psychiatric conditions, and apply a deep learning-based image generation engine to provide a visual summary of the semantic content and topic representations of past and present treatment sessions. The system incorporates temporal modeling to provide a time-series representation of topic similarities at a turn-level resolution and AI-generated artworks applied to data derived at least from the dialogue segments to provide a concise representations of the content of a treatment session, offering interpretable insights for therapists to optimize their strategies and enhance the effectiveness of psychotherapy. Evaluation and testing of the implementations of Framework 6 included an empirical evaluation of existing neural topic modeling techniques with a focus on their application to the domain of psychotherapy, benchmarked on the Alexander Street Counseling and Psychotherapy Transcripts dataset. By leveraging temporal modeling of topic models, a visual representation of the topical tendencies of psychiatric conditions is provided, allowing therapists to easily identify patterns and make informed decisions about their psychotherapy strategies. To that end, the implementations of Framework 6 make use of AI-generated art generated from different temporal data sets for a therapy session to provide a concise visual summary of the session. A user-friendly interface of the implementations and interactive visualizations make it easy for therapists to understand and interpret the results, leading to improved treatment outcomes for patients. The data visualization system described herein offers a powerful tool for advancing the field of psychotherapy and providing therapists with the real-time information they need to make informed decisions (e.g., about the direction of the treatment, and any needed modifications or adjustments thereof).

The implementations of Framework 6 include a psychotherapy topic modeling system similar to the system 100 depicted in FIG. 1 (in relation to Framework 1). The system 100 leverages natural language processing (NLP) techniques and neural topic modeling to extract valuable insights from psychotherapy session transcripts. As noted during the discussion of Framework 1, during a psychotherapy session, the dialogue between the patient and therapist is transcribed into pairs of turns, which are then used as the input data for the machine learning topic modeling system. In some examples, full records of a patient (or a cohort of patients having the same condition) may be provided, and can either be used as is before feature extraction, or truncated into segments based on timestamps or topic turns.

Features from the transcript data are extracted using NLP techniques and are fitted into neural topic models to generate a list of weighted topic words. These topic words provide important insights into the patient's condition and are often highly interpretable, making them valuable in the context of psychotherapy. The system of FIG. 1 offers a range of downstream tasks and user scenarios. The extracted weighted topics can be used to assess the progress of the therapy, identify potential issues in the patient's mental state, or suggest adjustments to the therapist's treatment strategies. These features can be incorporated into an intelligent AI assistant to help remind the therapist of important information during the session. Additionally, certain taboo topics such as those related to suicidal conversations can be flagged for the therapist's attention.

In some embodiments, to further analyze the transcript data, a temporal topic modeling (TMM) was used to compute turn-resolution topic scores. An example TMM process (similar to the one used for Framework 1) is reproduced below:

Temporal Topic Modeling (TTM)

| | |
|---|---|
| 1: | Learned topics T as references |
| 2: | for i = 1, 2, ..., N do |
| 3: | Automatically transcribe dialogue turn pairs (S^{p}ᵢ, S^{t}ᵢ) |
| 4: | for Tⱼ ∈ topics T do |
| 5: | Topic score W^{pi}ⱼ = similarity(Emb(Tⱼ),Emb(S^{p}ᵢ )) |
| 6: | Topic score W^{ti}ⱼ = similarity(Emb(Tⱼ),Emb(S^{t}ᵢ)) |
| 7: | end for |
| 8: | end for |

Thus, for example, if there are ten (10) learned topics, the topic score will be a ten-dimensional vector, with each dimension corresponding to a likelihood of the turn being in that topic. To account for the directional property of each turn with respect to a given topic, the cosine similarity between the embedded topic vector and the embedded turn vector is computed, instead of directly inferring the probability (as in traditional topic assignment problems). The Embedded Topic Model (ETM), which is used for temporal modeling as discussed in greater detail below, also models each word with a categorical distribution whose natural parameter is the inner product between a word embedding and an embedding of its assigned topic. In some examples, Word2Vec is used as the word embedding for both the topics and the turns.

As was also discussed with reference to FIGS. 1 and 3 regarding the performance of the topic modeling approach, an implementation based on a system configuration similar to that of FIG. 1 was tested and evaluated. Specifically, transcript sessions were separated into three categories based on the psychiatric conditions of the patients (anxiety, depression, and schizophrenia), with the topic models being trained for over 100 epochs at a batch size of 16. As with standard preprocessing for topic modeling, a lower bound of the word count was kept to 3, and the ratio of the upper bound of the word count to keep in topic training to be 0.3. The models were evaluated using a series of validated measurements of topic coherence and diversity. Specifically, an asymmetrical confirmation measure between top word pairs (smoothed conditional probability) for topic coherence, and the ratio between the size of the vocabulary in the topic words and the total number of words in the topics for topic diversity were used. Table 320 of FIG. 3 summarizes the evaluation results for the four models across the different psychiatric conditions, based on validated measurements of topic coherence and diversity. It should be noted that the Embedded Topic Model (ETM) yields relatively high topic coherence and diversity across all three psychiatric conditions in the Alex Street datasets, making it a suitable choice for the deployed system.

To ensure that the learned topics can be mapped from one clinical condition to another, a universal topic model was computed on the text corpus of the entire Alex Street psychotherapy database. Using this universal topic model, a 10-dimensional topic score was computed for each turn, corresponding to the 10 topics. The higher the score, the more positively correlated the turn is with the topic. This time-series matrix allows probing the dynamics of the dialogues within the topic space (e.g., visualized as a 3D trajectory in the “Therapy View” demonstration system). To provide interpretable insights, it is important to parse out the concepts behind the learned topics. To better understand the topics, the highest-scoring turns in the transcripts that correspond to each topic was parsed out. For example, topic 0 was about figuring out self-discovery and reminiscence, while topic 1 was about play. Topic 2 was about anger, fear, and sadness, while topic 3 was about counts. Topic 4 was about tiredness and decision-making, while topic 5 was about sickness, self-injuries, and coping mechanisms. Topic 6 was about explicit ways to deal with stress, such as keeping busy and reaching out for help, while topic 7 was about numbers. Topic 8 was about continuation and perseverance, while topic 9 was mostly chitchat, interjections, and transcribed prosody.

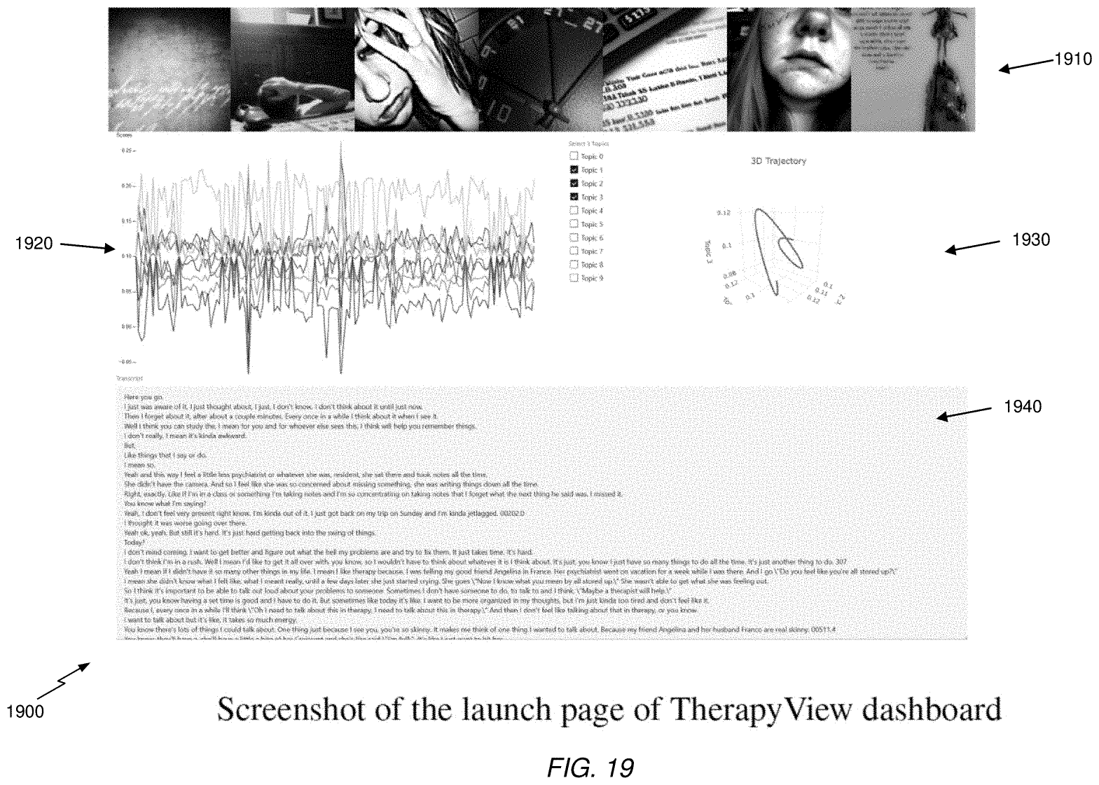

Next, the TherapyView demonstration implementation will be discussed. As noted, the outputs of the topic modeling framework (as depicted in FIG. 1), and the metrics and insights derived therefrom can be visualized on an example dashboard 1900 of the TherapyView platform shown in FIG. 19.

The TherapyView platform includes two parts: a Jupyter notebook that generates and serves the data, and a visual interactive dashboard (such as the dashboard 1900) that displays the data. There are four different visualizations in the dashboard:
Images (examples of which are shown in a first, top area 1910 of the dashboard 1900as a visual summary of this therapy session, powered by OpenAI's DALL-E 2 API.
Line graph of topical tendency over time (as the dialogue turns), examples of which are rendered shown in second area 1920 of the dashboard 1900.
3D plot, rendered in a third area 1930 of the dashboard 1900, showing the relationship of the selected three out of the ten topics over time.
A readout of the transcript (which can be replaced with user-specified inputs) rendered in a fourth area 1940 of the dashboard 1900.

In some examples, each AI-generated image in the top area 1910 of the dashboard represents a single chunk of 1,000 characters excerpt from the loaded transcript. In some embodiments, the input to the image-generating visualization tool can include a combination of the resultant topic modeling outputs generated by the topic modeling system (e.g., the system 100 of FIG. 1) with some selected content from the transcript (e.g., a machine learning selection model can be used to select one or more key transcript portions from each segment for which an image is to be generated). Other combinations of data to input to the AI-image generating tool may also be used.

The generated images act as a visual timeline, potentially surfacing notable changes in the patient during a session. The vague nature of these images is supplemented by the numerical data provided by the neural topic model. The therapist can explore each of the topics in detail through the charts described above. If the therapist finds a topic score change of interest, he/she can retrieve the corresponding line in the transcript and analyze the raw text.

This dashboard allows therapists to identify elements of concern by presenting them visually. By quickly identifying these elements, a therapist can provide the appropriate treatment in a timely fashion. These visualizations may also help identify surface behaviors that might have remained unnoticed by the therapist without the help of the dashboard.

The system architecture of the dashboard 1900 includes two main components: an API and a web application. The API is a single Jupyter notebook written in Python. This notebook contains all the logic for generating the visualizations in the dashboard. For example, the “Jupyter Kernel Gateway” package turns each cell into an API endpoint. The web component is a React single page application the queries the API for the data, displays it, and adds interactivity. It is noted that commercialization of the dashboard 1900 will likely require that the Jupyter notebook be replaced with a more robust solution.

Out of all the visualizations on the dashboard, the generated images are of special interest. Every refresh of the dashboard generates a new set of images, making the results unpredictable. Novel AI approaches, like DALL-E, even if they are imprecise, have the potential to provide new perspectives for a therapist to consider. Integrating DALL-E with real-world therapy does have some challenges:
(1) the API only allows a maximum of 1,000 characters per image request. This means that DALL-E cannot use any context outside of small chunks, which may limit the kind of insights that it has the potential to visualize.
(2) in the demo, a number of prompts were rejected by DALL-E for “safety” reasons. OpenAI prevents certain topics to be visualized for ethical reasons. Psychotherapy sessions can involve many sensitive topics and harmful behaviors. Further development of the visualization approach will require safeguards to ensure privacy and ethical use.

Accordingly, the data visualization demonstration system presents a visual journey through the doctor-patient dialogues in therapy sessions via temporal topic modeling and image generation. The results of this demonstration show that the Embedded Topic Model yields high topic coherence and diversity, making it a strong candidate for use in this domain. The incorporation of temporal modeling and interactive modules on the web dashboard provide additional interpretability, allowing therapists to better understand the progression of psychiatric conditions over time. The use of AI-generated artworks further enhances the interpretability of the results, providing therapists with a visual representation of the core themes of a given therapy session. The results of this study and demonstration provide valuable insights into the session trajectories of patients and therapists and have the potential to improve the effectiveness of psychotherapy. Additional features for the platform implemented for Framework 6 may include, for example, using the learned topic scores to predict psychological or therapeutic states with other digital traces. Additionally, chatbots will be trained as reinforcement learning agents using these states, incorporating biological and cognitive priors, and studying their factorial relations with other inference anchors, such as working alliance and personality. The ultimate goal is to construct a complete AI knowledge management system for mental health, utilizing different NLP annotations in real-time, and drive AI-augmented therapy sessions. The proposed TherapyView system described herein represents a novel approach to psychotherapy, leveraging the latest advancements in deep learning and data visualization to help therapists provide better care for their patients. The use of NLP and AI-generated arts in the system enables therapists to quickly identify patterns in patient data and tailor their treatment strategies accordingly.

Thus, in some embodiments, a system for visual representation of psychotherapy data is provided. The system includes a user interface to provide input and present output relating to the psychotherapy data, one or more memory devices to maintain time-dependent data associated with the psychotherapy data, and a processor-based controller in communication with the user interface and the one or more memory devices. The processor-based controller is configured to obtain transcript data representative of spoken dialog in one or more psychotherapy sessions conducted between a patient and a therapist, extract speech segments from the transcript data related to one or more of the patient or the therapist, apply a trained machine learning topic model process to the extracted speech segments to determine a temporal series of topic labels representative of semantic psychotherapy content of the extracted speech segments, determine a temporal visual representation of one or more of, for example, the topic labels of the temporal series and/or the transcript data, and render the temporal visual representation on an output device of the user interface. The above system can be implemented, at least in part, on a computing system executing instructions, stored on a non-transitory computer-readable media, to perform the visualization operations of Framework 6 as described herein.

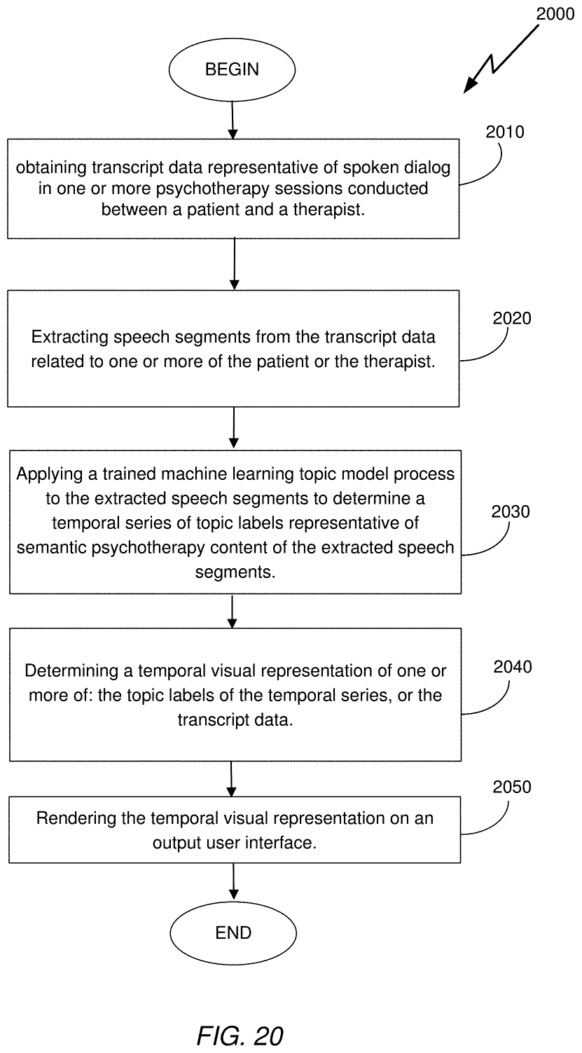

With reference next to FIG. 20, a flowchart of an example procedure 2000 for visual representation of psychotherapy data is shown. The procedure 2000 includes obtaining 2010 transcript data representative of spoken dialog in one or more psychotherapy sessions conducted between a patient and a therapist, extracting 2020 speech segments from the transcript data related to one or more of the patient or the therapist, and applying 2030 a trained machine learning topic model process to the extracted speech segments to determine a temporal series of topic labels representative of semantic psychotherapy content of the extracted speech segments. In some examples, applying the topic model process to the extracted speech segments may include transforming one or more of the extracted speech segments into representations in a vector space to produce one or more vectored speech segment representations, and determining one or more topic similarity scores between the one or more vectored speech segment representations and one or more vectored learned topics representations.

As further illustrated in FIG. 20, the procedure 2000 also includes determining 2040 a temporal visual representation of one or more of: the topic labels of the temporal series, or the transcript data, and rendering 2050 the temporal visual representation on an output user interface.

Determining the temporal visual representation may include determining for each temporal interval a representations of psychological state of the patient based on one or more of, for example, respective speech segments extracted from the transcript data, or respective portions of the temporal series of topic labels. In such embodiment, the rendering includes rendering at a first area of the output user interface an image generated by an AI-art-generating engine for the respective representation of the psychological state of the patient for the each temporal interval. In various examples, determining the temporal visual representation may include determining a time-dependent graph of tendency of at least some of the topic labels of the temporal series. In such examples, the rendering may include rendering the time-dependent graph in a second area of the output user interface.

In some examples, determining the temporal visual representation may include determining a time-dependent 3D plot showing the relationship over time of a selected subset of the topic labels of the temporal series. In such examples, the rendering may include rendering the time-dependent 3D plot in a third area of the output user interface. In further examples, determining the temporal visual representation may include dividing the transcript data into time-dependent portions. In such further examples, the rendering may include rendering at least some of the time-dependent portions of the transcript data in a fourth area of the output user interface.

### ADDITIONAL EMBODIMENTS

Performing the various techniques and operations described herein may be facilitated by a controller device (e.g., a processor-based computing device). Such a controller device may include a processor-based device such as a computing device, and so forth, that typically includes a central processor unit or a processing core. The device may also include one or more dedicated learning machines (e.g., neural networks) that may be part of the CPU or processing core. In addition to the CPU, the system includes main memory, cache memory and bus interface circuits. The controller device may include a mass storage element, such as a hard drive (solid state hard drive, or other types of hard drive), or flash drive associated with the computer system. The controller device may further include a keyboard, or keypad, or some other user input interface, and a monitor, e.g., an LCD (liquid crystal display) monitor, that may be placed where a user can access them.

The controller device is configured to facilitate, for example, processing and analyzing psychotherapy data (e.g., derived from psychotherapy transcript data). The storage device may thus include a computer program product that when executed on the controller device (which, as noted, may be a processor-based device) causes the processor-based device to perform operations to facilitate the implementation of procedures and operations described herein. The controller device may further include peripheral devices to enable input/output functionality. Such peripheral devices may include, for example, flash drive (e.g., a removable flash drive), or a network connection (e.g., implemented using a USB port and/or a wireless transceiver), for downloading related content to the connected system. Such peripheral devices may also be used for downloading software containing computer instructions to enable general operation of the respective system/device. Alternatively and/or additionally, in some embodiments, special purpose logic circuitry, e.g., an FPGA (field programmable gate array), an ASIC (application-specific integrated circuit), a DSP processor, a graphics processing unit (GPU), application processing unit (APU), etc., may be used in the implementations of the controller device. Other modules that may be included with the controller device may include a user interface to provide or receive input and output data. The controller device may include an operating system.

In implementations based on learning machines, different types of learning architectures, configurations, and/or implementation approaches may be used. Examples of learning machines include neural networks, including convolutional neural network (CNN), feed-forward neural networks, recurrent neural networks (RNN), etc. Feed-forward networks include one or more layers of nodes (“neurons” or “learning elements”) with connections to one or more portions of the input data. In a feedforward network, the connectivity of the inputs and layers of nodes is such that input data and intermediate data propagate in a forward direction towards the network's output. There are typically no feedback loops or cycles in the configuration/structure of the feed-forward network. Convolutional layers allow a network to efficiently learn features by applying the same learned transformation(s) to subsections of the data. Other examples of learning engine approaches/architectures that may be used include generating an auto-encoder and using a dense layer of the network to correlate with probability for a future event through a support vector machine, constructing a regression or classification neural network model that indicates a specific output from data (based on training reflective of correlation between similar records and the output that is to be identified), etc.

The neural networks (and other network configurations and implementations for realizing the various procedures and operations described herein) can be implemented on any computing platform, including computing platforms that include one or more microprocessors, microcontrollers, and/or digital signal processors that provide processing functionality, as well as other computation and control functionality. The computing platform can include one or more CPU's, one or more graphics processing units (GPU's, such as NVIDIA GPU's, which can be programmed according to, for example, a CUDA C platform), and may also include special purpose logic circuitry, e.g., an FPGA (field programmable gate array), an ASIC (application-specific integrated circuit), a DSP processor, an accelerated processing unit (APU), an application processor, customized dedicated circuity, etc., to implement, at least in part, the processes and functionality for the neural network, processes, and methods described herein. The computing platforms used to implement the neural networks typically also include memory for storing data and software instructions for executing programmed functionality within the device. Generally speaking, a computer accessible storage medium may include any non-transitory storage media accessible by a computer during use to provide instructions and/or data to the computer. For example, a computer accessible storage medium may include storage media such as magnetic or optical disks and semiconductor (solid-state) memories, DRAM, SRAM, etc.

The various learning processes implemented through use of the neural networks described herein may be configured or programmed using TensorFlow (an open-source software library used for machine learning applications such as neural networks). Other programming platforms that can be employed include keras (an open-source neural network library) building blocks, NumPy (an open-source programming library useful for realizing modules to process arrays) building blocks, etc.

Computer programs (also known as programs, software, software applications or code) include machine instructions for a programmable processor, and may be implemented in a high-level procedural and/or object-oriented programming language, and/or in assembly/machine language. As used herein, the term “machine-readable medium” refers to any non-transitory computer program product, apparatus and/or device (e.g., magnetic discs, optical disks, memory, Programmable Logic Devices (PLDs)) used to provide machine instructions and/or data to a programmable processor, including a non-transitory machine-readable medium that receives machine instructions as a machine-readable signal.

In some embodiments, any suitable computer readable media can be used for storing instructions for performing the processes/operations/procedures described herein. For example, in some embodiments computer readable media can be transitory or non-transitory. For example, non-transitory computer readable media can include media such as magnetic media (such as hard disks, floppy disks, etc.), optical media (such as compact discs, digital video discs, Blu-ray discs, etc.), semiconductor media (such as flash memory, electrically programmable read only memory (EPROM), electrically erasable programmable read only Memory (EEPROM), etc.), any suitable media that is not fleeting or not devoid of any semblance of permanence during transmission, and/or any suitable tangible media. As another example, transitory computer readable media can include signals on networks, in wires, conductors, optical fibers, circuits, any suitable media that is fleeting and devoid of any semblance of permanence during transmission, and/or any suitable intangible media.

Although particular embodiments have been disclosed herein in detail, this has been done by way of example for purposes of illustration only, and is not intended to be limiting with respect to the scope of the appended claims, which follow. Features of the disclosed embodiments can be combined, rearranged, etc., within the scope of the invention to produce more embodiments. Some other aspects, advantages, and modifications are considered to be within the scope of the claims provided below. The claims presented are representative of at least some of the embodiments and features disclosed herein. Other unclaimed embodiments and features are also contemplated.

What is claimed is:

1. A method for analyzing psychotherapy data, the method comprising:
obtaining transcript data representative of spoken dialog in one or more psychotherapy sessions conducted between a patient and a therapist;
extracting speech segments from the transcript data related to one or more of the patient or the therapist;
applying a trained machine learning topic model process to the extracted speech segments to determine weighted topic labels representative of semantic psychiatric content of the extracted speech segments; and
processing the weighted topic labels to derive a psychiatric assessment for the patient.

2. The method of claim 1, wherein the derived psychiatric assessment for the patient comprises one or more of: mental state of the patient, a therapy adjustment recommendation, or a trajectory of therapy for the patient.

3. The method of claim 1, wherein processing the weighted topic labels comprises applying a machine learning model to the weighted topic labels.

4. The method of claim 1, wherein applying the topic model process to the extracted speech segments comprises applying one or more of: a Latent Dirichlet Allocation (LDA) process, a Non Negative Matrix Factorization (NMF) process, a Latent Semantic Analysis (LSA) process, a Pachinko Allocation Model (PAM) process Neural Variational Document Model (NVDM) process, Wasserstein Latent Dirichlet Allocation (W-LDA) process, Embedded Topic Models (ETM) process, or a Bidirectional Adversarial Topic model (BATM) process.

5. The method of claim 1, wherein applying the topic model process to the extracted speech segments comprises:
transforming one or more of the extracted speech segments into representations in a vector space to produce one or more vectored topic label representations; and
determining one or more topic similarity scores between the one or more vectored topic label representations and one or more vectored representations of learned psychotherapy topic models.

6. The method of claim 1, wherein extracting the speech segments from the transcript data related to one or more of the patient or the therapist comprises:
extracting sequential temporal segments from the transcript data according to one or more extraction models comprising: pairing of dialog exchanges between the patient and the therapist, isolated patient-only speech segments, and isolated therapist-only speech segments.

7. A method for analyzing dialogue data, the method comprising:
transforming one or more patient speech segments and one or more speech segments of at least another speaker, representative of spoken dialogue between a patient and the at least other speaker, into representations in a vector space to produce one or more vectored patient representations and one or more vectored speaker representations;
determining one or more patient similarity scores between the one or more vectored patient representations and one or more vectored representations of a set of semantic elements in one or more inventories of cognitive properties;
determining one or more speaker similarity scores between the one or more vectored speaker representations and one or more vectored representations of another set of semantic elements in the one or more inventories of cognitive properties; and
determining based on the one or more patient similarity scores and the one or more speaker similarity scores a psychiatric assessment for the patient.

8. The method of claim 7, further comprising:
deriving the one or more vectored representations of the set of semantic elements and the one or more vectored representations the of the other set of semantic elements by transforming, into the vector space, therapy alliance semantic statements defining a Working Alliance Inventory (WAI) dataset, with the therapy alliance semantic statements being representative of therapeutic alliance of patient-perspective characteristics and therapist-perspective characteristics of one or more psychotherapy sessions.

9. The method of claim 8, wherein the patient-perspective characteristics and the therapist-perspective characteristics represent one or more of: collaborative nature of the patient's and a therapist's relationship, an affective bond between the therapist and the patient, and capabilities of the patient and the therapist to agree on treatment-related short-term tasks and long-term goals.

10. The method of claim 7, further comprising:
deriving the one or more vectored representations of the set of semantic elements and the one or more vectored representations the of the other set of semantic elements by transforming, into the vector space, semantic content based on a Myers-Briggs type indicator (MBTI) inventory, with the semantic content based on the MBTI inventory being representative of personality traits and behavioral trajectories for the patient and the at least other speaker.

11. The method of claim 7, wherein the at least other speaker includes one or more of:
one or more family members of the patient, one or more friends of the patients, one or more therapists, or one or more other patients participating in one or more group therapy sessions.

12. The method of claim 7, wherein the psychiatric assessment for the patient comprises one or more of: mental state of the patient, a therapy adjustment recommendation, or a trajectory of therapy for the patient.

13. The method of claim 7, wherein transforming the one or more patient speech segments and the one or more speech segments of the at least other speaker comprises:
transforming the speech segments using a neural network that includes a word embedding layer.

14. The method of claim 7, further comprising:
obtaining transcript data representative of the spoken dialogue in one or more events involving the patient and the at least other speaker; and
extracting from the transcript data the one or more data patient speech segments and the one or more speaker speech segments.

15. The method of claim 14, wherein obtaining transcript data comprises:
receiving multi-speaker audio data; and
performing speech separation for the multi-speaker audio data to identify respective speech utterances for the patient and the at least other speaker.

16. The method of claim 7, further comprising:
deriving a feature vector based at least on the one or more patient similarity scores and the one or more speaker similarity scores; and
providing the feature vector to a machine-learning sequence classifier to determine a psychological state for the patient.

17. A method for processing psychotherapy session data, the method comprising:
obtaining a current speech segment, representative of spoken dialogue between a patient and a therapist during a dialogue session comprising multiple speech segments;
transforming the current speech segment into a representation in a vector space to produce one or more vectored patient representations and one or more vectored therapist representations;
determining one or more patient similarity scores between the one or more vectored patient representations and one or more vectored representations of a set of semantic elements in one or more inventories of cognitive properties;
determining one or more therapist similarity scores between the one or more vectored therapist representations and one or more vectored representations of another set of semantic elements in the one or more inventories of cognitive properties; and
determining based on the one or more patient similarity scores and/or the one or more therapist similarity scores therapist advice output to dynamically manage the dialogue session in real-time by identifying, in response to the current speech segment, therapy-relevant actionable items.

18. The method of claim 17, further comprising:
deriving the one or more vectored representations of the set of semantic elements and the one or more vectored representations the of the other set of semantic elements by transforming, into the vector space, therapy alliance semantic statements defining a Working Alliance Inventory (WAI) dataset, with the therapy alliance semantic statements being representative of therapeutic alliance of patient-perspective characteristics and therapist-perspective characteristics of at least the current speech segment of the dialogue session.

19. The method of claim 17, wherein the therapy-relevant actionable items include one or more of: identifying additional topics to be discussed in subsequent speech segments of the dialogue session, identifying strategies for distracting the patient, identifying suggestions for putting the patient at ease, identifying strategies for re-directing the dialogue session, or identifying recommended mental exercises to be performed by the patient.

20. The method of claim 17, wherein determining the output to dynamically manage the dialogue session by identifying therapy-relevant actionable items comprises:
determining the actionable items based on a configurable machine learning recommendation engine; and
adjusting weights of the configurable machine learning recommendation engine based on quality evaluation of a subsequent action taken by the therapist in view of the actionable items determined by the machine learning recommendation engine.

21. The method of claim 20, wherein adjusting the weights of the configurable machine learning recommendation engine comprises adjusting the weights of the configurable machine learning recommendation according to one or more reinforcement learning approaches that include: a deep deterministic policy gradients (DDPG) approach, a twin delayed DDPG approach, or a batch constrained Q-learning approach.

22. The method of claim 20, further comprising:
training the recommendation engine according to one or more disorder-specific multi-objective policies using respective disorder-specific training datasets.

23. The method of claim 22, further comprising:
generating visualization outputs representing interpretable insights for the one or more disorder-specific multi-objective policies the recommendation engine was trained for, the visualization outputs comprising one or more of: topic trajectory plots for the one or more disorder-specific multi-objective policies, or transition matrices for the one or more disorder-specific multi-objective policies.

24. A method for multi-speaker diarization comprising:
obtaining a speech segment;
extracting one or more speech features from the speech segment;
processing the one or more extracted speech features with a configurable machine learning diarization engine adapted to identify a speaker associated with the speech segment; and
adjusting weights of the configurable machine learning diarization engine according to one or more reinforcement learning approaches in response to receipt of feedback indicative of accuracy of the speaker identified by the diarization engine to a true speaker identity for the speech segment.

25. The method of claim 24, wherein adjusting the weights of the configurable machine learning diarization engine comprises adjusting the weights of the configurable machine learning diarization engine according to one or more reinforcement learning approaches that include: a model-based reinforcement learning approach, a model-free reinforcement learning approach, an inverse reinforcement learning approach, or an imitation learning and behavioral cloning approach.

26. The method of claim 25, wherein any of the one or more reinforcement learning approaches is implemented according to one or more of: a deep learning process, a transfer learning process, a semi-supervised learning process, or a self-supervised learning as auxiliary model components process.

27. The method of claim 24, wherein adjusting the weights of the configurable machine learning diarization engine further comprises:
determining that the speaker associated with the speech segment is a new speaker not previously associated with previous speech segments processed by the machine learning diarization engine; and
configuring the machine learning diarization engine to generate a new label, associated with the new speaker, in response to processing subsequently obtained speech segments associated with the new speaker.

28. The method of claim 24, wherein adjusting the weights of the configurable machine learning diarization engine further comprises one or more of:
performing deep-learning-based reinforcement learning to adjust the weights of the configurable machine learning diarization engine;
performing batched and offline reinforcement learning to adjust the weights of the configurable machine learning diarization engine; or performing transfer learning process to adjust the weights of the configurable machine learning diarization engine based on existing weights of one or more other trained configurable machine learning diarization engines.

29. A knowledge management system comprising:
a user interface to provide input and present output relating to one or more documents;
one or more memory devices to maintain a relational database storing information relating to the one or more documents; and
a processor-based controller, in communication with the user interface and the one or more memory devices, to, for a particular document:
determine at a first time instance metadata information elements associated with the particular document;
include in a particular record of the relational database associated with the particular document at least some of the metadata information elements determined at the first time instance in one or more of a plurality of fields of the particular record, wherein the plurality of fields includes at least: a) a document-specific concepts field to maintain concepts specific to the particular document, and b) common concepts field to maintain common concepts shared by a plurality of documents associated with a plurality of records in the relational database; and
include in the particular record of the relational database, at one or more subsequent time instances, one or more document-specific user notes for storage in a document-specific notes field, and one or more general document user notes, determined by a machine learning engine analyzing other records in the relational database, for storage in a common notes field of multiple records of the relational database sharing the general user notes.

30. The knowledge management system of claim 29, wherein the particular document includes one of: a scholarly article written by a user, or user records for the user.

31. The knowledge management system of claim 29, wherein the processor-based controller configured to determine the metadata information elements is configured to:
divide the particular document into one or more semantic segments; and
apply one or more machine learning processes to the one or more semantic segments to derive annotation data for the particular document.

32. The knowledge management system of claim 31, wherein the processor-based controller configured to apply the one or more machine learning processes to derive annotation data for the particular document is configured to:
perform topic modeling analysis on one or more of: the one or more semantic segments of the particular document, or segments of other documents associated with other records of the relational database.

33. The knowledge management system of claim 31, wherein the processor-based controller configured to apply the one or more machine learning processes to derive annotation data for the particular document is configured to:
determine, using a vector-transformation-based machine learning engine, semantic similarity between the one or more segments of the particular document and one or more semantic items in at least one inventory of topics and concepts.

34. The knowledge management system of claim 31, wherein the processor-based controller configured to apply the one or more machine learning processes to derive annotation data for the particular document is configured to:
generate semantic summarization for the particular document based on one or more of: an extractive summarization techniques, or latent semantic analysis technique.

35. The knowledge management system of claim 31, wherein the processor-based controller configured to apply the one or more machine learning processes to derive annotation data for the particular document is configured to:
perform a symbolic reasoning analysis on the one or more segments of the particular document to determine logical and causal relationship between concepts associated with the semantic content of the one or more segments for the particular document.

36. The knowledge management system of claim 29, wherein the processor-based controller is further configured to:
determine, using a machine learning process, at least one of the common concepts shared by the plurality of documents based on semantic similarity between the concepts specific to the particular document and respective document-specific concepts for at least some of the plurality of documents.

37. The knowledge management system of claim 29, wherein the one or more documents comprises transcripts generated for psychotherapy sessions.

38. A method for visual representation of psychotherapy data, the method comprising:
obtaining transcript data representative of spoken dialog in one or more psychotherapy sessions conducted between a patient and a therapist;
extracting speech segments from the transcript data related to one or more of the patient or the therapist;
applying a trained machine learning topic model process to the extracted speech segments to determine a temporal series of topic labels representative of semantic psychotherapy content of the extracted speech segments;
determining a temporal visual representation of one or more of: the topic labels of the temporal series, or the transcript data; and
rendering the temporal visual representation on an output user interface.

39. The method of claim 38, wherein determining the temporal visual representation comprises:
determining for each temporal interval a representations of psychological state of the patient based on one or more of: respective speech segments extracted from the transcript data, or respective portions of the temporal series of topic labels; and
rendering at a first area of the output user interface an image generated by an AI-art-generating engine for the respective representation of the psychological state of the patient for each temporal interval.

40. The method of claim 38, wherein determining the temporal visual representation comprises:
determining a time-dependent graph of tendency of at least some of the topic labels of the temporal series; and
rendering the time-dependent graph in a second area of the output user interface.

41. The method of claim 38, wherein determining the temporal visual representation comprises:
determining a time-dependent 3D plot showing relationship over time of a selected subset of the topic labels of the temporal series; and
rendering the time-dependent 3D plot in a third area of the output user interface.

42. The method of claim 38, wherein determining the temporal visual representation comprises:
dividing the transcript data into time-dependent portions; and
rendering at least some of the time-dependent portions of the transcript data in a fourth area of the output user interface.

43. The method of claim 38, wherein applying the topic model process to the extracted speech segments comprises:
transforming one or more of the extracted speech segments into representations in a vector space to produce one or more vectored speech segment representations; and
determining one or more topic similarity scores between the one or more vectored speech segment representations and one or more vectored learned topics representations.
